SEQUENCE LISTING (6 sequences)

SEQ ID NO: 1 - DNA, 20 bp, Artificial Sequence
  Oligonucleotide Primer
cacccaagaacagggtttgt

SEQ ID NO: 2 - DNA, 21 bp, Artificial Sequence
  Oligonucleotide Primer
tggccatgggtatgttgttaa

SEQ ID NO: 3 - DNA, 19 bp, Artificial Sequence
  Oligonucleotide Primer
ttaccgggctctgccatct

SEQ ID NO: 4 - DNA, 17 bp, Artificial Sequence
  Oligonucleotide Primer
tgcccgccatcatccta

SEQ ID NO: 5 - DNA, 23 bp, Artificial Sequence
  Oligonucleotide Primer
cgtctgttatgtaaaggatgcgt

SEQ ID NO: 6 - DNA, 21 bp, Artificial Sequence
  Oligonucleotide Primer
tcctcatcgccctcccatccc

(19) United States
(12) Patent Application Publication
(10) Pub. No.: US 2014/0128339 A1
(43) Pub. Date: May 8, 2014

(54) 2'-METHYL SUBSTITUTED NUCLEOSIDE DERIVATIVES AND METHODS OF USE THEREOF FOR THE TREATMENT OF VIRAL DISEASES

(71) Applicants: Vinay Girijavallabhan, Whippany, NJ (US); Stephane Bogen, Somerset, NJ (US); Quang T. Truong, Morganville, NJ (US); Ping Chen, Edison, NJ (US); Angela Kerekes, Plainfield, NJ (US); Frank Bennett, Cranford, NJ (US); Sara Esposite, Neptune, NJ (US); Qingmei Hong, Scotch Plains, NJ (US); Ian Davies, Princeton, NJ (US)

(72) Inventors: Vinay Girijavallabhan, Whippany, NJ (US); Stephane Bogen, Somerset, NJ (US); Quang T. Truong, Morganville, NJ (US); Ping Chen, Edison, NJ (US); Angela Kerekes, Plainfield, NJ (US); Frank Bennett, Cranford, NJ (US); Sara Esposite, Neptune, NJ (US); Qingmei Hong, Scotch Plains, NJ (US); Ian Davies, Princeton, NJ (US)

(21) Appl. No.: 14/054,424

(22) Filed: Oct. 15, 2013

Related U.S. Application Data
(60) Provisional application No. 61/715,035, filed on Oct. 17, 2012.

(51) Int. Cl. C07H 19/06; A61K 45/06; A61K 31/7072; C07H 19/10; A61K 31/7068

(52) U.S. Cl. CPC C07H 19/06; C07H 19/10; A61K 31/7068; A61K 31/7072; A61K 45/06
USPC 514/51; 536/28.51; 536/28.5; 536/28.53; 536/26.8; 536/26.26; 514/49; 514/50

(57) ABSTRACT

The present invention relates to 2′-Methyl Substituted Nucleoside Derivatives of Formula (I):

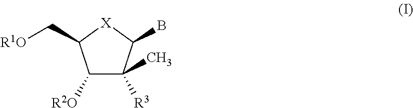

and pharmaceutically acceptable salts thereof, wherein R, R¹, R² and R³, are as defined herein. The present invention also relates to compositions comprising at least one 2′-Methyl Substituted Nucleoside Derivative, and methods of using the 2′-Methyl Substituted Nucleoside Derivatives for treating or preventing HCV infection in a patient.

### REFERENCE TO SEQUENCE LISTING SUBMITTED ELECTRONICALLY

The sequence listing of the present application is being submitted electronically via EFS-Web as an ASCII formatted sequence listing with a file name “23339USNP_SEQLIST_{—}15OCT2013.txt”, creation date of Oct. 15, 2013 and a size of 2 KB. This sequence listing submitted via EFS-Web is part of the specification and is herein incorporated by reference in its entirety.

### FIELD OF THE INVENTION

The present invention relates to 2′-Methyl Substituted Nucleoside Derivatives, compositions comprising at least one 2′-Methyl Substituted Nucleoside Derivative, and methods of using the 2′-Methyl Substituted Nucleoside Derivatives for treating or preventing HCV infection in a patient.

### BACKGROUND OF THE INVENTION

Hepatitis C virus (HCV) infection is a major health problem that leads to chronic liver disease, such as cirrhosis and hepatocellular carcinoma, in a substantial number of infected individuals, estimated to be 2-15% of the world's population. There are over 3 million chronically infected people in the United States alone, according to the U.S. Center for Disease Control. About 150 million individuals are chronically infected worldwide, with at least 3 to 4 million people being infected each year. Hepatitis C Fact Sheet, World Health Organization, July 2012. Once infected, about 20% of people clear the virus, but the rest harbor HCV the rest of their lives. Ten to twenty percent of chronically infected individuals eventually develop liver-destroying cirrhosis or cancer. HCV is transmitted parenterally by contaminated blood and blood products, contaminated needles, or sexually and vertically from infected mothers or carrier mothers to their off-spring.

Different approaches to HCV therapy have been taken, which include the inhibition of viral serine proteinase (NS3 protease), helicase, and RNA-dependent RNA polymerase (NS5B), and the development of a vaccine. Current and investigational treatments for HCV infection are reviewed in Poordad et al., Treating hepatitis C: current standard of care and emerging direct-acting antiviral agents. Journal of Viral Hepatitis 19: 449-464 (2012); Asselah et al., Protease and polymerase inhibitors for the treatment of hepatitis C. Liver International 29(s1): 57-67 (2009); G. J. Dore. The changing therapeutic landscape for hepatitis C. Med. J. Australia 196: 629-632 (2012); and Balsano, Mini Rev. Med. Chem. 8(4): 307-318, 2008. Despite the availability of therapeutic treatment options, chronic HCV infection remains a major healthcare concern. Moreover, there is no established vaccine for HCV. Consequently, there is a need for improved therapeutic agents that effectively combat chronic HCV infection.

The HCV virion is an enveloped positive-strand RNA virus with a single oligoribonucleotide genomic sequence of about 9400 bases which encodes a polyprotein of about 3,000 amino acids. The protein products of the HCV gene consist of the structural proteins C, E1, and E2, and the non-structural proteins NS2, NS3, NS4A, NS4B, NS5A and NS5B. The nonstructural (NS) proteins are believed to provide the catalytic machinery for viral replication.

The NS3 protease releases NS5B, the RNA-dependent RNA polymerase from the polyprotein chain. HCV NS5B polymerase is required for the synthesis of a negative-strand RNA intermediate from a positive-strand genomic viral RNA that serves as a template in the replication cycle of HCV. NS5B polymerase is an essential component in the HCV replication complex. See K. Ishi, et al., “Expression of Hepatitis C Virus NS5B Protein: Characterization of Its RNA Polymerase Activity and RNA Binding,” Hepatology, 29:1227-1235 (1999) and V. Lohmann, et al., “Biochemical and Kinetic Analyses of NS5B RNA-Dependent RNA Polymerase of the Hepatitis C Virus,” Virology, 249: 108-118 (1998). Inhibition of HCV NS5B polymerase prevents formation of the double-stranded HCV RNA and therefore constitutes an attractive approach to the development of HCV-specific antiviral therapies.

The development of inhibitors of HCV NS5B polymerase with potential for the treatment of HCV infection has been reviewed in Poordad et al. (2012), supra; Asselah et al. (2009), supra; and Chatel-Chaix et al. Direct-acting and host-targeting HCV inhibitors: current and future directions. Current Opinion in Virology, 2:588-598 (2012). The activity of purine ribonucleosides against HCV polymerase was reported by A. E. Eldrup et al., “Structure-Activity Relationship of Purine Ribonucleosides for Inhibition of HCV RNA-Dependent RNA Polymerase,” J. Med. Chem., 47:2283-2295 (2004). Nucleoside analogs said to be useful in the treatment of hepatitis C are disclosed in WO 2011/035231, WO 2005/003147, WO 2010/0081628, U.S. Pat. No. 7,879,815, WO 2010/075517, WO 2010/002877, and WO 2009/132123.

### SUMMARY OF THE INVENTION

In one aspect, the present invention provides Compounds of Formula (I):

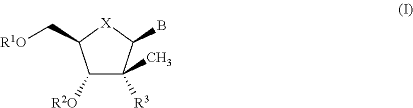

or a pharmaceutically acceptable salt thereof,
wherein:

B is:

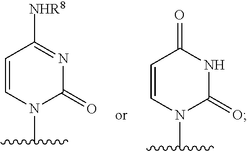

X is O, N, S or CH₂;

R¹ is H,

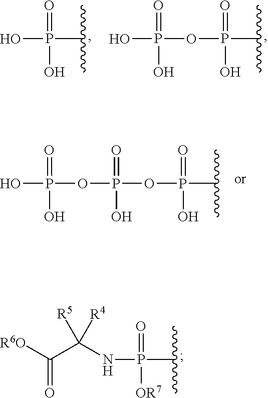

R² is H, —C(O)—(C₁-C₂₀ alkyl), —C(O)—(C₃-C₆ cycloalkyl), —C(O)O—(C₁-C₂₀ alkyl), —C(O)O—(C₃-C₆ cycloalkyl), —C(O)NH—(C₁-C₂₀ alkyl), —C(O)NH—(C₃-C₆ cycloalkyl) or

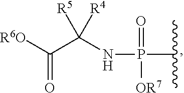

or R¹ and R² join to form a group having the formula:

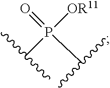

R³ is —CN, —N₃ or —C≡CH;

R⁴ is H, C₁-C₆ alkyl, C₃-C₇ cycloalkyl, phenyl or benzyl, wherein said C₁-C₆ alkyl can be optionally substituted with a group selected from halo, —OR¹², —SR¹², guanidino, —N(R¹²)₂—C(O)OR¹², —C(O)N(R¹²)₂, —NHC(O)R¹², 5- or 6-membered monocyclic heteroaryl and 9- or 10-membered bicyclic heteroaryl, and wherein said phenyl group and said benzyl group can be optionally substituted with up to 2 groups, each independently selected from C₁-C₆ alkyl, halo and —OR¹²;

R⁵ is H, C₁-C₆ alkyl, C₃-C₇ cycloalkyl, phenyl or benzyl, wherein said C₁-C₆ alkyl can be optionally substituted with a group selected from halo, —OR¹², —SR¹², guanidino, —N(R¹²)₂, —C(O)OR¹², —C(O)N(R¹²)₂, —NHC(O)R¹², 5- or 6-membered monocyclic heteroaryl and 9- or 10-membered bicyclic heteroaryl, and wherein said phenyl group and said benzyl group can be optionally substituted with up to 2 groups, each independently selected from C₁-C₆ alkyl, halo and —OR¹²;

R⁶ is H, C₁-C₂₀ alkyl, C₂-C₂₀ alkenyl, —(C₁-C₃ alkylene)ₘ-(C₃-C₇ cycloalkyl), —(C₁-C₃ alkylene)ₘ-(C₆-C₁₀ aryl) or —(C₁-C₃ alkylene)ₘ-adamantyl, wherein said C₁-C₂₀ alkyl group, said C₂-C₂₀ alkenyl group, said C₆-C₁₀ aryl group and said adamantyl group can be optionally substituted with up to three groups, each independently selected from halo, —OR¹², —C(O)OR¹², CN, NO₂, C₁-C₆ haloalkyl, C₁-C₆ hydroxyalkyl, C₃-C₇ cycloalkyl, C₆-C₁₀ aryl, 5- or 6-membered monocyclic heteroaryl, 9- or 10-membered bicyclic heteroaryl, —N(R¹²)₂, —C(O)N(R¹²)₂—SR¹², —S(O)R¹², —S(O)₂R¹², —S(O)₂N(R¹²)₂, —NHC(O)R¹², —NHC(O)OR¹² and —NHC(O)N(R¹²)₂;

R⁷ is H, C₆-C₁₀ aryl, 5- or 6-membered monocyclic heteroaryl, 9- or 10-membered bicyclic heteroaryl, or —(C₁-C₃ alkylene)-C(O)O—(C₁-C₆ alkyl), wherein said C₆-C₁₀ aryl group, said 5- or 6-membered monocyclic heteroaryl group and said 9- or 10-membered bicyclic heteroaryl group can be optionally substituted with R¹³;

R⁸ is H or —C(O)—(C₁-C₂₀ alkyl);

R¹¹ is H, C₁-C₂₀ alkyl, C₂-C₂₀ alkenyl, —(C₁-C₃ alkylene)ₘ-(C₃-C₇ cycloalkyl), C₃-C₇ cycloalkyl, —(C₁-C₃ alkylene)ₘ-C₆-C₁₀ aryl or —(C₁-C₃ alkylene)ₘ-adamantyl, 5- or 6-membered monocyclic heteroaryl, 9- or 10-membered bicyclic heteroaryl, wherein said C₁-C₂₀ alkyl group, said C₂-C₂₀ alkenyl group, said C₆-C₁₀ aryl group, said adamantyl group, said 5- or 6-membered monocyclic heteroaryl group and said 9- or 10-membered bicyclic heteroaryl group can be optionally substituted with up to five groups, each independently selected from C₁-C₆ alkyl, C₂-C₆ alkenyl, C₂-C₆ alkynyl, C₃-C₇ cycloalkyl, C₆-C₁₀ aryl, 5- or 6-membered monocyclic heteroaryl, 9- or 10-membered bicyclic heteroaryl, halo, —OR¹², —SR¹², —S(O)R¹², —S(O)₂R¹², —S(O)₂N(R¹²)₂, C₁-C₆ haloalkyl, C₁-C₆ hydroxyalkyl, —O—(C₁-C₆ haloalkyl), —CN, —NO₂, —N(R¹²)₂, —C(O)OR¹², —C(O)N(R¹²)₂ and —NHC(O)R¹², —NHC(O)OR¹² and —NHC(O)N(R¹²)₂;

each occurrence of R¹² is independently H, C₁-C₁₀ alkyl, C₁-C₆ haloalkyl, C₁-C₆ hydroxyalkyl, —(C₁-C₃ alkylene)ₘ-(C₃-C₇ cycloalkyl), —(C₁-C₃ alkylene)ₘ-(C₆-C₁₀ aryl), —(C₁-C₃ alkylene)ₘ-(4 to 7-membered heterocycloalkyl), —(C₁-C₃ alkylene)ₘ-(5- or 6-membered monocyclic heteroaryl) or —(C₁-C₃ alkylene)ₘ-(9- or 10-membered bicyclic heteroaryl), wherein said C₃-C₇ cycloalkyl group, said C₆-C₁₀ aryl group, said 4 to 7-membered heterocycloalkyl group, said-5- or 6-membered monocyclic heteroaryl group or said 9- or 10-membered bicyclic heteroaryl group can be optionally substituted with R¹⁶;

R¹³ represents from one to five substituent groups, each independently selected from C₁-C₆ alkyl, halo, —OR¹², —SR¹², C₁-C₆ haloalkyl, C₁-C₆ hydroxyalkyl, —O—(C₁-C₆ haloalkyl), —CN, —NO₂, —N(R¹²)₂, —C(O)OR¹², —C(O)N(R¹²)₂ and —NHC(O)R¹², or any two R¹³ groups on adjacent ring carbon atoms can combine to form —O—R¹⁴—O—;

R¹⁴ is —[C(R¹⁵)₂]ₙ—;

each occurrence of R¹⁵ is independently H or C₁-C₆ alkyl;

R¹⁶ represents from one to five substituent groups, each independently selected from C₁-C₆ alkyl, C₂-C₆ alkenyl, C₂-C₆ alkynyl, halo, —OR¹⁷, —SR¹⁷, C₁-C₆ haloalkyl, C₁-C₆ hydroxyalkyl, —O—(C₁-C₆ haloalkyl), —CN, —NO₂, —N(R¹⁷)₂, —C(O)OR¹⁷, —C(O)N(R¹⁷)₂ and —NHC(O)R¹⁷;

each occurrence of R¹⁷ is independently H, C₁-C₆ alkyl, C₁-C₆ haloalkyl, C₁-C₆ hydroxyalkyl, —(C₁-C₃ alkylene)ₘ-(C₃-C₇ cycloalkyl), —(C₁-C₃ alkylene)₆, —(C₆-C₁₀ aryl), —(C₁-C₃ alkylene)ₘ-(4 to 7-membered heterocycloalkyl), —(C₁-C₃ alkylene)ₘ-(5- or 6-membered monocyclic heteroaryl) or —(C₁-C₃ alkylene)ₘ-(9- or 10-membered bicyclic heteroaryl);

each occurrence of m is independently 0 or 1, and

each occurrence of n is independently 1, 2, or 3.

The Compounds of Formula (I) (also referred to herein as the “2′-Methyl Substituted Nucleoside Derivatives”) and pharmaceutically acceptable salts thereof can be useful, for example, for inhibiting HCV viral replication or replicon activity, for inhibiting HCV NS5B activity, and for treating or preventing HCV infection in a patient. Without being bound by any specific theory, it is believed that the 2′-Methyl Substituted Nucleoside Derivatives inhibit HCV viral replication by inhibiting HCV NS5B.

Accordingly, the present invention provides methods for treating or preventing HCV infection in a patient, comprising administering to the patient an effective amount of at least one 2′-Methyl Substituted Nucleoside Derivative.

The details of the invention are set forth in the accompanying detailed description set forth below.

Other embodiments, aspects and features of the present invention are either further described in or will be apparent from the ensuing description, examples and appended claims.

### DETAILED DESCRIPTION OF THE INVENTION

The present invention relates to 2′-Methyl Substituted Nucleoside Derivatives, compositions comprising at least one 2′-Methyl Substituted Nucleoside Derivative, and methods of using the 2′-Methyl Substituted Nucleoside Derivatives for treating or preventing HCV infection in a patient.

### DEFINITIONS AND ABBREVIATIONS

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc. . . .

As used herein, and throughout this disclosure, the following terms, unless otherwise indicated, shall be understood to have the following meanings:

A “patient” is a human or non-human mammal In one embodiment, a patient is a human. In another embodiment, a patient is a chimpanzee.

The term “effective amount” as used herein, refers to an amount of 2′-Methyl Substituted Nucleoside Derivative and/or an additional therapeutic agent, or a composition thereof that is effective in producing the desired therapeutic, ameliorative, inhibitory or preventative effect when administered to a patient suffering from a viral infection or virus-related disorder. In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to an HCV viral infection or HCV-virus related disorder, refers to reducing the likelihood or severity of HCV infection.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is linear. In another embodiment, an alkyl group is branched. Unless otherwise indicated, an alkyl group is unsubstituted.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkenyl” refers to an alkenyl group having from 2 to 6 carbon atoms. Unless otherwise indicated, an alkenyl group is unsubstituted.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkynyl” refers to an alkynyl group having from 2 to 6 carbon atoms. Unless otherwise indicated, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. In one embodiment, an aryl group can be optionally fused to a cycloalkyl or cycloalkanoyl group. Non-limiting examples of aryl groups include phenyl and naphthyl. In one embodiment, an aryl group is phenyl. Unless otherwise indicated, an aryl group is unsubstituted.

The term “arylene,” as used herein, refers to a bivalent group derived from an aryl group, as defined above, by removal of a hydrogen atom from a ring carbon of an aryl group. An arylene group can be derived from a monocyclic or multicyclic ring system comprising from about 6 to about 14 carbon atoms. In one embodiment, an arylene group contains from about 6 to about 10 carbon atoms. In another embodiment, an arylene group is a naphthylene group. In another embodiment, an arylene group is a phenylene group. An arylene group can be optionally substituted with one or more “ring system substituents” which may be the same or different, and are as defined herein below. An arylene group is divalent and either available bond on an arylene group can connect to either group flanking the arylene group. For example, the group “A-arylene-B,” wherein the arylene group is:

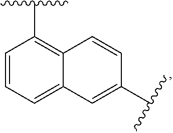 [SMILES: *c1ccc2c(*)cccc2c1]

is understood to represent both:

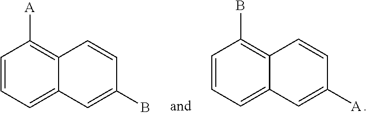

In one embodiment, an arylene group can be optionally fused to a cycloalkyl or cycloalkanoyl group. Non-limiting examples of arylene groups include phenylene and naphthalene. In one embodiment, an arylene group is unsubstituted. In another embodiment, an arylene group is:

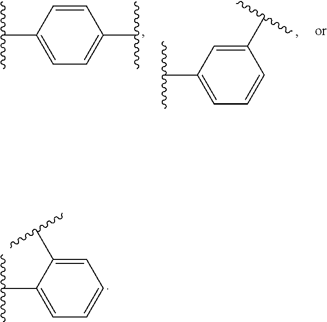 [SMILES: *c1ccc(*)cc1 ; *c1cccc(*)c1 ; *c1ccccc1*]

Unless otherwise indicated, an arylene group is unsubstituted.

The term “cycloalkyl,” as used herein, refers to a non-aromatic mono- or multicyclic ring system comprising from 3 to about 10 ring carbon atoms. In one embodiment, a cycloalkyl contains from about 5 to about 10 ring carbon atoms. In another embodiment, a cycloalkyl contains from 3 to about 7 ring atoms. In another embodiment, a cycloalkyl contains from about 5 to about 6 ring atoms. The term “cycloalkyl” also encompasses a cycloalkyl group, as defined above, which is fused to an aryl (e.g., benzene) or heteroaryl ring. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted. The term “3 to 6-membered cycloalkyl” refers to a cycloalkyl group having from 3 to 6 ring carbon atoms. Unless otherwise indicated, a cycloalkyl group is unsubstituted. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

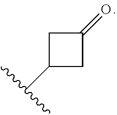 [SMILES: *C1CC(=O)C1]

The term “halo,” as used herein, means —F, —Cl, —Br or —I.

The term “haloalkyl,” as used herein, refers to an alkyl group as defined above, wherein one or more of the alkyl group's hydrogen atoms have been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CHF, —CHF₂, —CF₃, —CH₂Cl and —CCl₃. The term “C₁-C₆ haloalkyl” refers to a haloalkyl group having from 1 to 6 carbon atoms.

The term “hydroxyalkyl,” as used herein, refers to an alkyl group as defined above, wherein one or more of the alkyl group's hydrogen atoms have been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₆ hydroxyalkyl” refers to a hydroxyalkyl group having from 1 to 6 carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered heteroaryl. In another embodiment, a heteroaryl group comprises a 5- to 6-membered heteroaryl group fused to a benzene ring. Unless otherwise indicated, a heteroaryl group is unsubstituted.

The term “heterocycloalkyl,” as used herein, refers to a non-aromatic saturated monocyclic or multicyclic ring system comprising 3 to about 11 ring atoms, wherein from 1 to 4 of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic and has from 3 to about 7 ring atoms. In another embodiment, a heterocycloalkyl group is monocyclic has from about 4 to about 7 ring atoms. In another embodiment, a heterocycloalkyl group is bicyclic and has from about 7 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term “heterocycloalkyl” also encompasses a heterocycloalkyl group, as defined above, which is fused to an aryl (e.g., benzene) or heteroaryl ring. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone, silacyclopentane, silapyrrolidine and the like, and all isomers thereof. Non-limiting illustrative examples of a silyl-containing heterocycloalkyl group include:

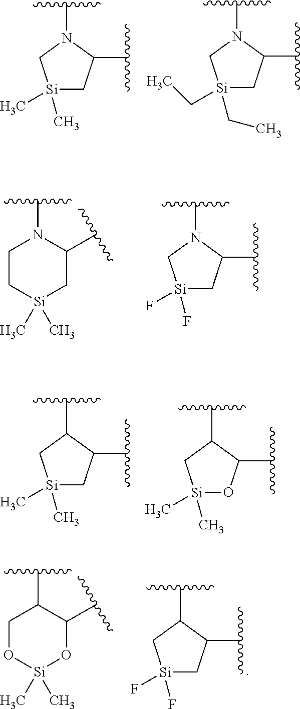

A ring carbon atom of a heterocycloalkyl group may be functionalized as a carbonyl group. An illustrative example of such a heterocycloalkyl group is:

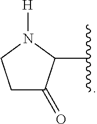 [SMILES: *C1NCCC1=O]

In one embodiment, a heterocycloalkyl group is a 5-membered monocyclic heterocycloalkyl. In another embodiment, a heterocycloalkyl group is a 6-membered monocyclic heterocycloalkyl. The term “3 to 6-membered monocyclic heterocycloalkyl” refers to a monocyclic heterocycloalkyl group having from 3 to 6 ring atoms. The term “4 to 6-membered monocyclic heterocycloalkyl” refers to a monocyclic heterocycloalkyl group having from 4 to 6 ring atoms. The term “7 to 11-membered bicyclic heterocycloalkyl” refers to a bicyclic heterocycloalkyl group having from 7 to 11 ring atoms. Unless otherwise indicated, an heterocycloalkyl group is unsubstituted.

The term “ring system substituent,” as used herein, refers to a substituent group attached to an aromatic or non-aromatic ring system which, for example, replaces an available hydrogen on the ring system. Ring system substituents may be the same or different, and are each independently selected. Examples of ring system substituents include alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, —Si(alkyl)₂, —Si(aryl)₂, —Si(heteroaryl)₂, —Si(alkyl)(aryl), —Si(alkyl)(cycloalkyl), —Si(alkyl)(heteroaryl), cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y₁)(Y₂), -alkylene-N(Y₁)(Y₂), —C(O)N(Y₁)(Y₂) and —S(O)₂N(Y₁)(Y₂), wherein Y₁ and Y₂ can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as for example:

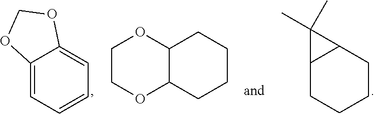

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any substituent or variable (e.g., alkyl, R⁶, R^{a}, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a 2′-Methyl Substituted Nucleoside Derivative or a pharmaceutically acceptable salt of the compound. The transformation may occur by various mechanisms (e.g., by metabolic or chemical processes), such as, for example, through hydrolysis in blood.

For example, if a 2′-Methyl Substituted Nucleoside Derivative or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as (-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a 2′-Methyl Substituted Nucleoside Derivative contains an alcohol functional group, a prodrug can be formed by the replacement of one or more of the hydrogen atoms of the alcohol groups with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, or glycosyl (the radical resulting from the removal of a hydroxyl group of the hemiacetal form of a carbohydrate). Other non-limiting example of alcohol-derived prodrugs include —P(O)(OH)₂; —P(O)(—O—C₁-C₆alkyl)₂; —P(O)(—NH-(α-aminoacyl group))(—O-aryl); —P(O)(—O—(C₁-C₆ alkylene)-S-acyl)(—NH-arylalkyl); any cyclic phosphate ester that forms a bridge between two ribose hydroxyl groups, such as:

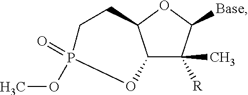

wherein the cyclic phosphate ester forms a bridge between the 3′-OH group and 5′-OH groups; and those described in U.S. Pat. No. 7,879,815; International Publication Nos. WO2005/003047, WO2008/082602, WO2010/0081628, WO2010/075517 and WO2010/075549; Mehellou, Chem. Med. Chem., 5:1841-1842 (2005); Bobeck et al., Antiviral Therapy 15:935-950 (2010); Furman et al., Future Medicinal Chemistry, 1:1429-1452 (2009); and Erion, Microsomes and Drug Oxidations, Proceedings of the International Symposium, 17th, Saratoga Springs, N.Y., United States, Jul. 6-10, 2008, 7-12 (2008).

If a 2′-Methyl Substituted Nucleoside Derivative incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇)cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl; carboxy(C₁-C₆)alkyl; amino(C₁-C₄)alkyl or mono-N— or di-N,N—(C₁-C₆)alkylaminoalkyl; —C(Y⁴)Y⁵ wherein Y⁴ is H or methyl and Y⁵ is mono-N— or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The 2′-Methyl Substituted Nucleoside Derivatives can form salts which are also within the scope of this invention. Reference to a 2′-Methyl Substituted Nucleoside Derivative herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a 2′-Methyl Substituted Nucleoside Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a 2′-Methyl Substituted Nucleoside Derivative with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the 2′-Methyl Substituted Nucleoside Derivatives may be atropisomers (e.g., substituted biaryls) and are considered as part of this invention. Enantiomers can also be directly separated using chiral chromatographic techniques.

It is also possible that the 2′-Methyl Substituted Nucleoside Derivatives may exist in different tautomeric forms, and all such forms are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a 2′-Methyl Substituted Nucleoside Derivative incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the 2′-Methyl Substituted Nucleoside Derivatives, and of the salts, solvates, hydrates, esters and prodrugs of the 2′-Methyl Substituted Nucleoside Derivatives, are intended to be included in the present invention.

In some instances, the compounds of the present invention are designated as “isomer 1” and “isomer 2.” This designation refers to stereoisomers at the chiral phosphorus atom of the 5′-prodrug moiety as illustrated below for cyclic and non-cyclic prodrugs, wherein the structure:

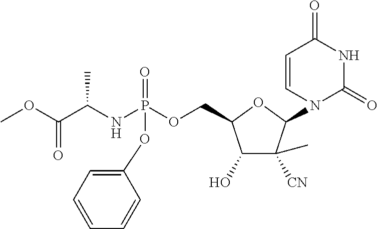 [SMILES: COC(=O)[C@H](C)NP(=O)(OC[C@H]1O[C@@H](n2ccc(=O)[nH]c2=O)[C@](C)(C#N)[C@@H]1O)Oc1ccccc1]

is understood to represent the following two phosphorus stereoisomers:

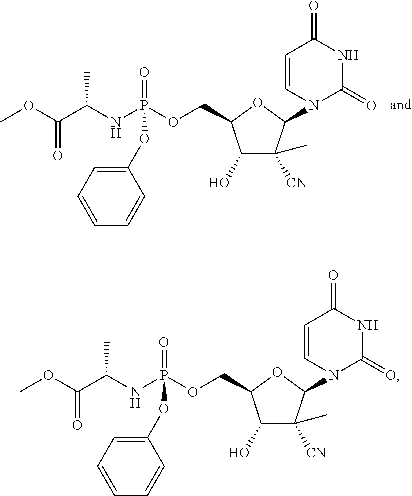 [SMILES: COC(=O)[C@H](C)N[P@@](=O)(OC[C@H]1O[C@@H](n2ccc(=O)[nH]c2=O)[C@](C)(C#N)[C@@H]1O)Oc1ccccc1 ; COC(=O)[C@H](C)N[P@](=O)(OC[C@H]1O[C@@H](n2ccc(=O)[nH]c2=O)[C@](C)(C#N)[C@@H]1O)Oc1ccccc1]

and the structure:

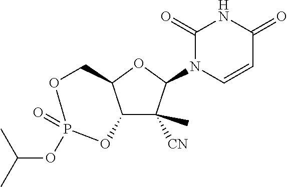

is understood to represent the following two phosphorus stereoisomers:

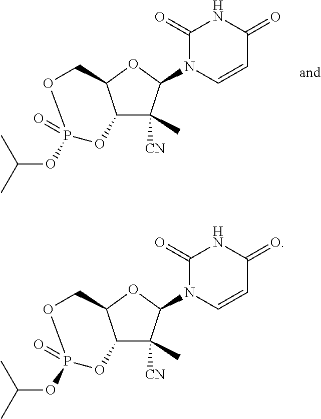 [SMILES: CC(C)O[P@]1(=O)OC[C@H]2O[C@@H](n3ccc(=O)[nH]c3=O)[C@](C)(C#N)[C@@H]2O1 ; CC(C)O[P@@]1(=O)OC[C@H]2O[C@@H](n3ccc(=O)[nH]c3=O)[C@](C)(C#N)[C@@H]2O1]

The terms “isomer 1” and “isomer 2” can be assigned to isomers of known absolute configuration or can be used to describe stereoisomers of unknown absolute configuration. Thus, the use of the terms “isomer 1” and “isomer 2” is not to be interpreted as indicating that the absolute configuration of both isomers is known.

The following abbreviations are used below and have the following meanings: Ac is acetyl or —C(O)CH₃, Ac₂O is acetic anhydride; Bu is butyl; t-BuMgCl is tert-butyl magnesium chloride; DCM is dichloromethane; Dess-Martin Periodinane is 1,1,1-triacetoxy-1,1-dihydro-1,2-benziodoxol-3(1H)-one; DIBAL-His diisobutylaluminum hydride; DMAP is N,N-dimethylamino pyridine; Et₃N (alternatively NEt₃) is triethylamine; EtOAc is ethyl acetate; EtOH is ethanol; HPLC is high performance liquid chromatography; KHMDS is potassium hexamethyldisilazide; KOBut is potassium tert-butoxide; LCMS is liquid chromatography/mass spectrometry; LDA is lithium diisopropylamide; MeOH is methanol; NMI is N-methylimidazole; Pd(OH)₂ is palladium hydroxide; TBAF is tetra n-butylammonium fluoride; TEA is triethylamine; THF is tetrahydrofuran; TIPDSiCl₂ is 1,3-dichloro-1,1,3,3-tetraisopropyldisiloxane; TLC is thin-layer chromatography; and TMSCN is trimethylsilyl cyanide.

### The Compounds of Formula (I)

The present invention provides 2′-Methyl Substituted Nucleoside Derivatives of Formula (I):

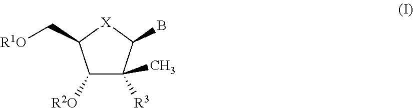

and pharmaceutically acceptable salts thereof, wherein B, R¹, R² and R³ are defined above for the Compounds of Formula (I).

In one embodiment, X is O.

In another embodiment, X is N.

In another embodiment, X is S.

In another embodiment, X is CH₂.

In one embodiment, R¹ is

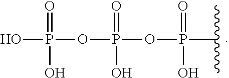 [SMILES: *P(=O)(O)OP(=O)(O)OP(=O)(O)O]

In another embodiment, R¹ is

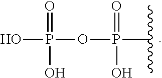 [SMILES: *P(=O)(O)OP(=O)(O)O]

In another embodiment, R¹ is

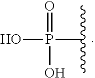 [SMILES: *P(=O)(O)O]

In one embodiment, R¹ and R² join to form a group having the formula:

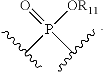

In another embodiment, R¹ and R² join to form a group having the structure:

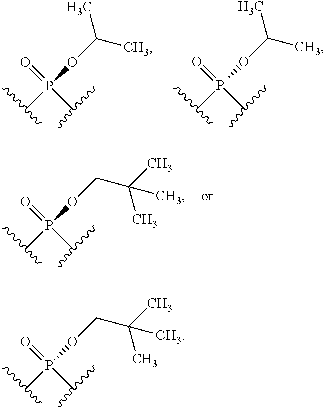

In one embodiment, R⁷ is phenyl, wherein said phenyl group can be optionally substituted with a halo group.

In one embodiment, the compounds of formula (I) have the formula (Ia):

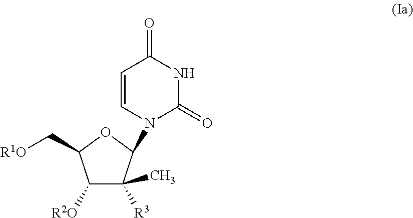

or a pharmaceutically acceptable salt thereof,
wherein:

R¹ is:

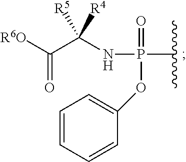

R² is H or —C(O)(C₁-C₆ alkyl);

R³ is —CN, —N₃ or —C≡CH;

R⁴ is H;

R⁵ is C₁-C₆ alkyl; and

R⁶ is C₁-C₆ alkyl.

In one embodiment, for the compounds of Formula (I) or Formula (Ia), B is:

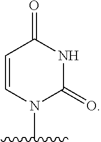 [SMILES: *n1ccc(=O)[nH]c1=O]

In another embodiment, for the compounds of Formula (I) or Formula (Ia), B is:

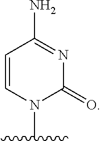 [SMILES: *n1ccc(N)nc1=O]

In one embodiment, for the compounds of Formula (I) or Formula (Ia), R² is H.

In another embodiment, for the compounds of Formula (I) or Formula (Ia), R² is —C(O)—(C₁-C₆ alkyl).

In another embodiment, for the compounds of Formula (I) or Formula (Ia), R² is —C(O)—CH(CH₃)₂.

In one embodiment, for the compounds of Formula (I) or Formula (Ia), R³ is —CN

In another embodiment, for the compounds of Formula (I) or Formula (Ia), R³ is —N₃.

In another embodiment, for the compounds of Formula (I) or Formula (Ia), R³ is —C≡CH.

In one embodiment, for the compounds of Formula (I) or Formula (Ia), R¹ is

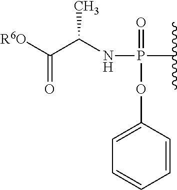

and R⁶ is C₁-C₆ alkyl.

In another embodiment, for the compounds of Formula (I) or Formula (Ia), R¹ is

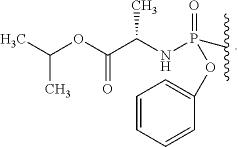 [SMILES: *P(=O)(N[C@@H](C)C(=O)OC(C)C)Oc1ccccc1]

In another embodiment, for the compounds of Formula (I) or Formula (Ia), R¹ is

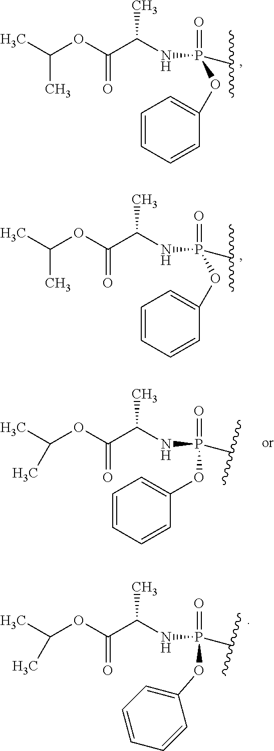

In one embodiment, variables B, R¹, R² and R³ for the Compounds of Formula (I) are selected independently of each other.

In another embodiment, the Compounds of Formula (I) are in substantially purified form.

The Compounds of Formula (I) may be referred to herein by chemical structure and/or by chemical name. In the instance that both the structure and the name of a Compound of Formula (I) are provided and a discrepancy is found to exist between the chemical structure and the corresponding chemical name, it is understood that the chemical structure will predominate.

Other embodiments of the present invention include the following:

(a) A pharmaceutical composition comprising an effective amount of a Compound of Formula (I) or a pharmaceutically acceptable salt thereof, and a pharmaceutically acceptable carrier.

(b) The pharmaceutical composition of (a), further comprising a second therapeutic agent selected from the group consisting of HCV antiviral agents, immunomodulators, and anti-infective agents.

(c) The pharmaceutical composition of (b), wherein the HCV antiviral agent is an antiviral selected from the group consisting of HCV protease inhibitors, HCV NS5B polymerase inhibitors and HCV NS5A inhibitors.

(d) A pharmaceutical combination that is (i) a Compound of Formula (I) and (ii) a second therapeutic agent selected from the group consisting of HCV antiviral agents, immunomodulators, and anti-infective agents; wherein the Compound of Formula (I) and the second therapeutic agent are each employed in an amount that renders the combination effective for inhibiting HCV replication, or for treating HCV infection and/or reducing the likelihood or severity of symptoms of HCV infection.

(e) The combination of (d), wherein the HCV antiviral agent is an antiviral selected from the group consisting of HCV protease inhibitors, HCV NS5B polymerase inhibitors and HCV NS5A inhibitors.

(f) A method of inhibiting HCV replication in a subject in need thereof which comprises administering to the subject an effective amount of a Compound of Formula (I).

(g) A method of treating HCV infection and/or reducing the likelihood or severity of symptoms of HCV infection in a subject in need thereof which comprises administering to the subject an effective amount of a Compound of Formula (I).

(h) The method of (g), wherein the Compound of Formula (I) is administered in combination with an effective amount of at least one second therapeutic agent selected from the group consisting of HCV antiviral agents, immunomodulators, and anti-infective agents.

(i) The method of (h), wherein the HCV antiviral agent is an antiviral selected from the group consisting of HCV protease inhibitors, HCV NS5B polymerase inhibitors and HCV NS5A inhibitors.

(j) A method of inhibiting HCV replication in a subject in need thereof which comprises administering to the subject the pharmaceutical composition of (a), (b) or (c) or the combination of (d) or (e).

(k) A method of treating HCV infection and/or reducing the likelihood or severity of symptoms of HCV infection in a subject in need thereof which comprises administering to the subject the pharmaceutical composition of (a), (b) or (c) or the combination of (d) or (e).

Additional embodiments of the invention include the pharmaceutical compositions, combinations and methods set forth in (a)-(k) above and the uses set forth in the discussion below, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate. It is understood that references to compounds would include the compound in its present form as well as in different forms, such as polymorphs, solvates and hydrates, as applicable.

It is further to be understood that the embodiments of compositions and methods provided as (a) through (k) above are understood to include all embodiments of the compounds, including such embodiments as result from combinations of embodiments.

Non-limiting examples of the Compounds of Formula (I) include compounds 1-38 as set forth below, and pharmaceutically acceptable salts thereof.

### Uses of the 2′-Methyl Substituted Nucleoside Derivatives

The 2′-Methyl Substituted Nucleoside Derivatives are useful in human and veterinary medicine for treating or preventing a viral infection in a patient. In one embodiment, the 2′-Methyl Substituted Nucleoside Derivatives can be inhibitors of viral replication. In another embodiment, the 2′-Methyl Substituted Nucleoside Derivatives can be inhibitors of HCV replication. Accordingly, the 2′-Methyl Substituted Nucleoside Derivatives are useful for treating viral infections, such as HCV.

The present invention also includes a compound of the present invention for use (i) in, (ii) as a medicament for, or (iii) in the preparation of a medicament for: (a) medicine, (b) inhibiting HCV replication or (c) treating HCV infection and/or reducing the likelihood or severity of symptoms of HCV infection. In these uses, the compounds of the present invention can optionally be employed in combination with one or more second therapeutic agents selected from HCV antiviral agents, anti-infective agents, and immunomodulators, as discussed in more detail, infra.

In one embodiment, the present invention includes the use of a compound of the present invention, or a pharmaceutically acceptable salt thereof, in a pharmaceutical composition for inhibiting HCV NS5B activity or for preventing and/or treating infection by HCV in a patient in need thereof.

In accordance with the invention, the 2′-Methyl Substituted Nucleoside Derivatives can be administered to a patient in need of treatment or prevention of a viral infection. Accordingly, in one embodiment, the invention provides methods for treating a viral infection in a patient comprising administering to the patient an effective amount of at least one 2′-Methyl Substituted Nucleoside Derivative or a pharmaceutically acceptable salt thereof.

### Treatment or Prevention of HCV Infection

The 2′-Methyl Substituted Nucleoside Derivatives are useful in the inhibition of HCV, the treatment of HCV infection and/or reduction of the likelihood or severity of symptoms of HCV infection and the inhibition of HCV viral replication and/or HCV viral production in a cell-based system. For example, the 2′-Methyl Substituted Nucleoside Derivatives are useful in treating infection by HCV after suspected past exposure to HCV by such means as blood transfusion, exchange of body fluids, bites, accidental needle stick, or exposure to patient blood during surgery or other medical procedures.

In one embodiment, the hepatitis C infection is acute hepatitis C. In another embodiment, the hepatitis C infection is chronic hepatitis C.

Accordingly, in one embodiment, the invention provides methods for treating HCV infection in a patient, the methods comprising administering to the patient an effective amount of at least one 2′-Methyl Substituted Nucleoside Derivative or a pharmaceutically acceptable salt thereof. In a specific embodiment, the amount administered is effective to treat or prevent infection by HCV in the patient. In another specific embodiment, the amount administered is effective to inhibit HCV viral replication and/or viral production in the patient.

The 2′-Methyl Substituted Nucleoside Derivatives are also useful in the preparation and execution of screening assays for antiviral compounds. For example the 2′-Methyl Substituted Nucleoside Derivatives are useful for identifying resistant HCV replicon cell lines harboring mutations within NS5B, which are excellent screening tools for more powerful antiviral compounds. Furthermore, the 2′-Methyl Substituted Nucleoside Derivatives are useful in establishing or determining the binding site of other antivirals to the HCV NS5B polymerase.

The compositions and combinations of the present invention can be useful for treating a patient suffering from infection related to any HCV genotype. HCV types and subtypes may differ in their antigenicity, level of viremia, severity of disease produced, and response to interferon therapy as described in Holland et al., Pathology, 30(2):192-195 (1998). The nomenclature set forth in Simmonds et al., J Gen Virol, 74(Pt11):2391-2399 (1993) is widely used and classifies isolates into six major genotypes, 1 through 6, with two or more related subtypes, e.g., 1a and 1b.

### Combination Therapy

In another embodiment, the present methods for treating or preventing HCV infection can further comprise the administration of one or more additional therapeutic agents which are not 2′-Methyl Substituted Nucleoside Derivatives.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a patient, the method comprising administering to the patient: (i) at least one 2′-Methyl Substituted Nucleoside Derivative (which may include two or more different 2′-Substituted Nucleoside Derivatives), or a pharmaceutically acceptable salt thereof, and (ii) at least one additional therapeutic agent that is other than a 2′-Methyl Substituted Nucleoside Derivative, wherein the amounts administered are together effective to treat or prevent a viral infection.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a 2′-Methyl Substituted Nucleoside Derivative and an additional therapeutic agent may be present in fixed amounts (dosage amounts) in a single dosage unit (e.g., a capsule, a tablet and the like).

In one embodiment, the at least one 2′-Methyl Substituted Nucleoside Derivative is administered during a time when the additional therapeutic agent(s) exert their prophylactic or therapeutic effect, or vice versa.

In another embodiment, the at least one 2′-Methyl Substituted Nucleoside Derivative and the additional therapeutic agent(s) are administered in doses commonly employed when such agents are used as monotherapy for treating a viral infection.

In another embodiment, the at least one 2′-Methyl Substituted Nucleoside Derivative and the additional therapeutic agent(s) are administered in doses lower than the doses commonly employed when such agents are used as monotherapy for treating a viral infection.

In still another embodiment, the at least one 2′-Methyl Substituted Nucleoside Derivative and the additional therapeutic agent(s) act synergistically and are administered in doses lower than the doses commonly employed when such agents are used as monotherapy for treating a viral infection.

In one embodiment, the at least one 2′-Methyl Substituted Nucleoside Derivative and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that can be treated or prevented using the combination therapy methods of the present invention include, but are not limited to, those listed above.

In one embodiment, the viral infection is HCV infection.

The at least one 2′-Methyl Substituted Nucleoside Derivative and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one 2′-Methyl Substituted Nucleoside Derivative and the additional therapeutic agent(s) may inhibit the resistance of a viral infection to these agents.

Non-limiting examples of additional therapeutic agents useful in the present compositions and methods include an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and any agent useful for treating an RNA-dependent polymerase-related disorder.

In one embodiment, one or more compounds of the invention are administered with one or more additional therapeutic agents, including but not limited to the therapeutic agents described, supra.

In one embodiment, the additional therapeutic agent is a viral protease inhibitor.

In another embodiment, the additional therapeutic agent is a viral replication inhibitor.

In another embodiment, the additional therapeutic agent is an HCV NS3 protease inhibitor.

In still another embodiment, the additional therapeutic agent is an HCV NS5B polymerase inhibitor.

In another embodiment, the additional therapeutic agent is a nucleoside inhibitor.

In another embodiment, the additional therapeutic agent is an interferon.

In yet another embodiment, the additional therapeutic agent is an HCV replicase inhibitor.

In another embodiment, the additional therapeutic agent is an antisense agent.

In another embodiment, the additional therapeutic agent is a therapeutic vaccine.

In a further embodiment, the additional therapeutic agent is a virion production inhibitor.

In another embodiment, the additional therapeutic agent is an antibody therapy.

In another embodiment, the additional therapeutic agent is an HCV NS2 inhibitor.

In still another embodiment, the additional therapeutic agent is an HCV NS4A inhibitor.

In another embodiment, the additional therapeutic agent is an HCV NS4B inhibitor.

In another embodiment, the additional therapeutic agent is an HCV NS5A inhibitor

In yet another embodiment, the additional therapeutic agent is an HCV NS3 helicase inhibitor.

In another embodiment, the additional therapeutic agent is an HCV IRES inhibitor.

In another embodiment, the additional therapeutic agent is an HCV p7 inhibitor.

In a further embodiment, the additional therapeutic agent is an HCV entry inhibitor.

In another embodiment, the additional therapeutic agent is an HCV assembly inhibitor.

In another embodiment, one or more compounds of the present invention are administered with one additional therapeutic agent selected from an HCV protease inhibitor, an interferon, a pegylated interferon and ribavirin. In another embodiment, one or more compounds of the present invention are administered with one additional therapeutic agent selected from an HCV polymerase inhibitor, a viral protease inhibitor, an interferon, and a viral replication inhibitor. In another embodiment, one or more compounds of the present invention are administered with ribavirin.

In still another embodiment, one or more compounds of the present invention are administered with two additional therapeutic agents selected from an HCV protease inhibitor, an HCV replication inhibitor, a nucleoside, an interferon, a pegylated interferon and ribavirin.

In another embodiment, one or more compounds of the present invention are administered with an HCV protease inhibitor and ribavirin. In another specific embodiment, one or more compounds of the present invention are administered with a pegylated interferon and ribavirin.

In another embodiment, one or more compounds of the present invention are administered with three additional therapeutic agents selected from an HCV protease inhibitor, an HCV replication inhibitor, a nucleoside, an interferon, a pegylated interferon and ribavirin.

In one embodiment, one or more compounds of the present invention are administered with two additional therapeutic agents selected from an HCV polymerase inhibitor, a viral protease inhibitor, an interferon, and a viral replication inhibitor.

In another embodiment, one or more compounds of the present invention are administered with ribavirin, interferon and another therapeutic agent.

In another embodiment, one or more compounds of the present invention are administered with ribavirin, interferon and another therapeutic agent, wherein the additional therapeutic agent is selected from an HCV polymerase inhibitor, a viral protease inhibitor, and a viral replication inhibitor.

In still another embodiment, one or more compounds of the present invention are administered with ribavirin, interferon and a viral protease inhibitor.

In another embodiment, one or more compounds of the present invention are administered with ribavirin, interferon and an HCV protease inhibitor.

In another embodiment, one or more compounds of the present invention are administered with ribavirin, interferon and boceprevir or telaprevir.

In a further embodiment, one or more compounds of the present invention are administered with ribavirin, interferon and an HCV polymerase inhibitor.

In another embodiment, one or more compounds of the present invention are administered with pegylated-interferon alpha and ribavirin.

In one embodiment, the additional therapeutic agents comprise a viral protease inhibitor and a viral polymerase inhibitor.

In still another embodiment, the additional therapeutic agents comprise a viral protease inhibitor and an immunomodulatory agent.

In yet another embodiment, the additional therapeutic agents comprise a polymerase inhibitor and an immunomodulatory agent.

In another embodiment, the additional therapeutic agents comprise a viral protease inhibitor and a nucleoside.

In another embodiment, the additional therapeutic agents comprise an immunomodulatory agent and a nucleoside.

In one embodiment, the additional therapeutic agents comprise an HCV protease inhibitor and an HCV polymerase inhibitor.

In another embodiment, the additional therapeutic agents comprise a nucleoside and an HCV NS5A inhibitor.

In another embodiment, the additional therapeutic agents comprise a viral protease inhibitor, an immunomodulatory agent and a nucleoside.

In a further embodiment, the additional therapeutic agents comprise a viral protease inhibitor, a viral polymerase inhibitor and an immunomodulatory agent.

In another embodiment, the additional therapeutic agent is ribavirin. HCV polymerase inhibitors useful in the present compositions and methods include, but are not limited to, VP-19744 (Wyeth/ViroPharma), PSI-7851 (Pharmasset), RG7128 (Roche/Pharmasset), PSI-7977 (Pharmasset), PSI-938 (Pharmasset), PSI-879 (Pharmasset), PSI-661 (Pharmasset), PF-868554/filibuvir (Pfizer), VCH-759NX-759 (ViroChem Pharma/Vertex), HCV-371 (Wyeth/VirroPharma), HCV-796 (Wyeth/ViroPharma), IDX-184 (Idenix), IDX-375 (Idenix), NM-283 (Idenix/Novartis), GL-60667 (Genelabs), JTK-109 (Japan Tobacco), PSI-6130 (Pharmasset), R1479 (Roche), R-1626 (Roche), R-7128 (Roche), MK-0608 (Isis/Merck), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK-3281 (Merck), VCH-222/VX-222 (ViroChem/Vertex), VCH-916 (ViroChem), VCH-716(ViroChem), GSK-71185 (Glaxo SmithKline), ANA598 (Anadys), GSK-625433 (Glaxo SmithKline), XTL-2125 (XTL Biopharmaceuticals), and those disclosed in Ni et al., Current Opinion in Drug Discovery and Development, 7(4):446 (2004); Tan et al., Nature Reviews, 1:867 (2002); and Beaulieu et al., Current Opinion in Investigational Drugs, 5:838 (2004).

Other HCV polymerase inhibitors useful in the present compositions and methods include, but are not limited to, those disclosed in International Publication Nos. WO 08/082,484, WO 08/082,488, WO 08/083,351, WO 08/136,815, WO 09/032,116, WO 09/032,123, WO 09/032,124 and WO 09/032,125; and the following compounds:

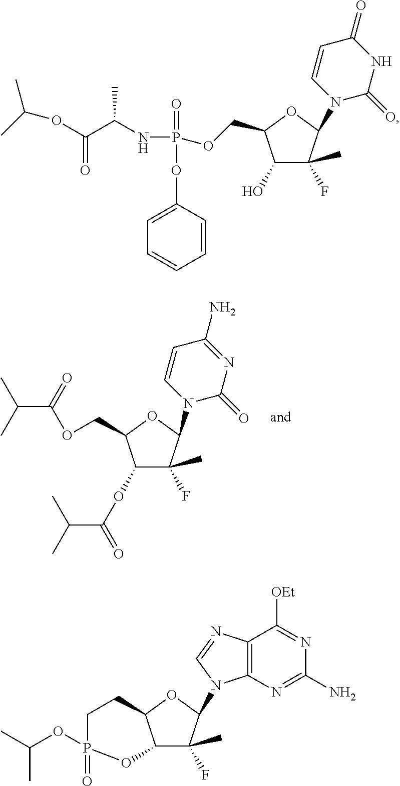

and pharmaceutically acceptable salts thereof.

Interferons useful in the present compositions and methods include, but are not limited to, interferon alfa-2a, interferon alfa-2b, interferon alfacon-1 and PEG-interferon alpha conjugates. “PEG-interferon alpha conjugates” are interferon alpha molecules covalently attached to a polyethylene glycol molecule. Illustrative PEG-interferon alpha conjugates include interferon alpha-2a (Roferon™, Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name PEG-interferon alpha-Intron™ from Schering-Plough Corporation), interferon alpha-2b-XL (e.g., as sold under the trade name PEG-Intron™), interferon alpha-2c (Berofor Alpha™, Boehringer Ingelheim, Ingelheim, Germany), PEG-interferon lambda (Bristol-Myers Squibb and ZymoGenetics), interferon alfa-2b alpha fusion polypeptides, interferon fused with the human blood protein albumin (Albuferon™, Human Genome Sciences), Omega Interferon (Intarcia), Locteron controlled release interferon (Biolex/OctoPlus), Biomed-510 (omega interferon), Peg-IL-29 (ZymoGenetics), Locteron CR (Octoplus), R-7025 (Roche), IFN-α-2b-XL (Flamel Technologies), belerofon (Nautilus) and consensus interferon as defined by determination of a consensus sequence of naturally occurring interferon alphas (Infergen™, Amgen, Thousand Oaks, Calif.).

Examples of viral protease inhibitors useful in the present compositions and methods include, but are not limited to, an HCV protease inhibitor. Examples of HCV protease inhibitors useful in the present compositions and methods include, but are not limited to, VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott/Enanta), TMC-435350 (Medivir), RG7227 (Danoprevir, InterMune/Roche), EA-058 (Abbott/Enanta), EA-063 (Abbott/Enanta), GS-9256 (Gilead), IDX-320 (Idenix), ACH-1625 (Achillion), ACH-2684 (Achillion), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), BMS-650032 (Bristol-Myers Squibb), VX-985 (Vertex) and PHX1766 (Phenomix).

Further examples of HCV protease inhibitors useful in the present compositions and methods include, but are not limited to, the following compounds:

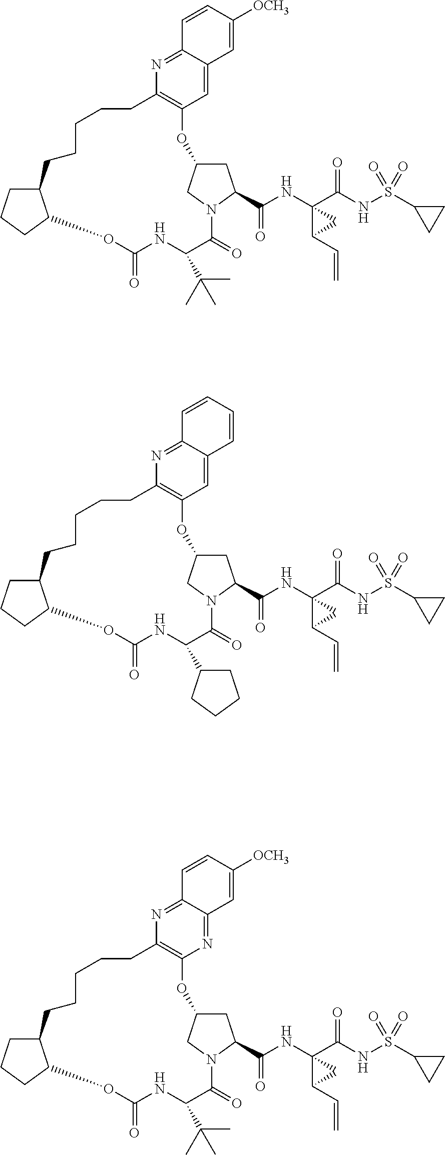

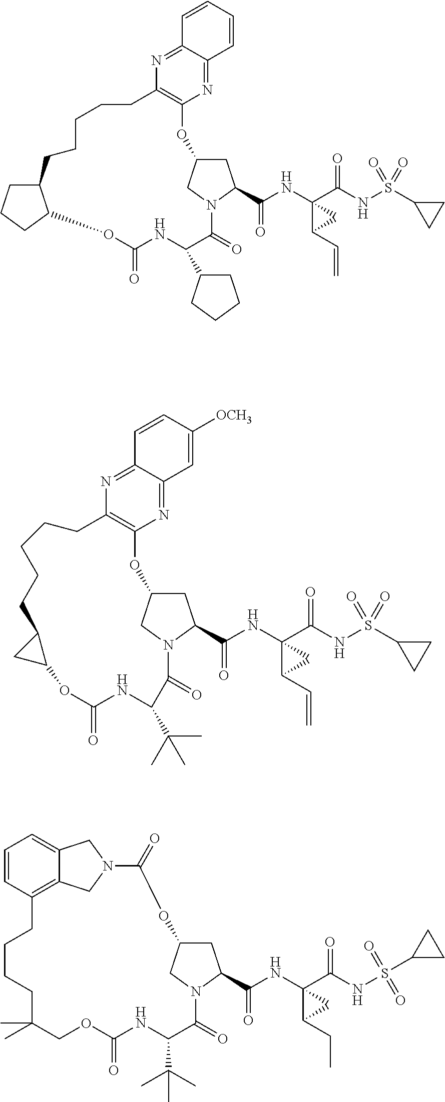

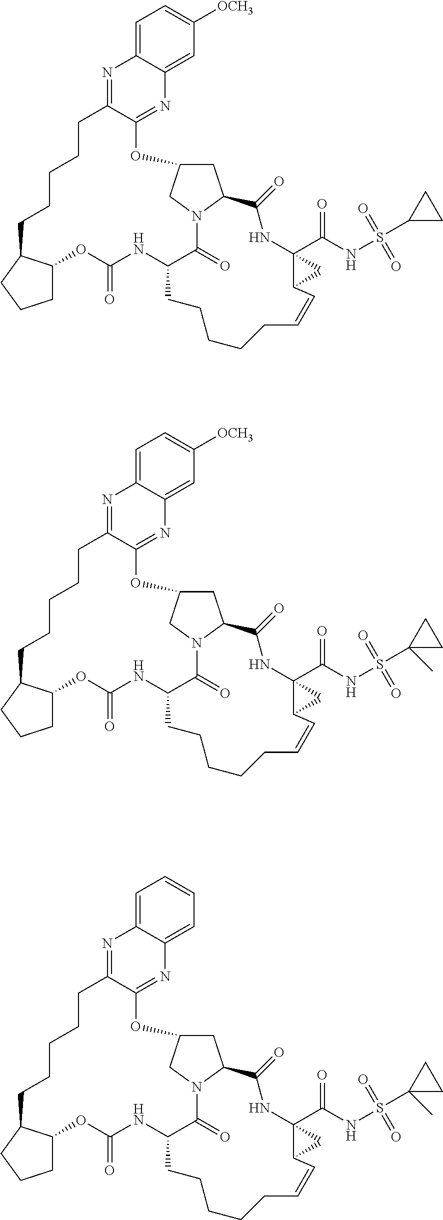 [SMILES: COc1ccc2nc3c(nc2c1)O[C@@H]1C[C@H]2C(=O)N[C@]4(C(=O)NS(=O)(=O)C5CC5)C[C@H]4/C=C\CCCCC[C@H](NC(=O)O[C@@H]4CCC[C@H]4CCCCC3)C(=O)N2C1 ; COc1ccc2nc3c(nc2c1)O[C@@H]1C[C@H]2C(=O)N[C@]4(C(=O)NS(=O)(=O)C5(C)CC5)C[C@H]4/C=C\CCCCC[C@H](NC(=O)O[C@@H]4CCC[C@H]4CCCCC3)C(=O)N2C1 ; CC1(S(=O)(=O)NC(=O)[C@@]23C[C@H]2/C=C\CCCCC[C@@H]2NC(=O)O[C@@H]4CCC[C@H]4CCCCCc4nc5ccccc5nc4O[C@@H]4C[C@@H](C(=O)N3)N(C4)C2=O)CC1]

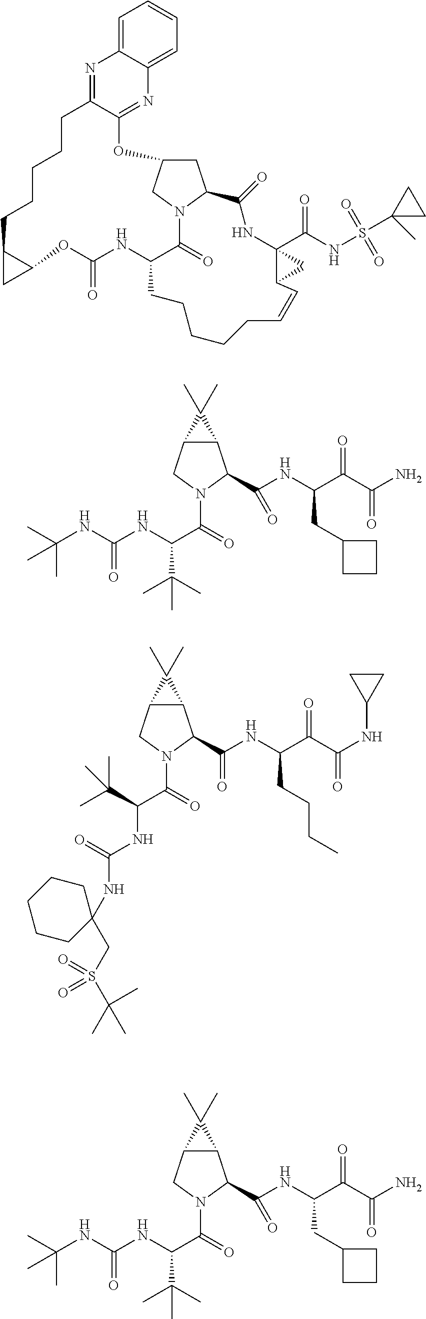

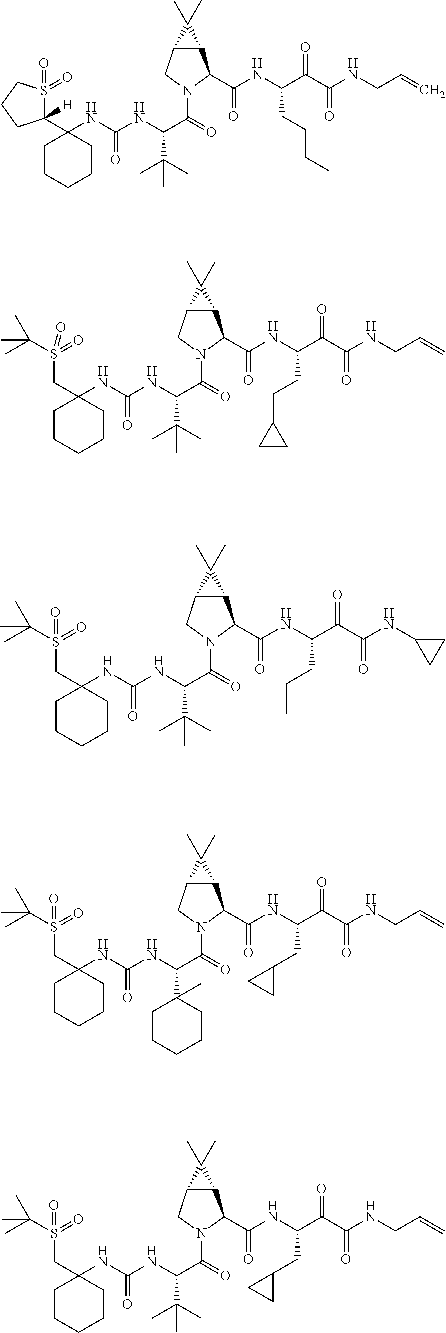

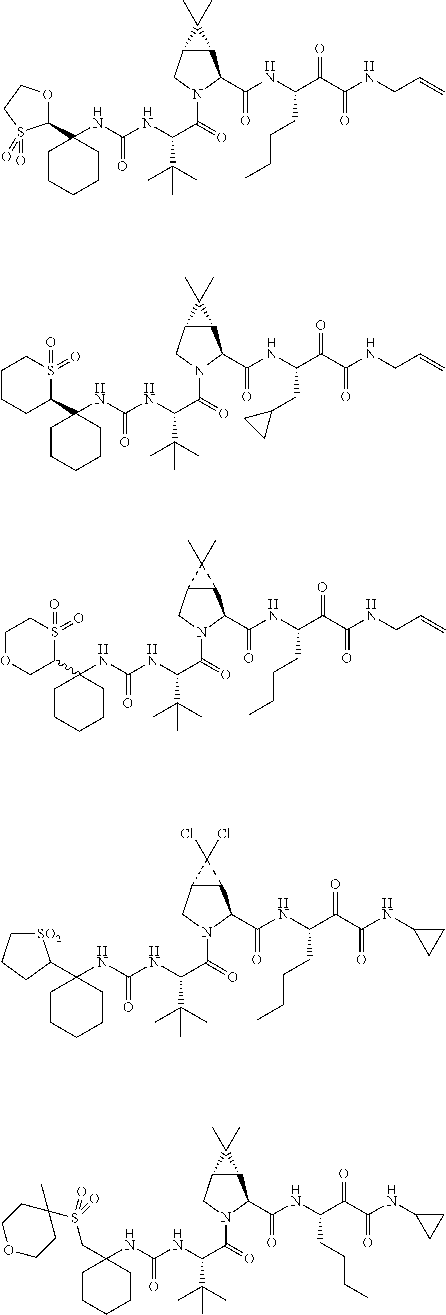

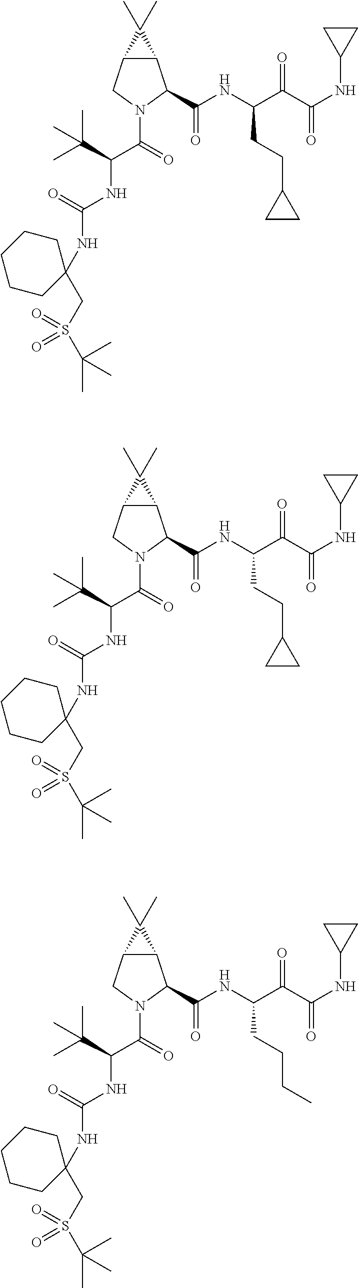 [SMILES: CC(C)(C)[C@H](NC(=O)NC1(CS(=O)(=O)C(C)(C)C)CCCCC1)C(=O)N1C[C@H]2[C@@H]([C@H]1C(=O)N[C@H](CCC1CC1)C(=O)C(=O)NC1CC1)C2(C)C ; CC(C)(C)[C@H](NC(=O)NC1(CS(=O)(=O)C(C)(C)C)CCCCC1)C(=O)N1C[C@H]2[C@@H]([C@H]1C(=O)N[C@@H](CCC1CC1)C(=O)C(=O)NC1CC1)C2(C)C ; CCCC[C@H](NC(=O)[C@@H]1[C@@H]2[C@H](CN1C(=O)[C@@H](NC(=O)NC1(CS(=O)(=O)C(C)(C)C)CCCCC1)C(C)(C)C)C2(C)C)C(=O)C(=O)NC1CC1]

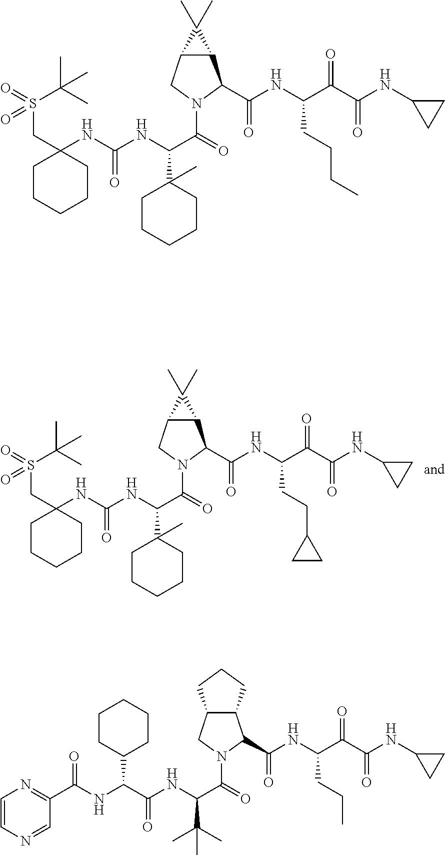

and pharmaceutically acceptable salts thereof.

Viral replication inhibitors useful in the present compositions and methods include, but are not limited to, HCV replicase inhibitors, IRES inhibitors, NS4A inhibitors, NS3 helicase inhibitors, NS5A inhibitors, NS5B inhibitors, ribavirin, AZD-2836 (Astra Zeneca), viramidine, A-831 (Arrow Therapeutics), EDP-239 (Enanta), ACH-2928 (Achillion), GS-5885 (Gilead); an antisense agent or a therapeutic vaccine.

HCV NS5A inhibitors useful in the present compositions and methods include, but are not limited to, ACH-2928 (Achilon), A-832 (Arrow Therpeutics), AZD-7295 (Astra Zeneca/Arrow), GS-5885 (Gilead), PPI-461 (Presidio), PPI-1301 (Presidio), BMS-824383 (Bristol-Myers Squibb) and BMS-790052 (Bristol-Myers Squibb). Additional HCV NS5A inhibitors useful as second additional therapeutic agents in the present compositions and methods include, but are not limited to those disclosed in International Publication No. WO 2010/111483 and the following compounds:

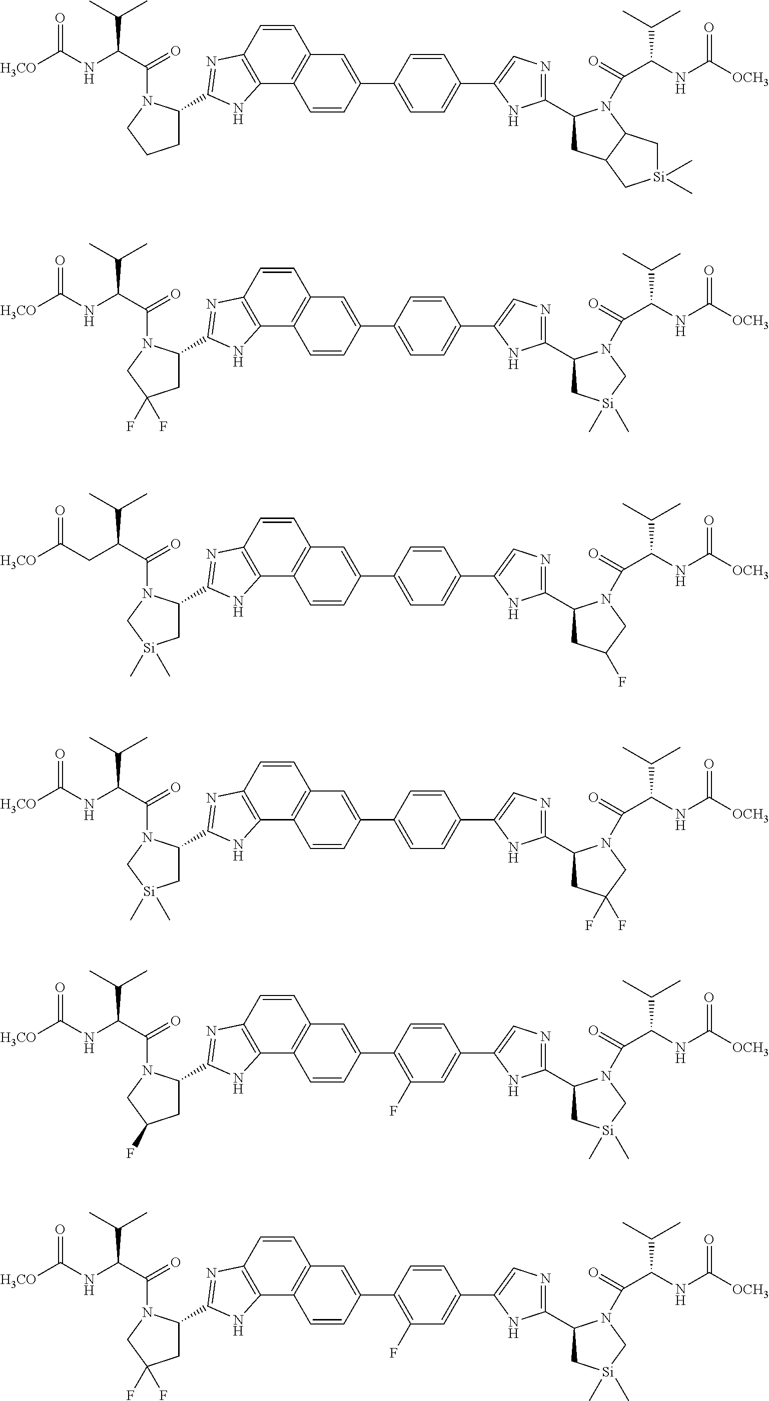

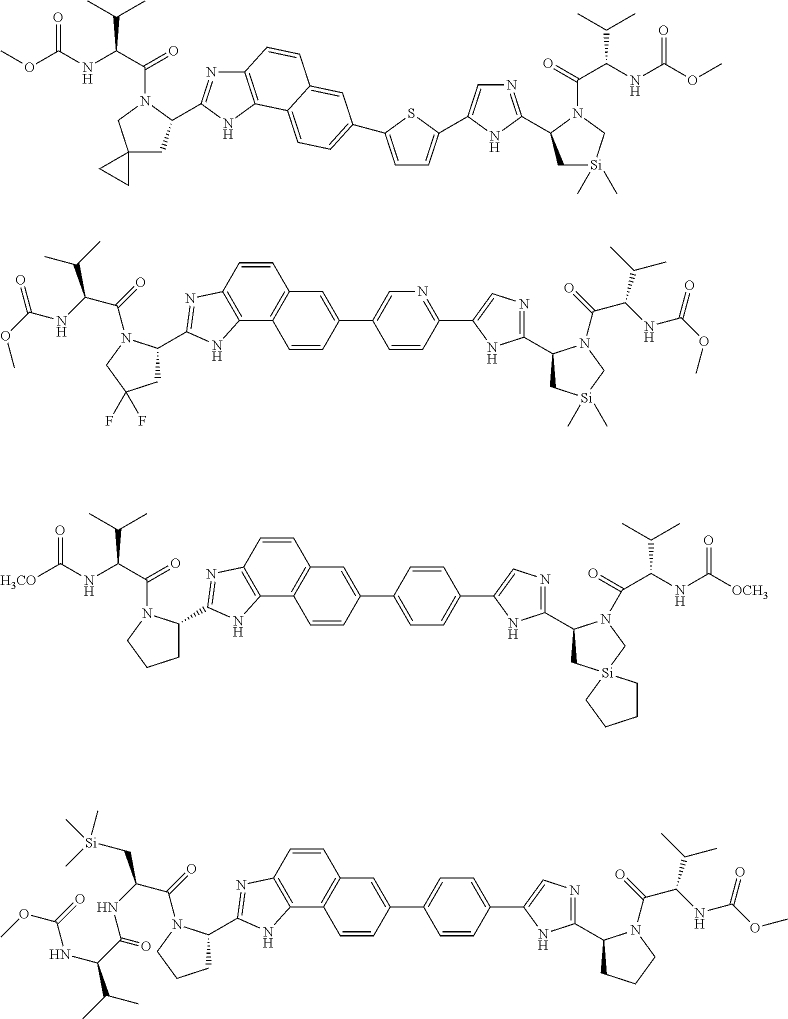

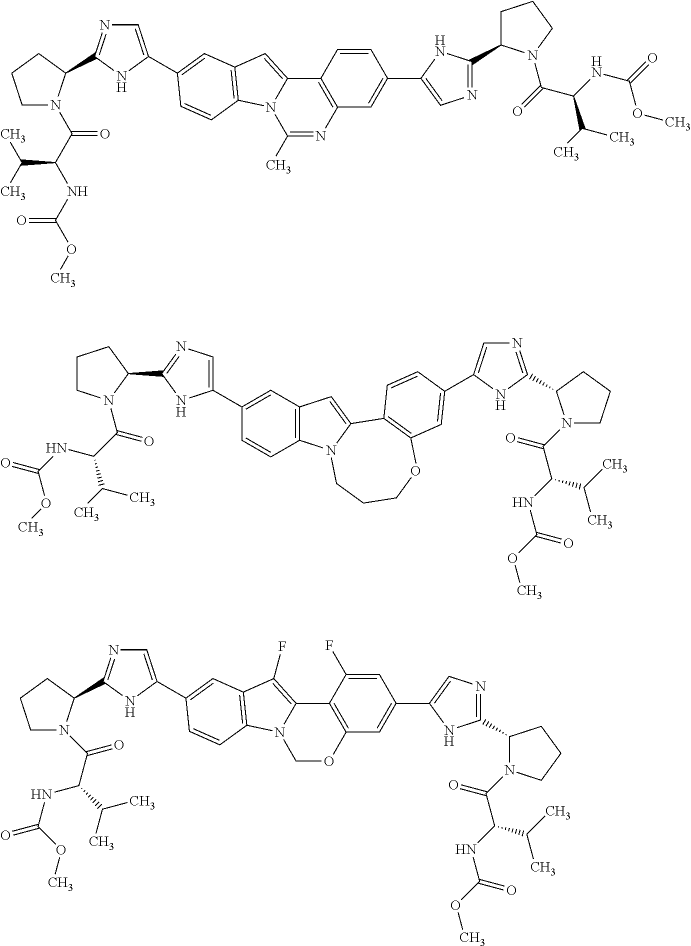 [SMILES: COC(=O)N[C@H](C(=O)N1CCC[C@@H]1c1ncc(-c2ccc3c(c2)nc(C)n2c4ccc(-c5cnc([C@@H]6CCCN6C(=O)[C@@H](NC(=O)OC)C(C)C)[nH]5)cc4cc32)[nH]1)C(C)C ; COC(=O)N[C@H](C(=O)N1CCC[C@H]1c1ncc(-c2ccc3c(c2)OCCCn2c-3cc3cc(-c4cnc([C@@H]5CCCN5C(=O)[C@@H](NC(=O)OC)C(C)C)[nH]4)ccc32)[nH]1)C(C)C ; COC(=O)N[C@H](C(=O)N1CCC[C@H]1c1ncc(-c2cc(F)c3c(c2)OCn2c-3c(F)c3cc(-c4cnc([C@@H]5CCCN5C(=O)[C@@H](NC(=O)OC)C(C)C)[nH]4)ccc32)[nH]1)C(C)C]

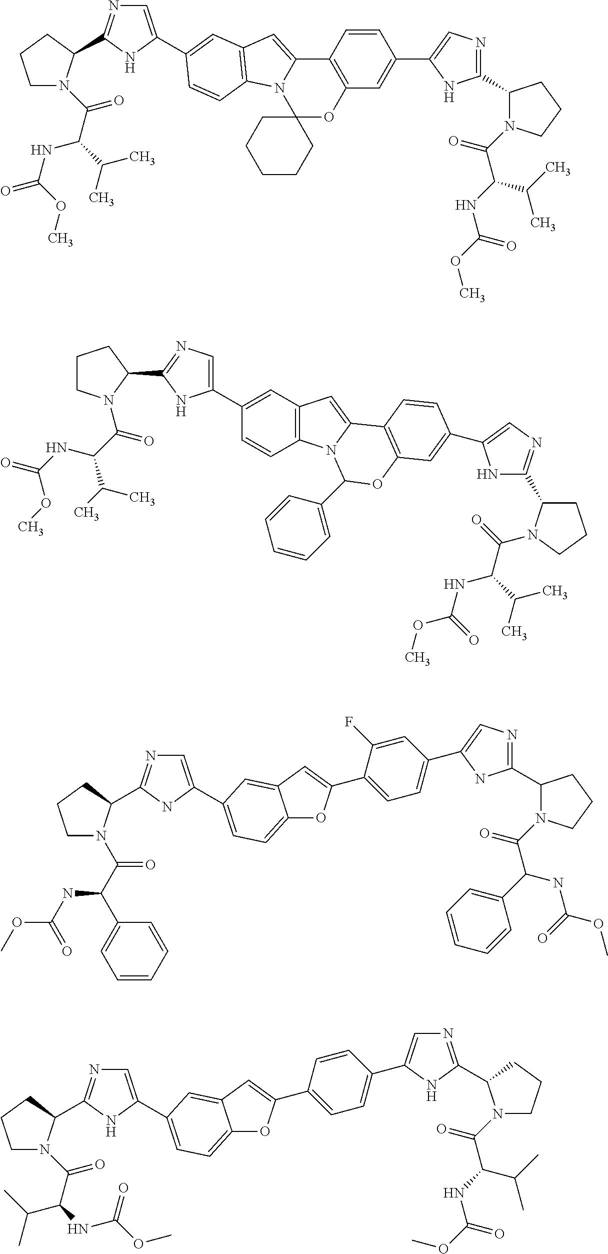

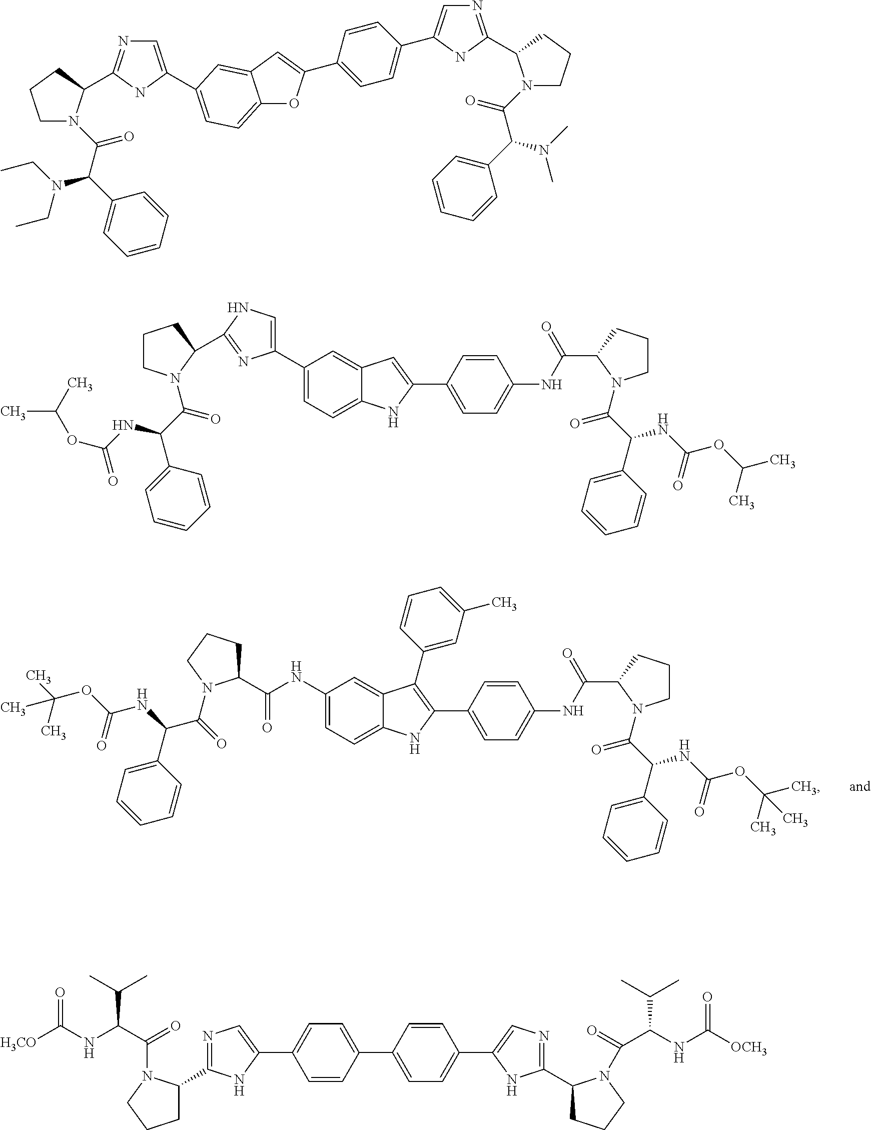

and pharmaceutically acceptable salts thereof.

HCV replicase inhibitors useful in the present compositions and methods include, but are not limited to, those disclosed in U.S. Patent Publication No. US20090081636.

The doses and dosage regimen of the other agents used in the combination therapies of the present invention for the treatment or prevention of HCV infection can be determined by the attending clinician, taking into consideration the approved doses and dosage regimen in the package insert; the age, sex and general health of the patient; and the type and severity of the viral infection or related disease or disorder. When administered in combination, the 2′-Methyl Substituted Nucleoside Derivative(s) and the other agent(s) can be administered simultaneously (i.e., in the same composition or in separate compositions one right after the other) or sequentially. This particularly useful when the components of the combination are given on different dosing schedules, e.g., one component is administered once daily and another component is administered every six hours, or when the preferred pharmaceutical compositions are different, e.g., one is a tablet and one is a capsule. A kit comprising the separate dosage forms is therefore advantageous.

Generally, a total daily dosage of the at least one 2′-Methyl Substituted Nucleoside Derivative(s) alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

In a further embodiment, when the additional therapeutic agent is Ribavirin (commercially available as REBETOL ribavirin from Schering-Plough or COPEGUS ribavirin from Hoffmann-La Roche), this agent is administered at a daily dosage of from about 600 to about 1400 mg/day for at least 24 weeks.

### Compositions and Administration

Due to their activity, the 2′-Methyl Substituted Nucleoside Derivatives are useful in veterinary and human medicine. As described above, the 2′-Methyl Substituted Nucleoside Derivatives are useful for treating or preventing HCV infection in a patient in need thereof.

When administered to a patient, the 2′-Methyl Substituted Nucleoside Derivatives can be administered as a component of a composition that comprises a pharmaceutically acceptable carrier or vehicle. The present invention provides pharmaceutical compositions comprising an effective amount of at least one 2′-Methyl Substituted Nucleoside Derivative and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more 2′-Methyl Substituted Nucleoside Derivatives are administered orally.

In another embodiment, the one or more 2′-Methyl Substituted Nucleoside Derivatives are administered intravenously.

In one embodiment, a pharmaceutical preparation comprising at least one 2′-Methyl Substituted Nucleoside Derivative is in unit dosage form. In such form, the preparation is subdivided into unit doses containing effective amounts of the active components.

Compositions can be prepared according to conventional mixing, granulating or coating methods, respectively, and the present compositions can contain, in one embodiment, from about 0.1% to about 99% of the 2′-Methyl Substituted Nucleoside Derivative(s) by weight or volume. In various embodiments, the present compositions can contain, in one embodiment, from about 1% to about 70% or from about 5% to about 60% of the 2′-Methyl Substituted Nucleoside Derivative(s) by weight or volume.

The quantity of 2′-Methyl Substituted Nucleoside Derivative in a unit dose of preparation may be varied or adjusted from about 1 mg to about 2500 mg. In various embodiment, the quantity is from about 10 mg to about 1000 mg, 1 mg to about 500 mg, 1 mg to about 100 mg, and 1 mg to about 100 mg.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The amount and frequency of administration of the 2′-Methyl Substituted Nucleoside Derivatives will be regulated according to the judgment of the attending clinician considering such factors as age, condition and size of the patient as well as severity of the symptoms being treated. Generally, a total daily dosage of the 2′-Methyl Substituted Nucleoside Derivatives range from about 0.1 to about 2000 mg per day, although variations will necessarily occur depending on the target of therapy, the patient and the route of administration. In one embodiment, the dosage is from about 1 to about 200 mg/day, administered in a single dose or in 2-4 divided doses. In another embodiment, the dosage is from about 10 to about 2000 mg/day, administered in a single dose or in 2-4 divided doses. In another embodiment, the dosage is from about 100 to about 2000 mg/day, administered in a single dose or in 2-4 divided doses. In still another embodiment, the dosage is from about 500 to about 2000 mg/day, administered in a single dose or in 2-4 divided doses.

The compositions of the invention can further comprise one or more additional therapeutic agents, selected from those listed above herein. Accordingly, in one embodiment, the present invention provides compositions comprising: (i) at least one 2′-Methyl Substituted Nucleoside Derivative or a pharmaceutically acceptable salt thereof; (ii) one or more additional therapeutic agents that are not a 2′-Methyl Substituted Nucleoside Derivative; and (iii) a pharmaceutically acceptable carrier, wherein the amounts in the composition are together effective to treat HCV infection.

In one embodiment, the present invention provides compositions comprising a Compound of Formula (I) and a pharmaceutically acceptable carrier.

In another embodiment, the present invention provides compositions comprising a Compound of Formula (I), a pharmaceutically acceptable carrier, and a second therapeutic agent selected from the group consisting of HCV antiviral agents, immunomodulators, and anti-infective agents.

In another embodiment, the present invention provides compositions comprising a Compound of Formula (I), a pharmaceutically acceptable carrier, and wto additional therapeutic agents, each of which are independently selected from the group consisting of HCV antiviral agents, immunomodulators, and anti-infective agents.

### Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below and generalized in Schemes A-M below. Alternative synthetic pathways and analogous structures will be apparent to those skilled in the art of organic synthesis.

Scheme A shows a method useful for making nucleoside compounds of formula A6, which correspond to the Compounds of Formula (I), wherein X is O; R¹ and R² are each H; R³ is methyl and B is:

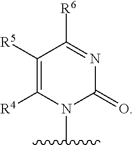

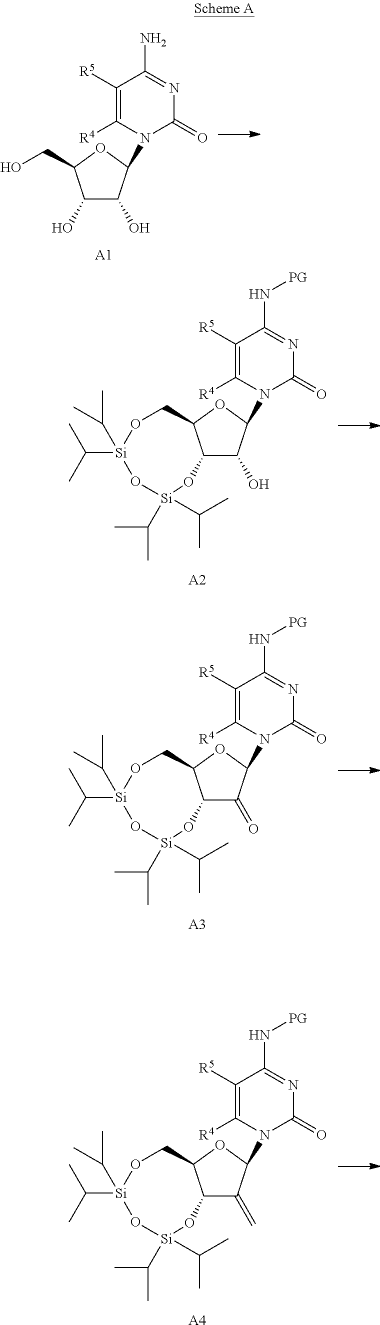

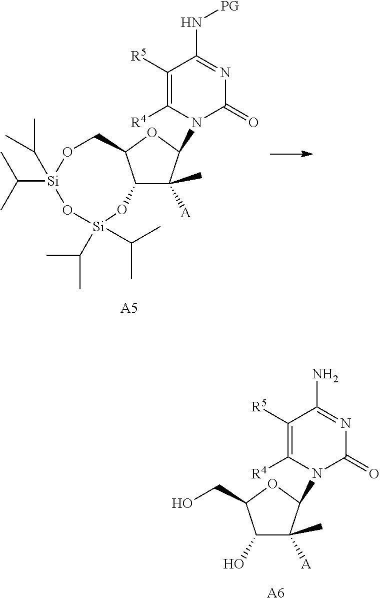

Wherein PG is a protecting group and A, R⁴ and R⁵ are as defined above for the Compounds of Formula (I).

Nucleoside compounds of formula A1 can be bis-protected at the ribose 3′ and 5′ positions using the tetraisopropyldisiloxanyi group to provide compounds of formula A2. Compounds of formula A2 are then oxidized, using for example, the Dess-Martin Periodinane, to provide the 2′-ketone of formula A3. Wittig olefination with methyltriphenylphosphonium bromide/potassium hexamethyldisilazide provides compounds of formula A4. The olefin moiety of the compounds of formula A4 can be manipulated using methods well-known to those skilled in the art of organic synthesis to provide the 2′-substituted compounds of formula A5. The silyl protecting group is then removed using tetrabutylammonium fluoride and the cytidine protecting group is then removed with methanolic ammonia to provide the compounds of formula A6.

Scheme B shows a method useful for making nucleoside compounds of formula B6, which correspond to the Compounds of Formula (I), wherein X is O; R¹ and R² are each H; R³ is methyl and B is:

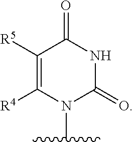

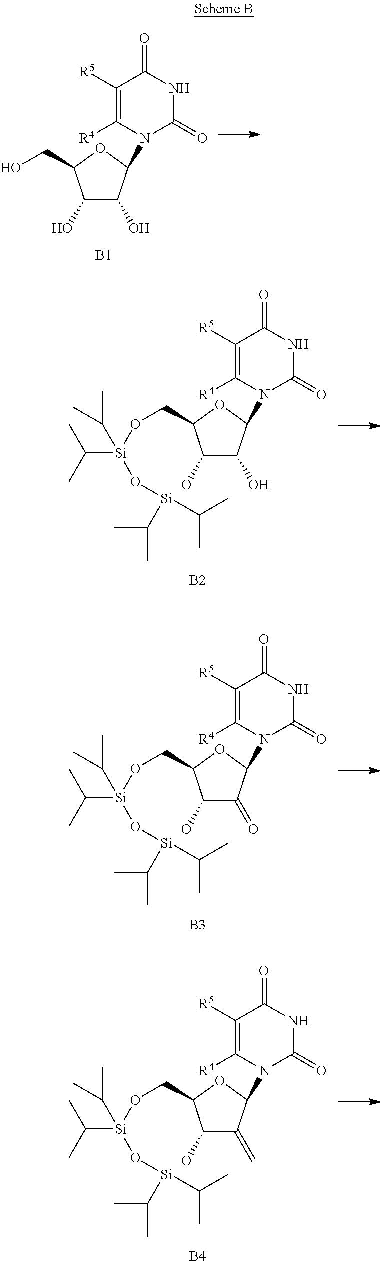

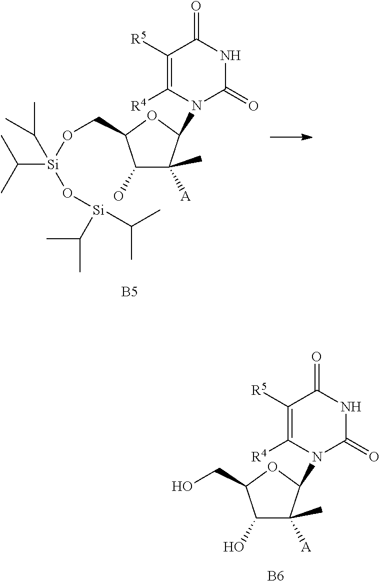

Wherein A, R⁴ and R⁵ are as defined above for the Compounds of Formula (I).

Compounds of formula B1 were protected at the ribose 3′ and 5′ positions using the tetraisopropyldisiloxanyl group to provide compounds of formula B2. Compounds of formula B2 were treated with Dess-Martin Periodinane to provide the desired 2′-ketone of formula B3. Wittig olefination with methyltriphenylphosphonium bromide/potassium hexamethyldisilazide provided compounds of formula B4. The olefin moiety of the compounds of formula B4 can be manipulated using methods well-known to those skilled in the art of organic synthesis to provide the 2′-substituted compounds of formula B5. The tetraisopropyldisiloxanyl protecting group is then removed using tetrabutylammonium fluoride to provide the compounds of formula A6.

Scheme C shows a method useful for making nucleoside compounds of formula C9, which correspond to the Compounds of Formula (I), wherein X is O; R¹ and R² are each H; R³ is methyl and B is:

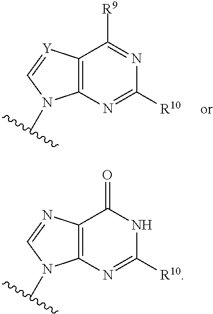

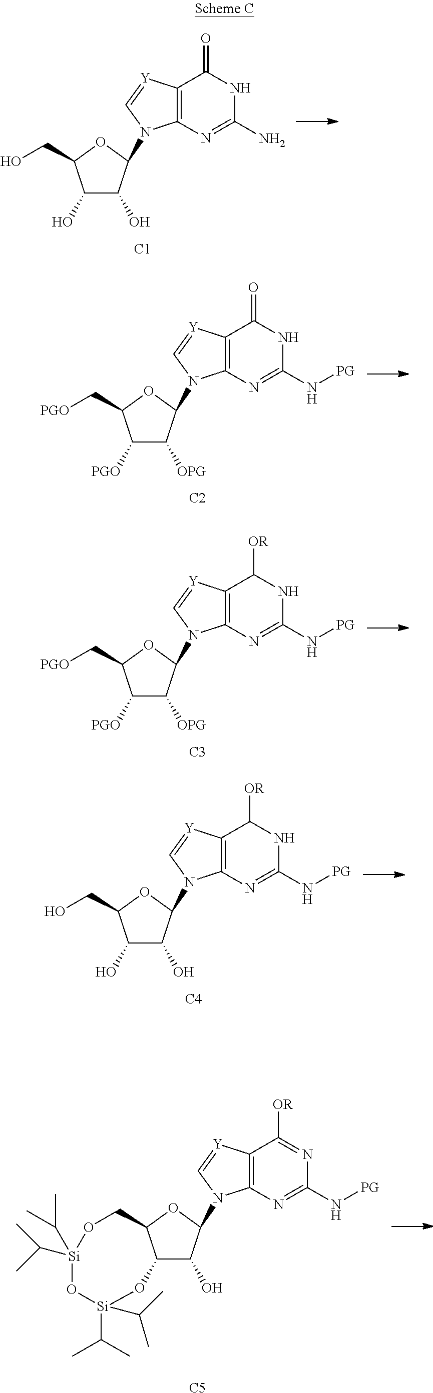

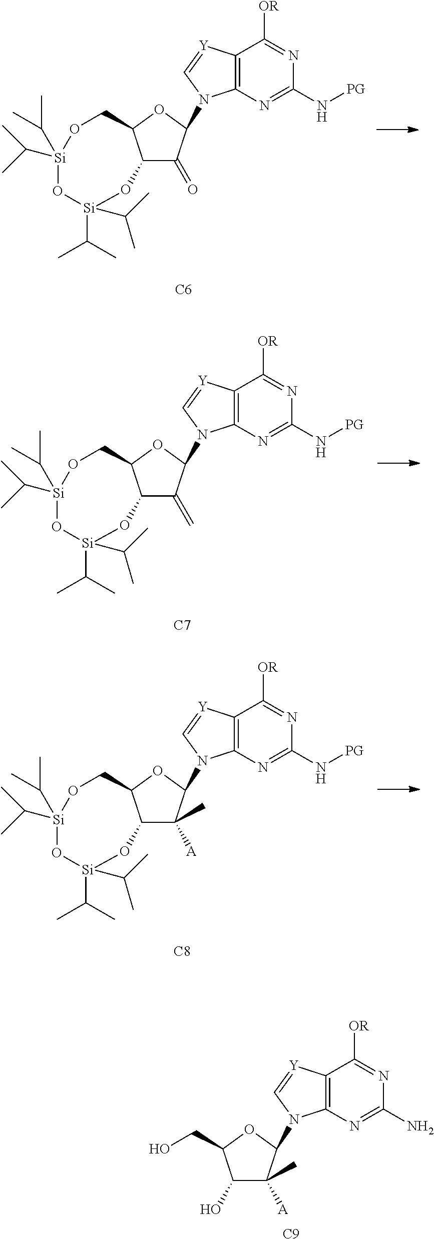

Wherein PG is a protecting group and R⁴ and R⁵ are as defined above for the Compounds of Formula (I).

A compound of formula C1 can have all of its hydroxyl groups protected using well known methods to provide the compounds of formula C2. A compound of formula C2 can then be subjected to Mitsunobu conditions to provide purine ethers of formula C3. The hydroxyl groups of C3 are then deprotecting to provide the compounds of formula C4, which can subsequently have their 3′-OH and 5′-OH protected using the tetraisopropyldisiloxanyl group to provide the compounds of formula C5. Compounds of formula C5 are then oxidized, using for example, the Dess-Martin Periodinane, to provide the 2′-ketone compounds of formula C6. Wittig olefination with methyltriphenylphosphonium bromide/potassium hexamethyldisilazide provides the compounds of formula C7. The olefin moiety of the compounds of formula C7 can be manipulated using methods well-known to those skilled in the art of organic synthesis to provide the 2′-substituted compounds of formula C8. The tetraisopropyldisiloxanyl protecting group is then removed using tetrabutylammonium fluoride, followed by deprotection of the protected aryl amine group to provide the compounds of formula C9.

Scheme D shows a method useful for making nucleoside compounds of formula D3, which correspond to the Compounds of Formula (I), wherein X is O; R² is H; R³ is methyl; B is as defined above for the Compounds of Formula (I); and R¹ is:

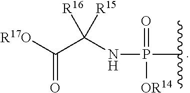

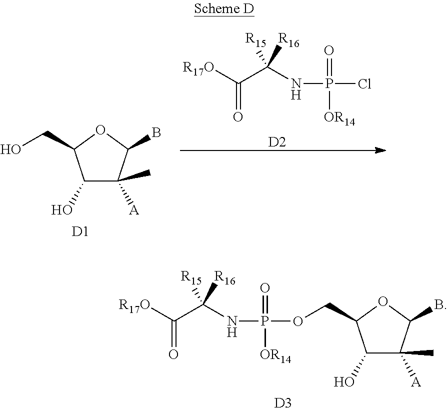

Wherein B, R¹⁴, R¹⁵, R¹⁶ and R¹⁷ are as defined above for the Compounds of Formula (I).

A compound of formula D1 can be coupled with a compound of formula D2 (compounds of formula D2 can be synthesized using methods described in U.S. Pat. No. 7,879,815) in the presence of either t-butylmagnesium bromide or N-methylimidazole to provide the compounds of formula D3.

Scheme G shows a method useful for making nucleoside compounds of formula G3, which correspond to the Compounds of Formula (I) wherein A is —N(R²⁰)₂, —NHSO₂—(C₁-C₆ alkyl), —NHC(O)N(R²⁰)₂, —NHOH, —NHC(O)R²⁰ or —NHC(O)OR²⁰.

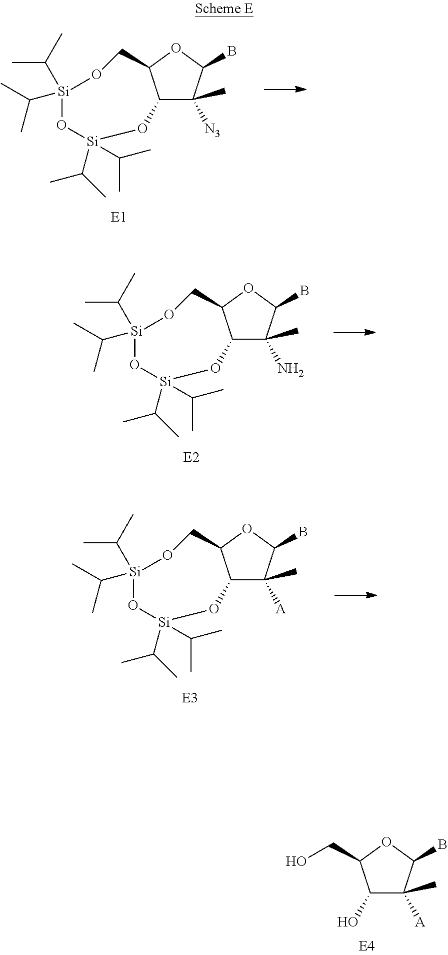

Wherein A is —N(R²⁰)₂, —NHSO₂—(C₁-C₆ alkyl), —NHC(O)N(R²⁰)₂, —NHOH, —NHC(O)R²⁰ or —NHC(O)OR²⁰, and R²⁰ and B are as defined above for the Compounds of Formula (I).

An azide compound of formula E1 can be hydrogenated using for example, palladium catalysis in the presence of hydrogen gas, to provide the corresponding amine derivatives of formula E2. The amine group of a compound of formula E2 can then converted to a variety of functional groups using methods and reagents (such as acyl chlorides, isocyanates and chloroformates) well-known to those skilled in the art of organic synthesis to provide the compound of formula E3. These compounds were then silyl deprotected using tetrabutylammonium fluoride to provide the compounds of type E4, which correspond to the Compounds of Formula (I) wherein A is —N(R²⁰)₂, —NHSO₂—(C₁-C₆ alkyl), —NHC(O)N(R²⁰)₂, —NHOH, —NHC(O)R²⁰ or —NHC(O)OR²⁰.

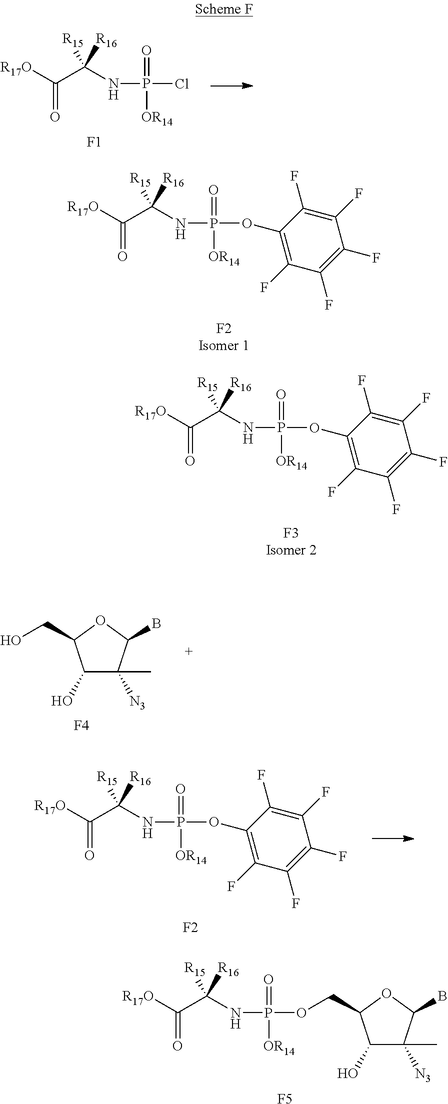

Wherein B, R¹⁴, R¹⁵, R¹⁶ and R¹⁷ are as defined above for the Compounds of Formula (I).

A compound F1 can be reacted with pentafluoro phenol to produce two isomers F2 and F3. These isomers can be individually reacted with F4 to produce compounds of structure F5.

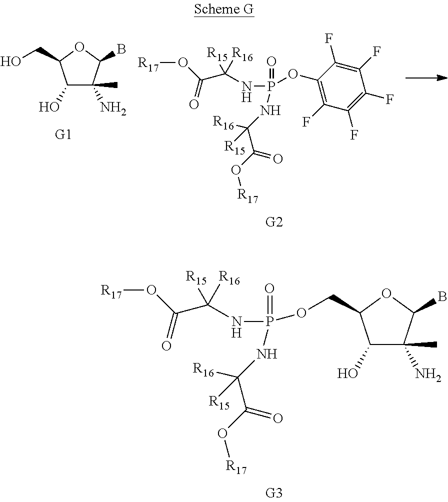

Wherein B, R¹⁵, R¹⁶, R¹⁷ and R²⁰ are as defined above for the Compounds of Formula (I).

Compounds of type G1 can react with compounds of type G2 to provide compounds of type G3.

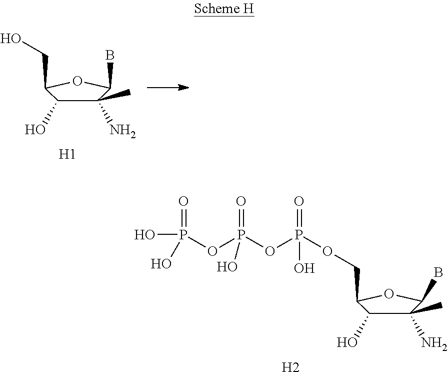

Wherein B is as defined above for the Compounds of Formula (I).

Nucleosides such as H1 (synthesis described in example G) can be converted to the triphosphate H2 by treatment with phosphorus oxychloride followed by pyrophosphate.

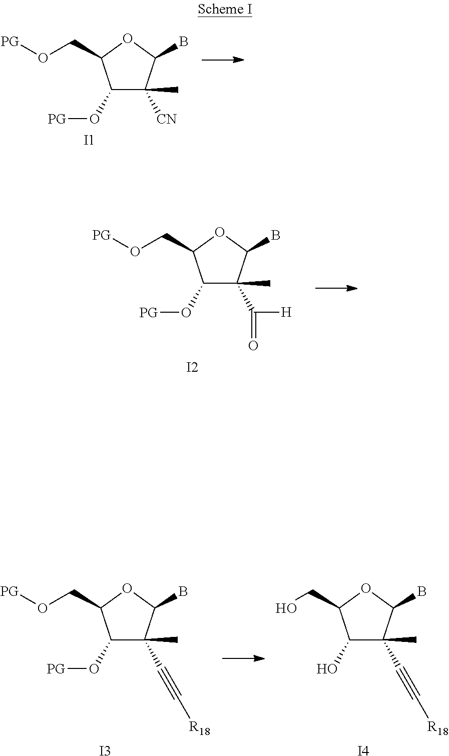

Wherein B and R¹⁸ are as defined above for the Compounds of Formula (I).

Compounds of type I1 (synthesis described in Scheme P) can be treated with a variety of hydride sources to provide compound II. I1 can be converted to the alkyne I3 which can then be globally deprotected to provide I4.

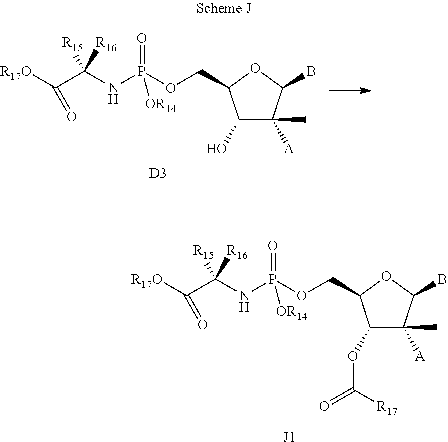

Wherein B, R¹⁴, R¹⁵, R¹⁶ and R¹⁷ are as defined above for the Compounds of Formula (I).

Compounds of type J1 can be synthesized using standard coupling reagents such as acyl/aryl chlorides.

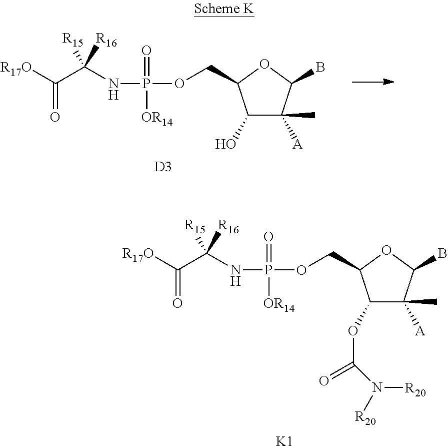

Wherein B, R¹⁴, R¹⁵, R¹⁶ R¹⁷ and R²⁰ are as defined above for the Compounds of Formula (I).

Compounds of type K1 can be synthesized using coupling reagents such as carbamyl chlorides.

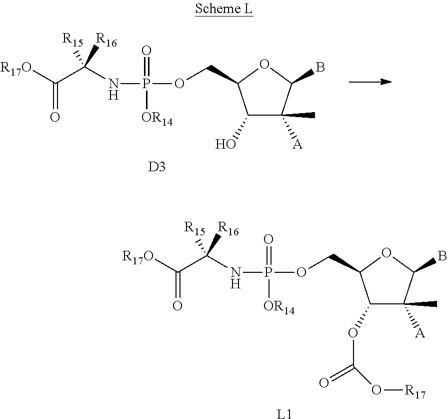

Wherein B, R¹⁴, R¹⁵, R¹⁶ and R¹⁷ are as defined above for the Compounds of Formula (I).

Compounds of type L1 can be synthesized using standard coupling reagents such as alkyl/aryl chloroformates.

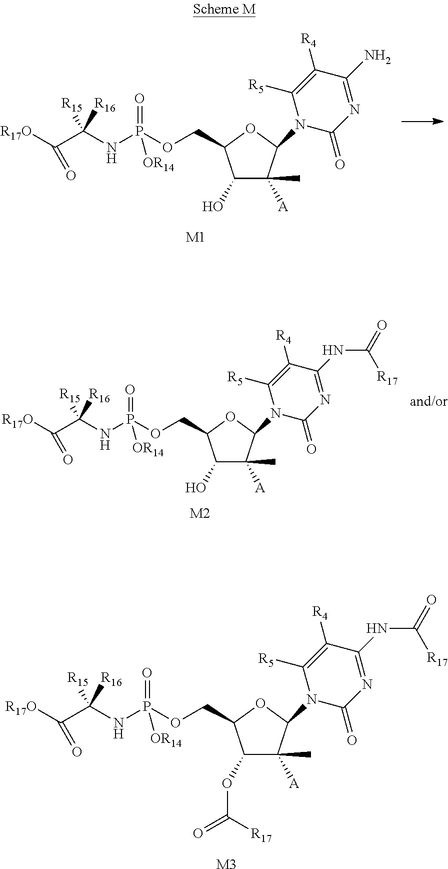

Wherein A, B, R⁴, R⁵, R¹⁴, R¹⁵, R¹⁶ and R¹⁷ are as defined above for the Compounds of Formula (I).

Compounds of type M2 and M3 can be synthesized from M1 using standard coupling reagents such as acyl/aryl chlorides in an aprotic solvent such as methylene chloride.

Compounds of formula A6, B6, C9, D3, E4, F5, G3, H2, I4, J1, K1, L1, and M3 may be further elaborated using methods that are be well-known to those skilled in the art of organic synthesis or, for example, the methods described in the Examples below, to make the full scope of the Compounds of Formula (I).

One skilled in the art of organic synthesis will recognize that the synthesis of compounds with multiple reactive functional groups, such as —OH and NH₂, may require protection of certain functional groups (i.e., derivatization for the purpose of chemical compatibility with a particular reaction condition). Suitable protecting groups for the various functional groups of these compounds and methods for their installation and removal are well known in the art of organic chemistry. A summary of many of these methods can be found in Greene et al, Protective Groups in Organic Synthesis (1991) Wiley, New York.

One skilled in the art of organic synthesis will also recognize that one route for the synthesis of the Compounds of Formula (I) may be more desirable depending on the choice of appendage substituents. Additionally, one skilled in the relevant art will recognize that in some cases the order of reactions may differ from that presented herein to avoid functional group incompatibilities and thus adjust the synthetic route accordingly.

The starting materials used and the intermediates prepared using the methods set forth in Schemes A-M may be isolated and purified if desired using conventional techniques, including but not limited to filtration, distillation, crystallization, chromatography and alike. Such materials can be characterized using conventional means, including physical constants and spectral data.

Non-limiting examples of the Compounds of Formula (I) include compounds 1-36 as set forth in the Examples below, and pharmaceutically acceptable salts thereof.

### EXAMPLES

### General Methods

Solvents, reagents, and intermediates that are commercially available were used as received. Reagents and intermediates that are not commercially available were prepared in the manner as described below. ¹H NMR spectra were obtained on a Varian VNMR System 400 (400 MHz) and are reported as ppm downfield from Me₄Si with number of protons, multiplicities, and coupling constants in Hertz indicated parenthetically. Where LC/MS data are presented, analyses was performed using an Agilent 6110A MSD or an Applied Biosystems API-100 mass spectrometer and Shimadzu SCL-10A LC column: Altech platinum C18, 3 micron, 33 mm×7 mm ID; gradient flow: 0 minutes—10% CH₃CN, 5 minutes—95% CH₃CN, 5-7 minutes—95% CH₃CN, 7 minutes—stop. The parent ion is given. Flash chromatography on silica gel was performed using pre-packed normal phase silica from Isco, Biotage, Inc. or bulk silica from Fisher Scientific. Unless otherwise indicated, flash chromatography on silica gel was performed using a gradient elution of hexanes/ethyl acetate, from 100% hexanes to 100% ethyl acetate.

### Example 1

### Preparation of Intermediate Compound Int-1c

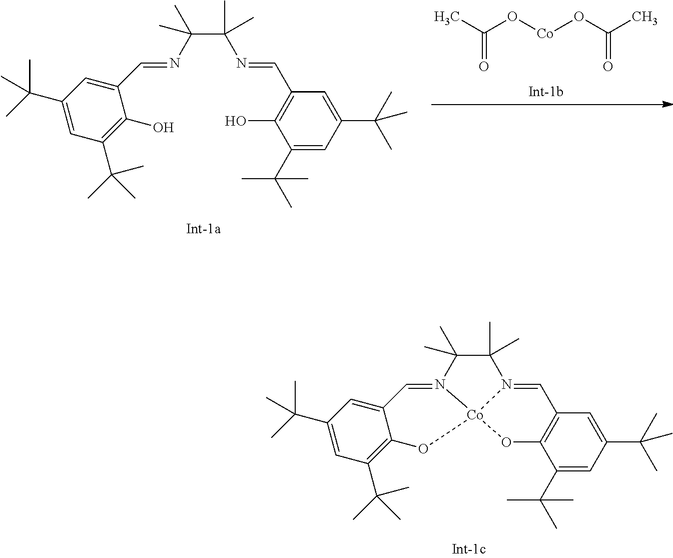

Compound Int-1a (733 mg, 1.33 mmol) was suspended in ethanol (10 mL) and the resulting suspension was heated to 80° C. and allowed to stir for 5 minutes. Compound Int-1b (236 mg, 1.33 mmol) was then added and the resulting reaction was allowed to stir at 80° C. for an additional 2 hours. The reaction was then cooled to room temperature using in an ice bath and the reaction mixture was filtered. The collected red solid was dried under vacuum to provide compound Int-1c (579 mg, 72%).

### Example 2

### Preparation of Intermediate Compound Int-2e

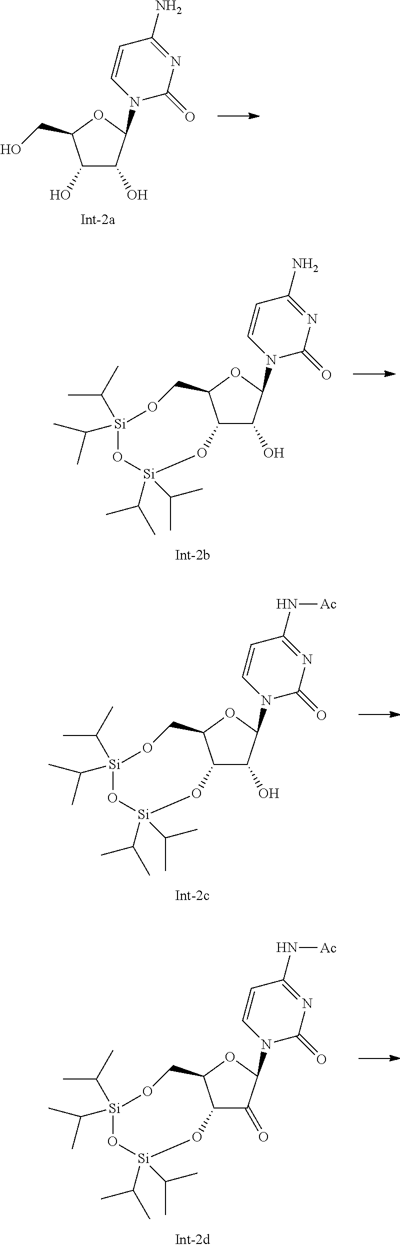

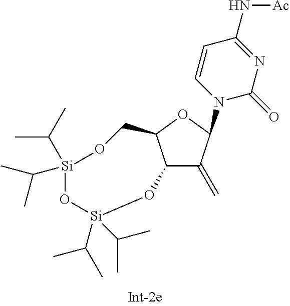

### Step A—Synthesis of Compound Int-2b

Cytidine (Int-2a, 8.0 g, 32.89 mmol) was azeotroped with pyridine (2×15 mL) and then suspended in pyridine (25 mL). To the suspension was added tetraisopropyldisiloxanedichloride (12.0 mL, 35.4 mmol) dropwise over fifteen minutes and the resulting reaction was allowed to stir for about 15 hours at room temperature. The reaction mixture was diluted with water and extracted with ethyl acetate. The organic extract were washed with brine, dried over sodium sulfate, filtered and concentrated in vacuo. The residue obtained was triturated with heptane to provide 13.5 g of compound Int-2b as a white solid [M+H]=486.5.

### Step B—Synthesis of Compound Int-2c

Compound Int-2b (13.5 mmol, 27.8 mmol) was dissolved in ethanol (200 mL) and treated with acetic anhydride (10 mL). The resulting reaction was heated to 65° C. and allowed to stir at this temperature for 3 hours, then the reaction mixture was concentrated in vacuo. The residue obtained was cooled to 0° C. in an ice bath and treated with saturated sodium bicarbonate, then extracted with ethyl acetate. The organic extract was dried over sodium sulfate, filtered and concentrated in vacuo to provide 14.5 g of compound Int-2c, which was used without further purification. [M+H]=528.6

### Step C—Synthesis of Compound Int-2d

A solution of compound Int-2c (8.0 g, 15.15 mmol) in methylene chloride (120 mL) was cooled to 0° C., then the Dess Martin Periodinane (15 g, 34.3 mmol) was added. The resulting reaction was allowed to stir for about 15 hours at room temperature and was then diluted with diethyl ether (400 mL). The resulting solution was washed with a mixture of saturated sodium bicarbonate and 10% sodium thiosulfate (1:1). The organic phase was collected and was dried over sodium sulfate, filtered and concentrated in vacuo to provide 7.8 g of compound Int-2d, which was used without purification. [M+H]=526.

### Step D—Synthesis of Compound Int-2e

Methyltriphenylphosphonium bromide (2.72 g, 7.6 mmol) was diluted with tetrahydrofuran (25 mL) and to the resulting suspension was added KHMDS (0.5 M, 14.5 mL, 7.22 mmol). The resulting reaction was allowed to stir at room temperature for 20 minutes, then the reaction was cooled to 0° C. using an ice bath and a solution of compound Int-2d (1.0 g, 1.9 mmol) in tetrahydrofuran (5 mL) was added dropwise. The resulting reaction was warmed to room temperature and stirred for 4 hours, then the reaction was quenched with saturated ammonium chloride and extracted with ethyl acetate. The organic extract was washed with brine, dried over sodium sulfate, filtered and concentrated in vacuo. The resulting residue was purified using flash chromatography on silica gel (2:1 hexanes/ethyl acetate) to provide 750 mg of compound Int-2e. [M+H]=524.5

### Example 3

### Preparation of Intermediate Compound Int-3d

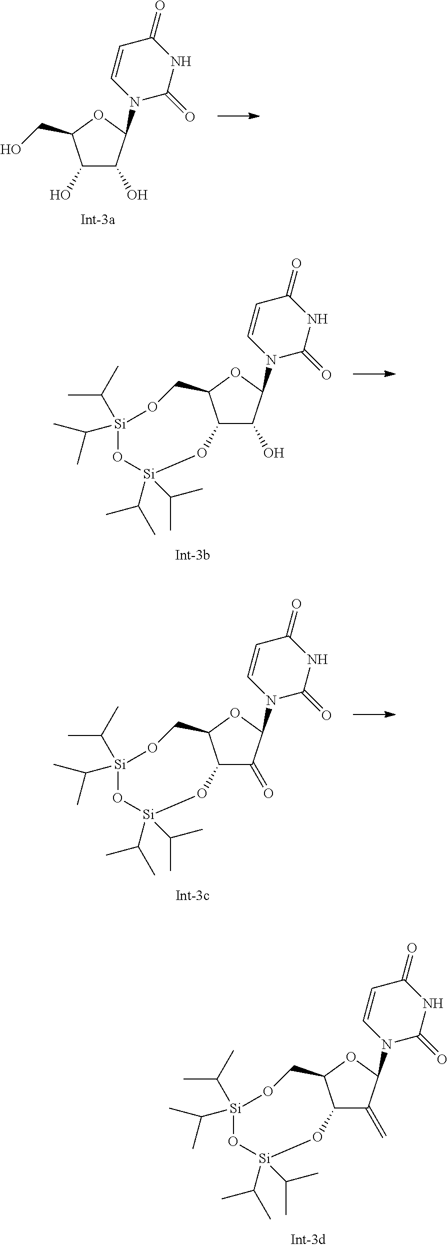

### Step A—Synthesis of Compound Int-3b

Uridine (Int-3a, 5.0 g, 18.0 mmol) was azeotroped with pyridine (2×15 mL) and then suspended in pyridine (25 mL). Tetraisopropyldisiloxanedichloride (6.06 mL mL, 18.95 mmol) was added dropwise over fifteen minutes and the reaction was allowed to stir for about 15 hours at room temperature. The reaction was diluted with water and extracted with ethyl acetate. The organic layers were washed with brine, dried over sodium sulfate, and concentrated in vacuo. The residue was azeotroped with toluene (2×50 mL) to provide 7.8 g of compound Int-3b as a white solid that was used without purification. [M+H]=487.42.

### Step B—Synthesis of Compound Int-3c

Using the method described in Example 2, Step C, compound Int-3b was converted to compound Int-3b (3.8 g). [M+H]=485.2

### Step C—Synthesis of Compound Int-3d

Using the method described in Example 2, Step D, compound Int-3c was converted to compound Int-3d (750 mg) [M+H]=505.2.

### Example 4

### Preparation of Compounds 1 and 2

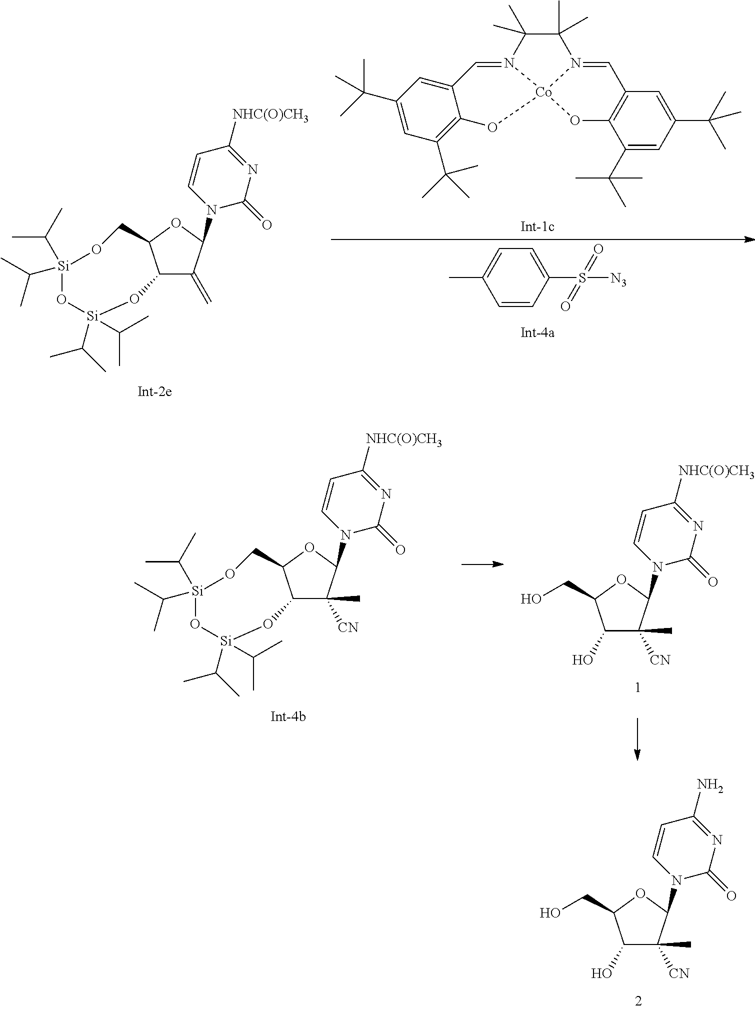

### Step A—Synthesis of Compound Int-4b

Compound Int-2e (300 mg, 0.57 mmol), Compound Int-1c (10 mg), and Compound Int-4a (3.1 g, 17.1 mmol) were dissolved in dioxane (2 mL) and the resulting reaction was allowed to stir for 5 minutes at room temperature. A solution of phenylsilane (68 mg, 0.63 mmol) in ethanol (1 mL) was then added dropwise over 2 minutes and the reaction was allowed to stir for and additional 30 minutes. The reaction was then quenched with brine and extracted with ethyl acetate. The organic extract was washed with brine, dried over sodium sulfate, filtered and concentrated in vacuo. The residue obtained was purified using flash chromatography on silica gel (2:1 hexanes/ethyl acetate) to provide compound Int-4b (80 mg). [M+H]=551.5

### Step B—Synthesis of Compound 1

Compound Int-4b (23 mg, 0.042 mmol) was dissolved in tetrahydrofuran (1 mL) and to the resulting solution was added TBAF (1.0 M, 0.1 mmol). The reaction was allowed to stir for 1 hour, then the reaction mixture was concentrated in vacuo and the crude residue was purified using flash chromatography on silica gel (20% methanol in methylene chloride) to provide 5.0 mg of compound 1.

### Step C—Synthesis of Compound 2

Compound 1 (3.8 mg, 0.012 mmol) was dissolved in 7M ammonia in methanol (2 mL) and stirred for 3 hours at room temperature. The reaction was concentrated in vacuo and the residue was purified using flash chromatography on silica gel (25% methanol in methylene chloride) to provide 3.0 mg of compound 2. ¹H-NMR (400 MHz, CD₃OD): δ: 8.11 (d, 1H, J=7.58 Hz), 6.38 (s, 1H), 5.90 (bs, 1H), 4.0 (m, 3H), 3.8 (m, 1H), 1.27 (s, 3H).

The following compounds of the present invention were made using the methods described in the Example above and substituting the appropriate reactants and/or reagents.

| Compound | | |
|---|---|---|
| No. | Structure | MS (M + H) |
| 3 | 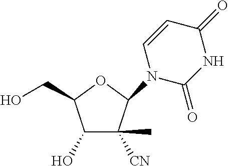 [SMILES: C[C@@]1(C#N)[C@H](O)[C@@H](CO)O[C@H]1n1ccc(=O)[nH]c1=O] | 290.2 |

### Compound 3 ¹H-NMR (400 MHz, CD₃OD): δ: 8.13 (d, 1H, J=8.0 Hz), 6.33 (s, 1H), 5.7 (d, 1H, J=8.0 Hz), 4.06 (m, 1H), 4.0-3.95 (m, 2H), 3.79 (m, 1H), 1.33 (s, 3H).

### Example 5

### Preparation of Intermediate Compound Int-5b

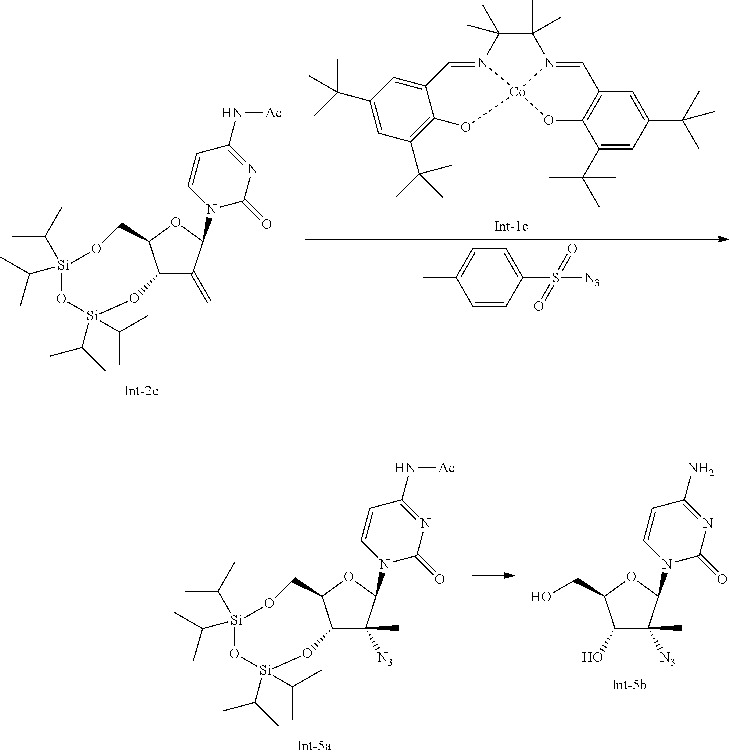

### Step A—Synthesis of Compound Int-5a

Compound Int-2e (500 mg, 0.955 mmol) and Compound Int-1c (20 mg, 0.033 mmol) were dissolved in tosyl azide (3.0 g, 15.01 mmol) and the resulting mixture was allowed to stir for 5 minutes at room temperature. A solution of phenylsilane (121 mg, 1.11 mmol) in ethanol (3 mL) was added dropwise over 2 minutes and the reaction was allowed to stir for an additional 30 minutes. The reaction was then quenched with brine and extracted with ethyl acetate. The organic extract was washed with brine, dried over sodium sulfate, filtered and concentrated in vacuo. The resulting residue was purified using flash column chromatography on silica gel (2:1 hexanes/ethyl acetate) to provide 197 mg of compound Int-5a. [M+H]=567.2

### Step B—Synthesis of Compound Int-5b

Compound Int-5a (75 mg, 0.132 mmol) was dissolved in tetrahydrofuran (1 mL) and treated with 1.0M tetrabutylammonium fluoride (0.265 mmol). The reaction was allowed to stir for 1 hour and then the reaction mixture was concentrated in vacuo. The residue obtained was dissolved in 7M ammonia in methanol (2 mL) and allowed to stir for 3 hours at room temperature. The reaction was concentrated in vacuo and the residue obtained was purified using flash column chromatography on silica gel (20% methanol in dichloromethane) to provide 11 mg of compound Int-5b.

### Example 6

### Preparation of Compound Int-6b

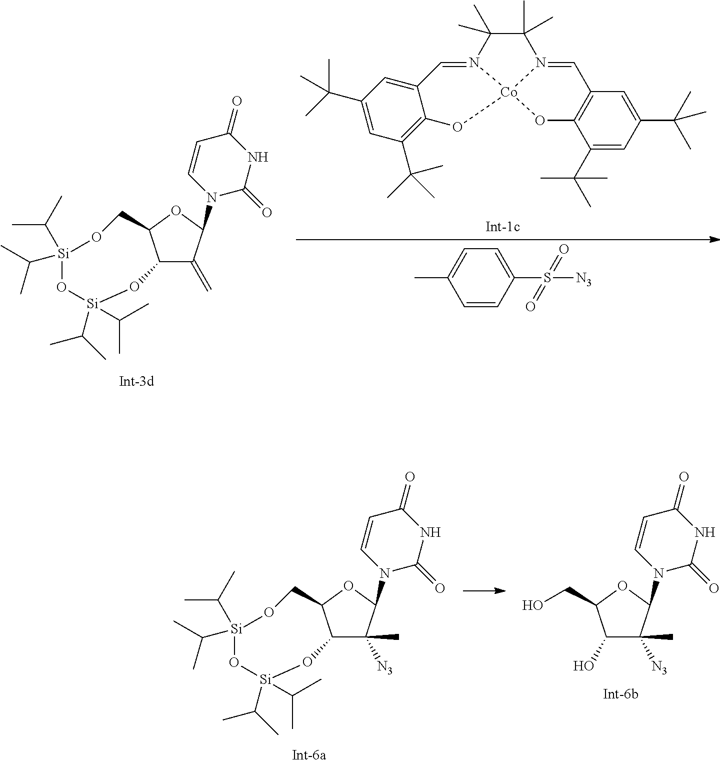

### Step A—Synthesis of Compound Int-6a

A solution of compound Int-3d (5.0 g, 10.36 mmol), compound Int-1c (125 mg, 0.207 mmol) and tosyl azide (24 g, 122 mmol) was allowed to stir for 5 minutes, then a solution of phenylsilane (1.34 g, 12.43 mmol) in ethanol (25 mL) was added dropwise over 30 minutes. The resulting reaction was allowed to stir for 30 minutes, then the reaction was quenched with brine and extracted with ethyl acetate. The organic extract was washed with brine, dried over sodium sulfate, filtered and concentrated in vacuo. The resulting residue was purified using flash column chromatography on silica gel (4:1 hexanes/ethyl acetate) to provide 2.5 g of compound Int-6a. [M+H]=526.2

### Step B—Synthesis of Compound 6b

To a solution of compound Int-6a (1.6 g, 3.04 mmol) in tetrahydrofuran (35 mL) was added tetrabutylammonium fluoride (1.0 M, 6.09 mmol). The resulting reaction was allowed to stir for 1 hour, then the reaction mixture was concentrated in vacuo. The residue obtained was purified using flash column chromatography on silica gel (10% methanol in dichloromethane) to provide 650 mg of compound 6b.

### Example 7

### Preparation of Compound 4

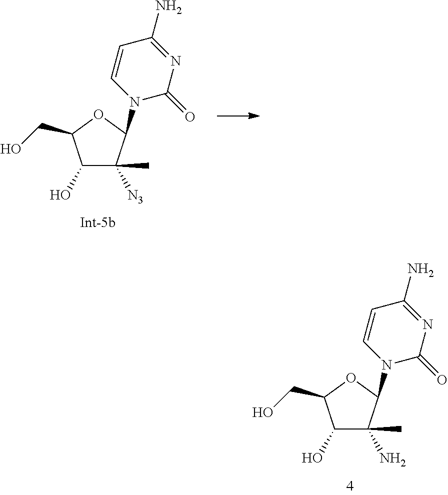 [SMILES: C[C@@]1(N=[N+]=[N-])[C@H](O)[C@@H](CO)O[C@H]1n1ccc(N)nc1=O ; C[C@@]1(N)[C@H](O)[C@@H](CO)O[C@H]1n1ccc(N)nc1=O]

Compound Int-5b (10 mg, 0.035 mmol) was dissolved in methanol (10 mL) and to the resulting solution was added 10% palladium on carbon (100 mg). The resulting reaction was evacuated and placed under hydrogen atmosphere (using a hydrogen filled balloon) and allowed to stir for 2 hours. The reaction mixture was then filtered through a short pad of celite and the filtrate was concentrated in vacuo to provide 9.5 mg of compound 4, which was used without further purification. [M+H]=257.3.

The following compounds of the present invention were made using the methods described in the Example above and substituting the appropriate reactants and/or reagents.

| Compound | | Starting | MS |
|---|---|---|---|
| No. | Structure | Material | (M + H) |
| 5 | 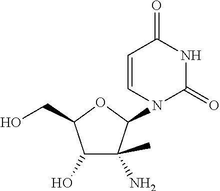 | Int-6b | 258.0 |

### Example 8

### Preparation of Compounds Int-8b and Int-8c

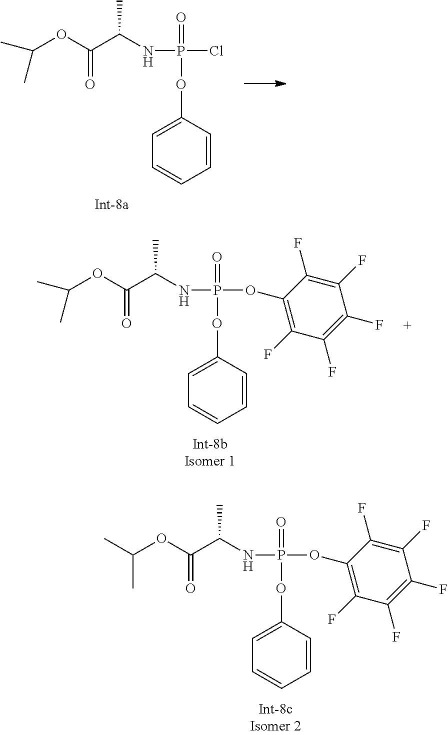 [SMILES: CC(C)OC(=O)[C@H](C)NP(=O)(Cl)Oc1ccccc1 ; CC(C)OC(=O)[C@H](C)NP(=O)(Oc1ccccc1)Oc1c(F)c(F)c(F)c(F)c1F ; CC(C)OC(=O)[C@H](C)NP(=O)(Oc1ccccc1)Oc1c(F)c(F)c(F)c(F)c1F]

A stirred solution of Int-8a (14.2 g, 46.4 mmol) in dichloromethane (112 mL) was treated with pentafluorophenol (8.55 g, 46.4 mmol) in one portion. The solution was cooled to 0° C. and triethylamine (6.47 mL, 46.4 mmol) was added dropwise under nitrogen. The reaction was stirred overnight at room temperature. LCMS shows mainly product. The reaction mixture was washed with water (100 mL), dried over Na₂SO₄, filtered and concentrated in vacuo. The residue obtained was purified by silica gel column chromatography (0-30% hexanes/ethyl acetate) to provide 12.56 g of a white solid. The solid was recrystallized using 10% MBTE/hexanes to provide compound Int 8b (6.15 g) as a white solid. The mother liquor (5.3 g of a 4:1 mixture isomer 2/isomer 1) was purified by silica gel chromatography (1:1 hexanes/ethyl acetate) to provide Int 8c (4.04 g) and additional Int 8b (805 mg).

Phosphorylamino chloride reactants of type Int-8a can be synthesized using the methods described in U.S. Pat. No. 7,879,815.

### Example 9

### Preparation of Compound 6

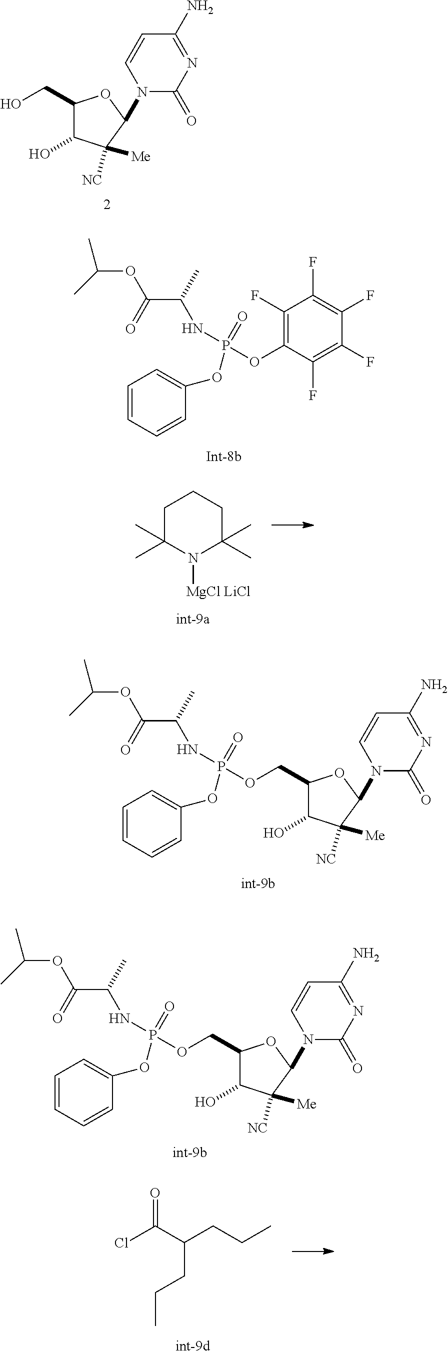

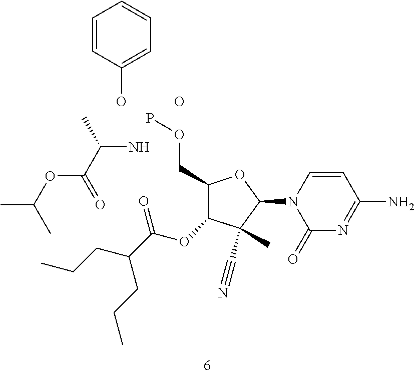

### Step A—Synthesis of Compound Int-9b

NMP (1.0 ml) was added to a solution of compound 2 (50 mg, 0.188 mmol) in THF (3 ml). To the resulting incomplete clear solution was added dropwise int-9a (0.188 ml, 0.188 mmol) at 0° C. The reaction was stirred for 5 minutes at 0° C., then was treated dropwise with a solution of int-8b (85 mg, 0.188 mmol) in 0.5 ml of THF. The ice bath was removed and the reaction suspension was stirred at room temperature for 1.5 hour. The reaction was quenched the reaction with 10 ml of sat. NH₄Cl and then diluted in 50 ml of EtOAc and washed with 2×20 ml of water and 20 ml of brine. The organic layer was dried over Na₂SO₄, filtered and concentrated in vacuo. The crude material was purified using flash column chromatography on a 12 g silica gel column (2.5-10% MeOH/CH₂Cl₂) to provide the title compound int-9b, (46 mg, 45.7%) as white solid. (ES, m/z): 535.87 [M+H], ¹H -NMR (CD₃OD, 500 MHz, ppm): δ 7.66 (d, 1H, J=8 Hz), 7.38 (t, 2H, J=8 Hz), 7.27 (t, 2H, J=8 Hz), 7.22 (t, 1H, J=8 Hz), 6.38 (s, 1H), 5.86 (d, 1H, J=8 Hz), 4.94-5.00 (m, 1H), 4.53 (ddd, 1H, J=10, 6, 2 Hz), 4.38 (ddd, 1H, J=10, 6, 4 Hz), 4.09-4.13 (m, 1H), 3.97 (d, 1H, J=10 Hz), 1.36 (d, 3H, J=8 Hz), 1.23 (d, 6H, J=8 Hz).

### Step B—Synthesis of Compound 6

To a solution of int-9b (32 mg, 0.060 mmol) and DMAP (0.730 mg, 5.98 μmol) in THF (1.5 ml) was added triethylamine (0.025 ml, 0.179 mmol) and water (1.0 ml) at 0° C. The mixture was stirred at 0° C. for 5 minutes, and then int-9d (0.026 ml, 0.149 mmol) was added to the reaction mixture slowly and the reaction was stirred at 0° C. for 0.5 hour. The reaction was quenched with saturated NH₄Cl (4.0 ml) and extracted with 2×20 ml of EtOAc. The organic layers were washed with brine (20 ml), dried over Na₂SO₄, filtered and concentrated in vacuo. The crude material was purified on prep TLC eluting with 10% MeOH/CH₂Cl₂ to yield the title compound 6, (30 mg, 76%) as white solid. (ES, m/z): 662.01 [M+H], ¹H-NMR (CD₃OD, 500 MHz, ppm): δ 7.65 (d, 1H, J=8 Hz), 7.36-7.41 (m, 2H), 7.27 (d, 2H, J=8 Hz), 7.22 (t, 1H, J=8 Hz), 6.37 (s, 1H), 5.86 (d, 1H, J=8 Hz), 5.17 (d, 1H, J=8 Hz), 4.95-5.02 (m, 1H), 4.48-4.74 (m, 1H), 4.26-4.33 (m, 2H), 3.89-3.99 (m, 1H), 2.50-2.58 (m, 1H), 1.64-1.75 (m, 2H), 1.41-1.57 (m, 2H), 1.31-1.42 (m, 9H), 1.19-1.26 (m, 6H), 0.49-0.56 (m, 4H).

The following compounds of the present invention were made using the methods described in the Example above and substituting the appropriate reactants and/or reagents.

| Compound | | Starting | |
|---|---|---|---|
| No. | Structure | Material | MS (M + H) |
| 7 | 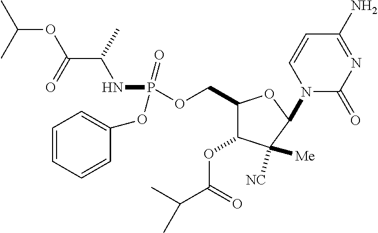 | Int-9b | 606.2 |
| 8 | 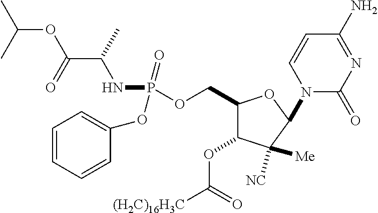 | Int-9b | 802.44 |

### Example 10

### Preparation of Compound 9

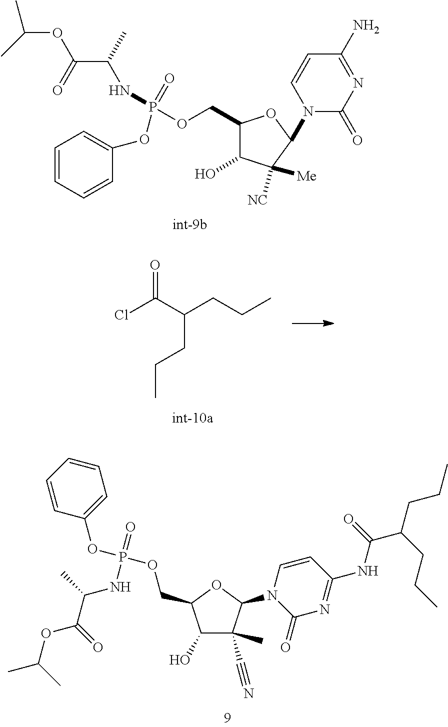

To a solution of int-9b (50 mg, 0.093 mmol) in CH₂Cl₂ (2 ml) was added DIPEA (0.082 ml, 0.467 mmol) followed by addition of int-1e (0.016 ml, 0.093 mmol) at room temperature. The reaction was stirred for 3 days, then was diluted with 40 ml of CH₂Cl₂ and washed with brine (20 mL), dried ober sodium sulfate, filtered and concentrated in vacuo. The crude material was purified using flash column eluting with 0-5% MeOH/CH₂Cl₂ to isolated the title compound 9 as white solid (10.8 mg, 17.4%). (ES, m/z): 662.06 [M+H], ¹H-NMR (CD₃OD, 500 MHz, ppm): δ 8.05 (d, 1H, J=8 Hz), 7.50 (d, 1H, J=8 Hz), 7.38 (t, 2H, J=8 Hz), 7.28 (d, 2H, J=8 Hz), 7.20 (t, 1H, J=8 Hz), 4.84-5.02 (m, 1H), 5.54 (ddd, 1H, J=10, 6, 2 Hz), 4.41 (ddd, 1H, J=10, 6, 4 Hz), 4.15-4.19 (m, 1H), 4.01 (d, 1H, J=8 Hz), 3.90-3.98 (m, 1H), 2.50-2.58 (m, 1H), 1.60-1.69 (m, 2H), 1.42-1.51 (m, 2H), 1.20-1.40 (m, 16H), 0.91 (d, 3H, J=8 Hz).

The following compounds of the present invention were made using the methods described in the Example above and substituting the appropriate reactants and/or reagents.

| Compound | | Starting | |
|---|---|---|---|
| No. | Structure | Material | MS (M + H) |
| 10 | 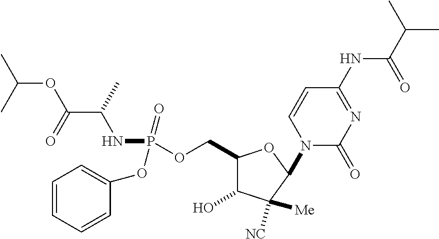 | Int-9b | 605.92 |
| 11 | 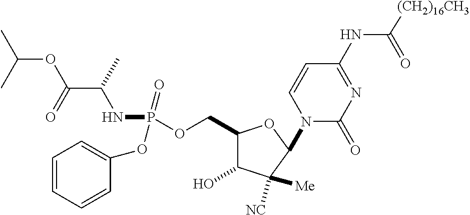 | Int-9b | 802.57 |

### Example 11

### Preparation of Compound 12

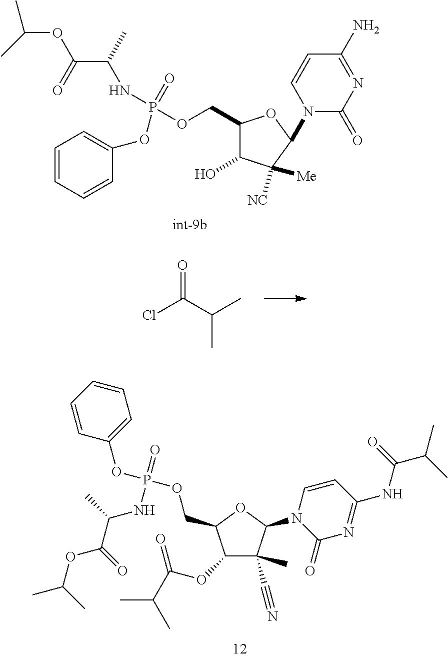

To a solution of int-9b (150 mg, 0.280 mmol) in CH₂Cl₂ (2 ml) was added DIPEA (0.245 ml, 1.401 mmol) and dimethylacetyl chloride (0.032 ml, 0.308 mmol) at room temperature. The reaction was stirred at room temperature overnight, concentrated in vacuo, and the residue was directly loaded on prep TLC and eluted with 100% EtOAc. The title compound 12 was isolated as a white solid (56 mg, 34%). (ES, m/z): 675.98 [M+H], ¹H-NMR (CD₃OD, 500 MHz, ppm): δ 7.98 (d, 1H, J=8 Hz), 7.46 (d, 1H, J=8 Hz), 7.40 (t, 2H, J=6 Hz), 7.26 (d, 2H, J=8 Hz), 7.23 (t, 1H, J=6 Hz), 6.39 (s, 1H), 5.13 (d, 1H, J=8 Hz), 4.96-5.04 (m, 1H), 4.49-4.56 (m, 1H), 4.34-4.42 (m, 2H), 3.72-4.00 (m, 1H), 2.68-2.76 (m, 2H), 1.39 (d, 3H, J=8 Hz), 1.16-1.29 (m, 21H).

The following compounds of the present invention were made using the methods described in the Example above and substituting the appropriate reactants and/or reagents.

| Compound | | Starting | |
|---|---|---|---|
| No. | Structure | Material | MS (M + H) |
| 13 | 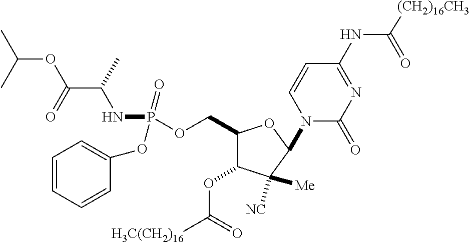 | Int-9b | 1068.93 |

### Example 12

### Preparation of Compound 14

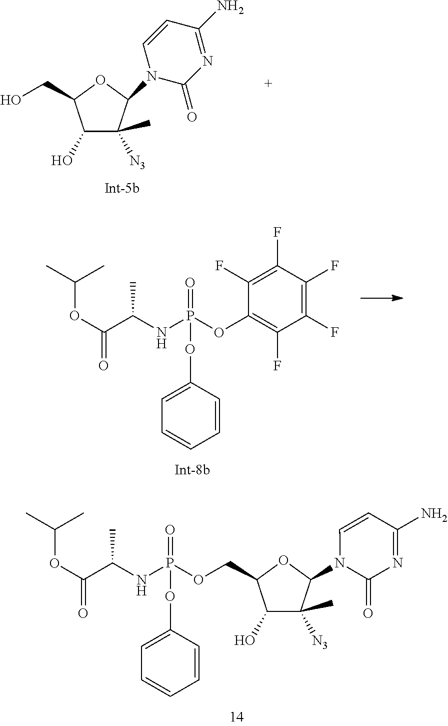 [SMILES: C[C@@]1(N=[N+]=[N-])[C@H](O)[C@@H](CO)O[C@H]1n1ccc(N)nc1=O ; CC(C)OC(=O)[C@H](C)NP(=O)(Oc1ccccc1)Oc1c(F)c(F)c(F)c(F)c1F ; CC(C)OC(=O)[C@H](C)NP(=O)(OC[C@H]1O[C@@H](n2ccc(N)nc2=O)[C@](C)(N=[N+]=[N-])[C@@H]1O)Oc1ccccc1]

To the starting nucleoside Int-5b (820 mg, 2.91 mmol) in a mixture of THF (50 mL) and NMP (16 mL) under nitrogen at 0° C. was added dropwise 2,2,6,6-Tetramethylpiperidinylmagnesium chloride lithium chloride complex solution (2.91 mL, 2.91 mmol) via syringe pump over 30 minutes. The reaction mixture was stirred for 2 hours at 0° C., then a solution of Int-8b (1317 mg, 2.91 mmol) in THF (6.0 mL) was added dropwise via syringe pump over 30 minutes. The reaction was allowed to slowly warm up to room temperature and stirred overnight. The reaction was quenched with saturated ammonium chloride (100 mL) and extracted with EtOAc (2×50 mL). The organic layer was washed with water, brine, dried over Na₂SO₄, filtered, and concentrated in vacuo. The residue was purified via a 24 g silica gel flash column chromatography (0 to 20% MeOH/CH₂Cl₂) to give the product 14 (1.06 g, 66%). (ES, m/z): 551.88 [M+H]⁺, ¹H-NMR (CD₃OD, 500 MHz, ppm): δ 7.69 (d, J=7.6 Hz, 1H), 7.37-7.40 (m, 2H), 7.26-7.28 (m, 2H), 7.20-7.23 (m, 1H), 5.85 (d, J=7.5 Hz, 1H), 4.93-4.97 (m, 1H), 4.49-4.58 (m, 2H), 4.34-4.38 (m, 1H), 4.07-4.09 (m, 1H), 3.89-3.97 (m, 2H), 1.34 (d, J=7.1 Hz, 3H), 1.30 (s, 3H), 1.22 (d, J=6.2 Hz, 6H).

### Example 13

### Preparation of Compound 15

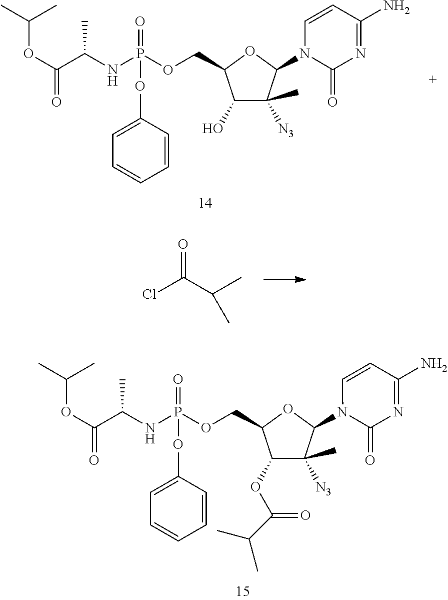

To a solution of 14 (170 mg, 0.308 mmol) in THF (5.0 mL) at 0° C. were added water (5.0 mL), triethylamine (0.129 mL, 0.925 mmol) and DMAP (4.14 mg, 0.034 mmol). The mixture was stirred at 0° C. for 5 minutes. And then isobutyryl chloride (0.081 mL, 0.771 mmol) was added to the reaction mixture slowly. The reaction was stirred for 90 minutes at 0° C., then quenched with aqueous 1N HCl to pH 6-7. The reaction mixture was with ethyl acetate (50 mL×2) and the combined ethyl acetate extracts were washed with water and brine, dried over Na₂SO₄, filtered, and concentrated in vacuo. The residue was purified via silica gel flash column chromatography (0 to 20% CH₃OH/CH₂Cl₂) to give the product 15 (134 mg, 70%). (ES, m/z): 621.95 [M+H]⁺, ¹H-NMR (CD₃OD, 500 MHz, ppm): δ 7.69 (d, J=7.6 Hz, 1H), 7.37-7.40 (m, 2H), 7.26-7.28 (m, 2H), 7.20-7.23 (m, 1H), 6.01 (s, 1H), 5.84 (d, J=7.6 Hz, 1H), 5.21-5.23 (m, 1H), 4.94-4.99 (m, 1H), 4.46-4.49 (m, 1H), 4.26-4.31 (m, 2H), 3.89-3.95 (m, 1H), 2.67-2.74 (m, 1H), 1.36 (d, J=7.1 Hz, 3H), 1.28 (s, 3H), 1.21-1.24 (m, 12H).

The following compounds of the present invention were made using the methods described in the Example above and substituting the appropriate reactants and/or reagents.

| Compound | | Starting | |
|---|---|---|---|
| No. | Structure | Material | MS (M + H) |
| 16 | 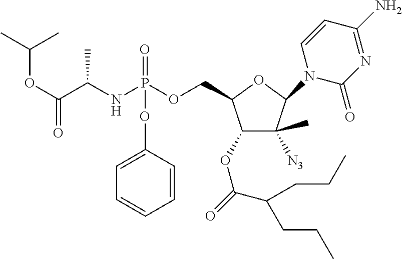 [SMILES: CCCC(CCC)C(=O)O[C@@H]1[C@@H](COP(=O)(N[C@@H](C)C(=O)OC(C)C)Oc2ccccc2)O[C@@H](n2ccc(N)nc2=O)[C@]1(C)N=[N+]=[N-]] | Compound 14 | 678.4 |
| 17 | 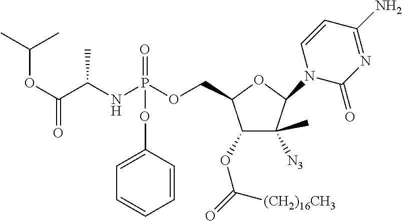 | Compound 14 | 818.4 |

### Example 14

### Preparation of Compound 18 and 19

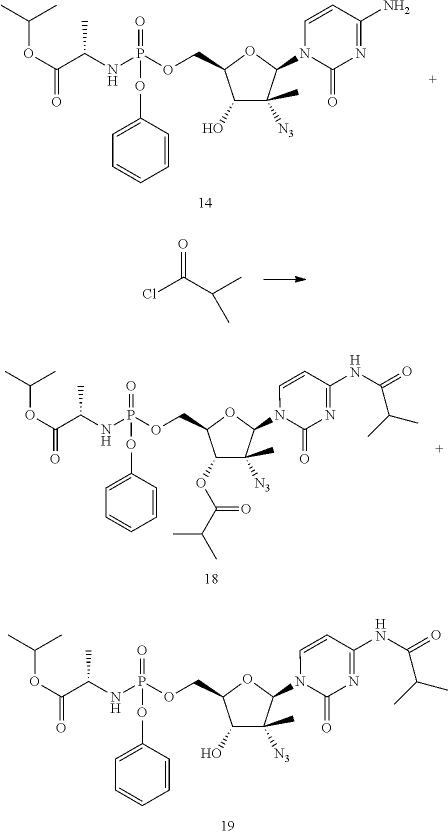

To a solution of 14 (227 mg, 0.412 mmol) in CH₂Cl₂ (4.0 mL) at room temperature were added DIPEA (0.359 mL, 2.058 mmol) and isobutyryl chloride (0.092 mL, 0.824 mmol). The reaction mixture was stirred at room temperature overnight. The reaction was quenched with water and extracted with CH₂Cl₂ (50 mL×2). The combined CH₂Cl₂ extracts were washed with brine, dried over Na₂SO₄, filtered, and concentrated in vacuo. The residue was purified by preparative TLC using 60% EtOAc/Hexane to give compounds 18 (54 mg, 19%) and 19 (151 mg, 59%).

Compound 18: (ES, m/z): 692.13 [M+H]⁺, ¹H-NMR (CD₃OD, 500 MHz, ppm): δ 8.04 (d, J=7.6 Hz, 1H), 7.45 (d, J=7.5 Hz, 1H), 7.37-7.40 (m, 2H), 7.27-7.29 (m, 2H), 7.19-7.22 (m, 1H), 6.06 (s, 1H), 5.25 (d, J=8.1 Hz, 1H), 4.93-5.02 (m, 1H), 4.47-4.50 (m, 1H), 4.29-4.35 (m, 2H), 3.91-3.97 (m, 1H), 2.65-2.76 (m, 2H), 1.38 (d, J=7.1 Hz, 3H), 1.27 (s, 3H), 1.18-1.25 (m, 18H).

Compound 19: (ES, m/z): 621.98 [M+H]⁺, ¹H-NMR (CD₃OD, 500 MHz, ppm): δ 8.05 (d, J=7.5 Hz, 1H), 7.46 (d, J=7.6 Hz, 1H), 7.37-7.40 (m, 2H), 7.27-7.29 (m, 2H), 7.19-7.22 (m, 1H), 5.98 (s, 1H), 4.93-4.99 (m, 1H), 4.50-4.54 (m, 1H), 4.38-4.42 (m, 1H), 4.12-4.14 (m, 1H), 3.99-4.01 (m, 1H), 3.90-3.96 (m, 1H), 2.64-2.70 (m, 1H), 1.36 (d, J=7.2 Hz, 3H), 1.30 (s, 3H), 1.18-1.22 (m, 12H).

The following compounds of the present invention were made using the methods described in the Example above and substituting the appropriate reactants and/or reagents.

| Compound | | Starting | |
|---|---|---|---|
| No. | Structure | Material | MS (M + H) |
| 20 | 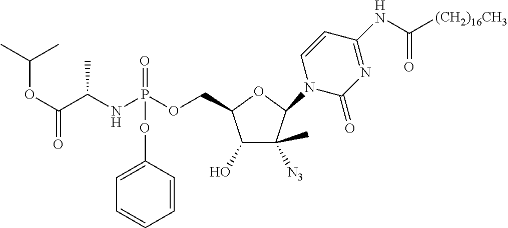 | Compound 14 | 818.3 |
| 21 | 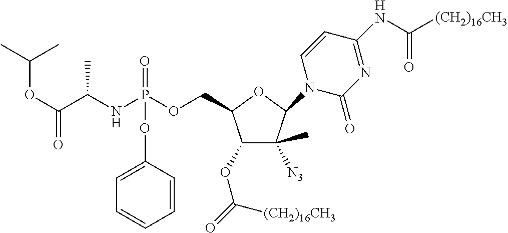 | Compound 14 | 1084.7 |

### Example 15

### Preparation of Compound 22

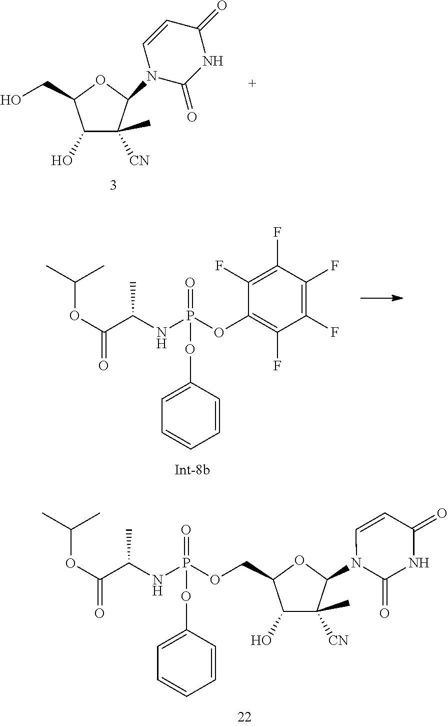 [SMILES: C[C@@]1(C#N)[C@H](O)[C@@H](CO)O[C@H]1n1ccc(=O)[nH]c1=O ; CC(C)OC(=O)[C@H](C)NP(=O)(Oc1ccccc1)Oc1c(F)c(F)c(F)c(F)c1F ; CC(C)OC(=O)[C@H](C)NP(=O)(OC[C@H]1O[C@@H](n2ccc(=O)[nH]c2=O)[C@](C)(C#N)[C@@H]1O)Oc1ccccc1]

To the starting nucleoside 3 (5.0 g, 18.71 mmol) in a mixture of THF (200 mL) and NMP (60 mL) under nitrogen at 0° C. was added dropwise of 2,2,6,6-Tetramethylpiperidinylmagnesium chloride lithium chloride complex solution (20.58 mL, 20.58 mmol) via syringe pump over 50 minutes. The reaction mixture was stirred for 85 minutes at 0° C., then a solution of Int-8b (9.75 g, 21.52 mmol) in THF (60.0 mL) was added dropwise via syringe pump over 60 minutes, allowed the reaction mixture slowly warm up to room temperature and stirred for 2 hours. The reaction was quenched with acetic acid (1.392 mL, 24.32 mmol), pH˜6-7, diluted with ethyl acetate, washed with water, brine, dried over Na₂SO₄, filtered, and concentrated in vacuo. The residue was purified via silica gel flash column chromatography (0 to 10% CH₃OH/CH₂Cl₂) to give the product 22 (6.27 g, 63%). (ES, m/z): 537.44 [M+H]⁺, ¹H-NMR (CD₃OD, 500 MHz, ppm): δ 7.66 (d, J=8.3 Hz, 1H), 7.37-7.40 (m, 2H), 7.26-7.28 (m, 2H), 7.20-7.23 (m, 1H), 6.34 (s, 1H), 5.65 (d, J=8.1 Hz, 1H), 4.95-5.00 (m, 1H), 4.50-4.54 (m, 1H), 4.36-4.40 (m, 1H), 4.11-4.13 (m, 1H), 4.02-4.03 (m, 1H), 3.89-3.95 (m, 1H), 1.36 (d, J=7.2 Hz, 3H), 1.30 (s, 3H), 1.23 (d, J=6.3 Hz, 6H).

### Example 16

### Preparation of Compound 23

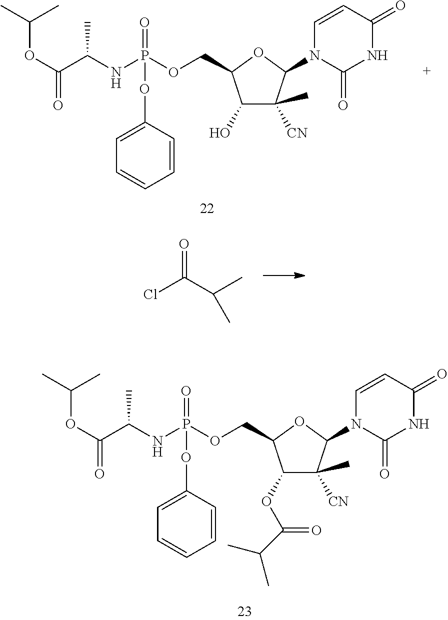

To a solution of 22 (4.5 g, 8.39 mmol) in THF (50 mL) at 0° C. were added water (50.0 mL), triethylamine (3.51 mL, 25.2 mmol) and DMAP (0.123 g, 1.007 mmol). The mixture was stirred at 0° C. for 5 minutes. And then isobutyryl chloride (1.758 mL, 16.78 mmol) was added to the reaction mixture slowly. The reaction was stirred for 90 minutes at 0° C., then quenched with aqueous 1N HCl to pH 6-7. Extract the mixture with ethyl acetate (200 mL×2). The combined ethyl acetate extracts were washed with water and brine, dried over Na₂SO₄, filtered, and concentrated in vacuo. The residue was purified via silica gel flash column chromatography (0 to 10% CH₃OH/CH₂Cl₂) to give the product 23 (4.8 g, 94%). (ES, m/z): 607.52 [M+H]⁺, ¹H-NMR (CD₃OD, 500 MHz, ppm): δ 7.61 (d, J=8.3 Hz, 1H), 7.37-7.40 (m, 2H), 7.26-7.27 (m, 2H), 7.20-7.23 (m, 1H), 6.32 (s, 1H), 5.65 (d, J=8.2 Hz, 1H), 5.13 (d, J=6.3 Hz, 1H), 4.96-5.01 (m, 1H), 4.46-4.50 (m, 1H), 4.33-4.37 (m, 2H), 3.90-3.96 (m, 1H), 2.68-2.73 (m, 1H), 1.37 (d, J=7.1 Hz, 3H), 1.29 (s, 3H), 1.21-1.27 (m, 12H).

The following compounds of the present invention were made using the methods described in the Example above and substituting the appropriate reactants and/or reagents.

| Compound | | Starting | |
|---|---|---|---|
| No. | Structure | Material | MS (M +H) |
| 24 | 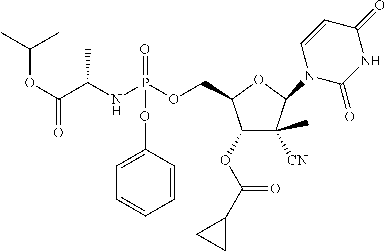 [SMILES: CC(C)OC(=O)[C@H](C)NP(=O)(OC[C@H]1O[C@@H](n2ccc(=O)[nH]c2=O)[C@](C)(C#N)[C@@H]1OC(=O)C1CC1)Oc1ccccc1] | Compound 22 | 605.4 |
| 25 | 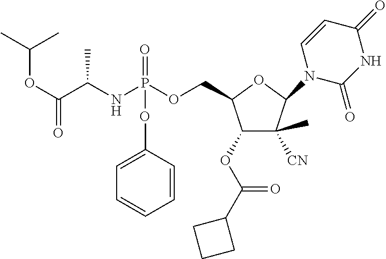 [SMILES: CC(C)OC(=O)[C@H](C)NP(=O)(OC[C@H]1O[C@@H](n2ccc(=O)[nH]c2=O)[C@](C)(C#N)[C@@H]1OC(=O)C1CCC1)Oc1ccccc1] | Compound 22 | 619.1 |
| 26 | 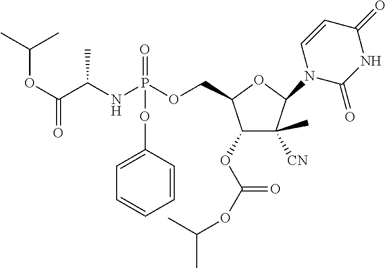 [SMILES: CC(C)OC(=O)O[C@@H]1[C@@H](COP(=O)(N[C@@H](C)C(=O)OC(C)C)Oc2ccccc2)O[C@@H](n2ccc(=O)[nH]c2=O)[C@]1(C)C#N] | Compound 22 | 623.1 |
| 27 | 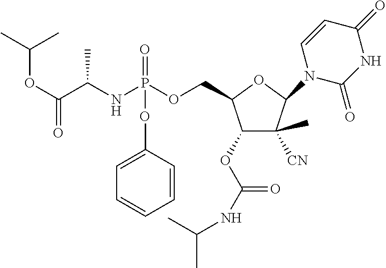 [SMILES: CC(C)NC(=O)O[C@@H]1[C@@H](COP(=O)(N[C@@H](C)C(=O)OC(C)C)Oc2ccccc2)O[C@@H](n2ccc(=O)[nH]c2=O)[C@]1(C)C#N] | Compound 22 | 622.3 |
| 28 | 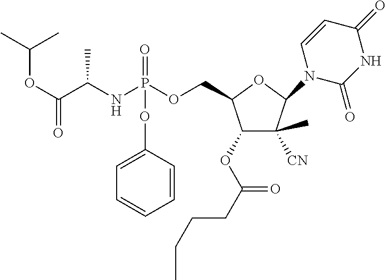 [SMILES: CCCCC(=O)O[C@@H]1[C@@H](COP(=O)(N[C@@H](C)C(=O)OC(C)C)Oc2ccccc2)O[C@@H](n2ccc(=O)[nH]c2=O)[C@]1(C)C#N] | Compound 22 | 621.2 |
| 29 | 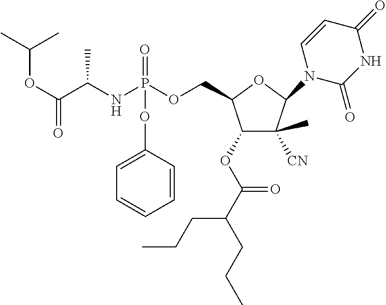 [SMILES: CCCC(CCC)C(=O)O[C@@H]1[C@@H](COP(=O)(N[C@@H](C)C(=O)OC(C)C)Oc2ccccc2)O[C@@H](n2ccc(=O)[nH]c2=O)[C@]1(C)C#N] | Compound 22 | 663.2 |
| 30 | 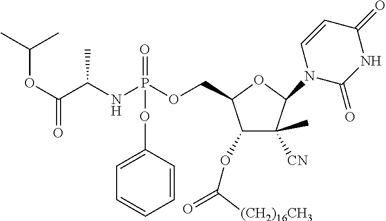 | Compound 22 | 803.4 |

### Example 17

### Preparation of Compound 31

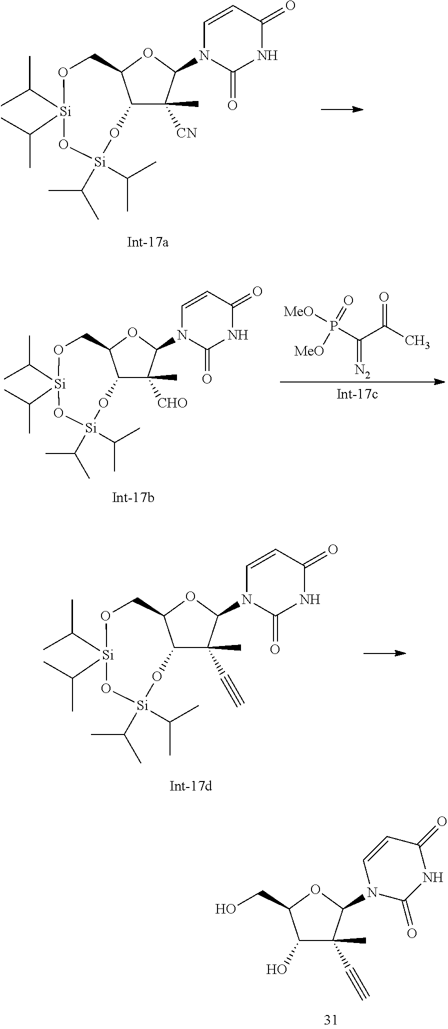

Int-17a was synthesized using procedures found in example 4.

### Step A—Synthesis of Compound Int-17b

Compound Int-17a (178 mg, 0.349 mmol) in dichloromethane (7 mL) was treated with bis(cyclopentadienyl)zirconium chloride hydride (900 mg, 3.49 mmol). The reaction was stirred at room temperature for 30 minutes. Saturated sodium potassium tartrate solution (10 mL) and ethyl acetate (10 mL) were added and the mixture was stirred until clear. The layers were separated and the aqueous phase was extracted with ethyl acetate (2×10 mL). The organic extracts were washed with brine (20 mL), dried over sodium sulfate, filtered and concentrated in vacuo to provide compound Int-17b (151 mg) as a yellow foam. [M+H]=513.3.

### Step B—Synthesis of Compound Int-17d

To a solution of Int-17b (186 mg, 0.362 mmol) in MeOH (3 mL) at 0° C. was added Int-17c (104 mg, 0.543 mmol) and K₂CO₃ (150 mg, 1.086 mmol). The reaction was stirred from 0° C. to room temperature for 6 hours. The reaction was diluted with water (10 mL) and extracted with ethyl acetate (2×20 mL). The combined organic phase was washed with brine (30 mL), dried over Na₂SO₄, filtered and concentrated in vacuo. The reaction was purified by preparative TLC (50% ethyl acetate/hexanes) to give Int-17d (42.5 mg). [M+H]=509.3.

### Step C—Synthesis of Compound 31

To a solution of compound Int-17d (48.5 mg, 0.095 mmol) in tetrahydrofuran (5 mL) was added tetrabutylammonium fluoride solution (0.19 mL, 0.191 mmol, 1 M in THF). The reaction was allowed to stir for 30 minutes. The reaction mixture was concentrated in vacuo and the residue obtained was purified by silica gel preparative TLC (5% methanol in dichloromethane) to provide 14.3 mg of compound 31. ¹H NMR (400 MHz, CD₃OD) δ 8.25 (d, J=8.0 Hz, 1H), 6.23 (s, 1H), 5.68 (d, J=8.0 Hz), 4.01-3.97 (m, 2H), 3.91 (d, J=9.2 Hz, 1H), 3.80 (dd, J=13.2, 2.8 Hz, 1H), 2.82 (s, 1H), 1.22 (s, 3H).

### Example 18

### Preparation of Compound 32

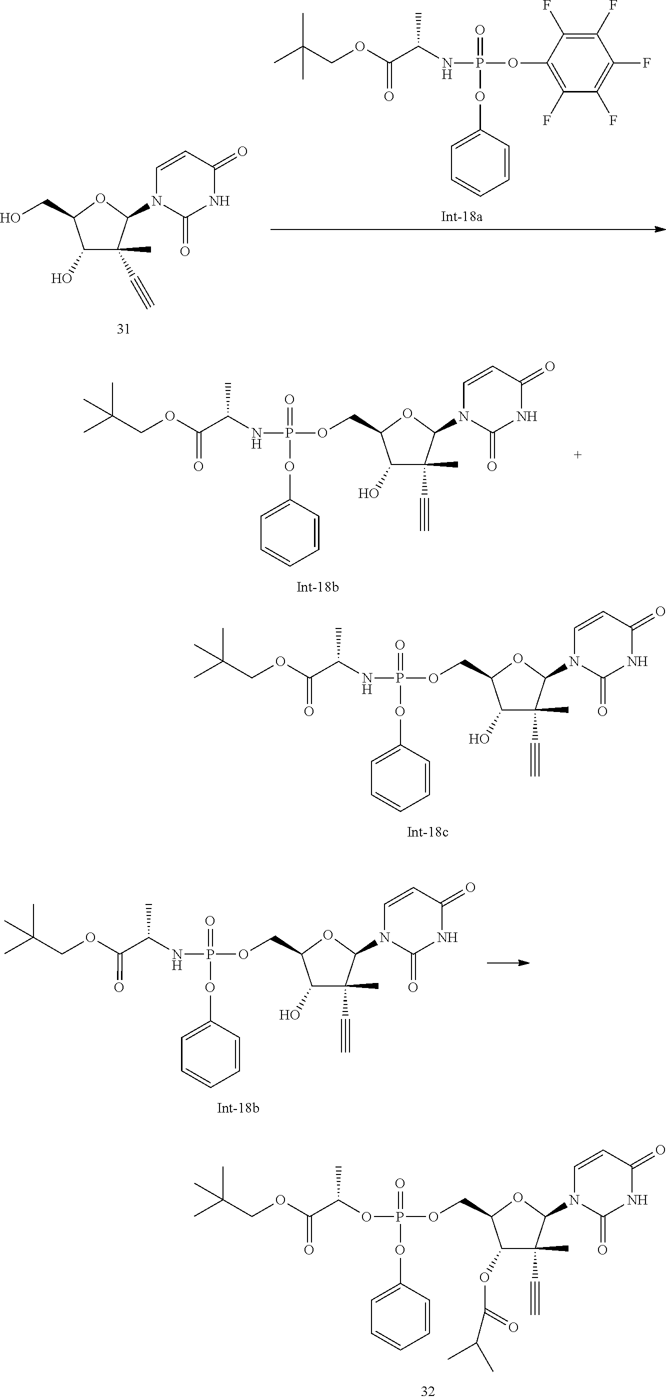

### Step A—Synthesis of Int-18b and Int-18c

To a solution of compound 1 (150 mg, 0.563 mmol) in THF (32 ml) at 0° C. was added tert-butylmagnesium chloride (1.408 ml, 1.408 mmol). After the reaction mixture was stirred for 15 minutes, then a solution of Int-18a (325 mg, 0.676 mmol, mixture of diastereomers synthesized using chemistry from example 8) in THF (9 ml) was added dropwise and the reaction was allowed to warm to room temperature. After stirring for 7 hours, the reaction mixture was quenched with saturated ammonium chloride and extracted with ethyl acetate. The organic extract was dried over sodium sulfate, filtered and concentrated in vacuo. The resulting residue was purified using flash column chromatography on silica gel (SNAP 50 g, 0-10% MeOH in DCM over 90 minutes) to provide 90 mg of diastereomers, which was separated by SFC to provide major isomer Int-18b (51.4 mg) and minor isomer Int-18c (5 mg)

Int-18b [M+H]=564.2 ¹H NMR (500 MHz, CD₃OD, ppm) 7.60 (d, 1H, J=8.1 Hz), 7.37 (dd, 2H, J=8.1, 7.7 Hz), 7.26 (d, 2H J=7.9 Hz), 7.20 (dd, 1H, J=7.5, 7.3 Hz), 6.24 (s, 1H), 5.60 (d, 1H, J=8.2 Hz), 4.46-4.54 (m, 1H), 4.34-4.42 (m, 1H), 4.10-4.19 (m, 1H), 3.96-4.04 (m, 1H), 3.84 (d, 2H, J=9.9 Hz), 3.74 (d, 1H, J=10.5 Hz), 2.80 (s, 1H), 1.39 (d, 3H, J=7.1 Hz), 1.20 (s, 3H), 0.92 (s, 9H)

Int-18c [M+H]=564.2 ¹H NMR (500 MHz, CD₃OD, ppm) 7.74 (d, 1H, J=8.1 Hz), 7.37 (dd, 2H, J=8.2, 7.6 Hz), 7.24 (d, 2H J=8.6 Hz), 7.19 (dd, 1H, J=7.5, 7.1 Hz), 6.25 (s, 1H), 5.62 (d, 1H, J=8.1 Hz), 4.54-4.60 (m, 1H), 4.39-4.45 (m, 1H), 4.13-4.20 (m, 1H), 3.92-4.04 (m, 1H), 3.80-3.87 (m, 2H), 3.78 (d, 1H, J=10.5 Hz), 2.85 (s, 1H), 1.35 (d, 3H, J=6.4 Hz), 1.18 (s, 3H), 0.93 (s, 9H)

### Step B—Synthesis of Compound 32

To a solution of compound Int-18b (37 mg, 0.066 mmol) in tetrahydrofuran (1.2 mL) and water (1.2 ml) was added triethylamine (0.0275 ml, 0.197 mmol) and DMAP (0.882 mg, 0.0072 mmol). After the reaction mixture was stirred at 0° C. for 5 min. isobutyl chloride (0.017 ml, 0.164 mmol) was added. The reaction mixture was allowed to warm at room temperature and stirred for 2 hours. LC-MS and TLC showed that starting material hasn't disappeared. To the reaction mixture was added triethylamine (0.11 ml, 0.788 mmol) and DMAP (3.53 mg, 0.028 mmol). and isobutyl chloride (0.068 ml, 0.656 mmol) and continued to stir at room temperature overnight. The reaction mixture was acidified with HCl (0.2 M) to adjust the PH 6-7 and extracted with EtOAc. The combined the organic extract was washed with water and brine, dried over sodium sulfate, filtered and concentrated in vacuo. The resulting residue was purified using flash column chromatography on silica gel (25 g SNAP, O-100% EtOAc in hexane 30 minutes, then hold at 100% EtOAc for 15 min.) to provide compound 32 (25 mg). [M+H]=634.3; ¹H NMR (500 MHz, CD₃OD, ppm) 7.66 (d, 1H, J=8.1 Hz), 7.37 (dd, 2H, J=8.4, 7.7 Hz), 7.26 (d, 2H J=8.1 Hz), 7.20 (dd, 1H, J=7.5, 7.2 Hz), 6.27 (s, 1H), 5.59 (d, 1H, J=8.2 Hz), 5.09 (d, 1H, J=7.5 Hz), 4.40-4.51 (m, 1H), 4.28-4.34 (m, 2H), 3.96-4.06 (m, 1H), 3.81 (d, 1H, J=10.4 Hz), 3.77 (d, 1H, J=10.5 Hz), 2.90 (s, 1H), 2.60-2.70 (m, 1H), 1.39 (d, 3H, J=7.1 Hz), 1.24 (d, 6H, J=7.1 Hz), 1.20 (s, 3H), 0.94 (s, 9H)

### Example 19

### Preparation of Compound 33

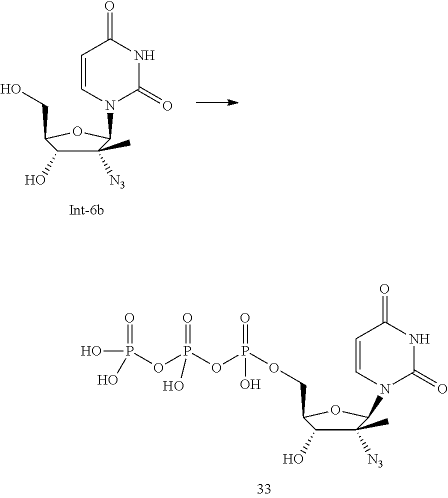 [SMILES: C[C@@]1(N=[N+]=[N-])[C@H](O)[C@@H](CO)O[C@H]1n1ccc(=O)[nH]c1=O ; C[C@@]1(N=[N+]=[N-])[C@H](O)[C@@H](COP(=O)(O)OP(=O)(O)OP(=O)(O)O)O[C@H]1n1ccc(=O)[nH]c1=O]

A solution of compound Int-6b (15 mg, 0.05 mmol, 1.00 equiv) in trimethyl phosphate (1.0 mL) was placed under nitrogen atmosphere and to the solution was added proton sponge (17 mg, 0.08 mmol, 1.50 equiv). The reaction was cooled to 0° C., then phosphoryl trichloride (32 mg, 0.21 mmol, 4.50 equiv) was added at 0° C. The resulting reaction was allowed to stir for 4 hours at 0° C., then a solution of pyrophosphate (200 mg, 0.37 mmol, 5.00 equiv), N,N-dimethylformamide (1.0 mL) and tributylamine (0.03 mL, 10.00 equiv) was added. The resulting reaction was allowed to stir for 1 hour at 0° C., then the reaction was then quenched by the addition of 3.0 mL of triethylammonium bicarbonate buffer (1M). The reaction mixture was concentrated in vacuo and the residue obtained was purified using Prep-HPLC with the following conditions (1#-Pre-HPLC-001(SHIMADZU)): Column, 1#-PrepC-008(Atlantis HILIC Silica 19*150 186003959 0110182551kk 03), mobile phase: acetonitrile and Water with 50 mmol ammonium bicarbonate (88% Water with 50 mmol ammonium bicarbonate down to 62% in 17 min); Detector, UV 220 & 254 nm. This provided 12 mg (43%) of compound 33 as a light yellow solid. (ES, m/z): 522 [M−H]⁻; ¹H-NMR (D₂O, 400 MHz, ppm): δ 7.86 (s, 1H), 6.05 (s, 1H), 5.85 (s, 1H), 3.99-4.70 (m, 4H), 1.40 (s, 3H); P-NMR (D₂O, 162 MHz, ppm): δ −6.09 (s, 1P), −11.12 (s, 1P), −21.33 (s, 1P).

The following compounds of the present invention were made using the methods described in the Example above and substituting the appropriate reactants and/or reagents.

| Compound | | Starting | MS ESI |
|---|---|---|---|
| No. | Structure | Material | deconvoluted |
| 34 | 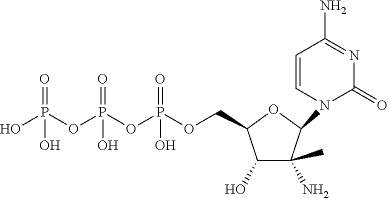 [SMILES: C[C@@]1(N)[C@H](O)[C@@H](COP(=O)(O)OP(=O)(O)OP(=O)(O)O)O[C@H]1n1ccc(N)nc1=O] | Compound 2 | 495.8 |
| 35 | 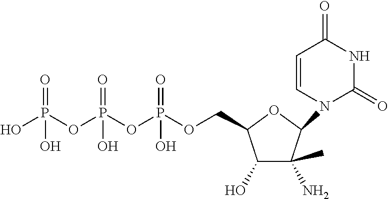 [SMILES: C[C@@]1(N)[C@H](O)[C@@H](COP(=O)(O)OP(=O)(O)OP(=O)(O)O)O[C@H]1n1ccc(=O)[nH]c1=O] | Compound 5 | 496.8 |
| 36 | 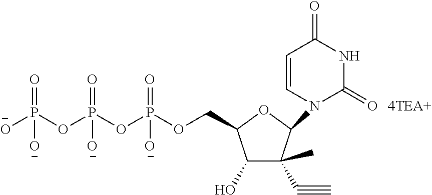 [SMILES: C#C[C@]1(C)[C@H](O)[C@@H](COP(=O)([O-])OP(=O)([O-])OP(=O)([O-])[O-])O[C@H]1n1ccc(=O)[nH]c1=O] | Compound 31 | 506.0 |

### Example 20

### Inhibition of HCV NS5B Polymerase by Nucleoside Triphosphate Analogs

To measure inhibition of the enzymatic activity of the HCV NS5B RNA-dependent RNA polymerase by the nucleoside triphosphate compounds of the present invention, a radiolabeled nucleotide incorporation assay was used. This assay is a modified version of the assay described in International Publication No. WO2002/057287. Briefly, 50 μL reactions containing 20 mM HEPES (pH 7.3); 7.5 mM DTT; 20 units/ml RNasIN; 1 μM each of ATP, GTP, UTP and CTP; 20 μCi/mL [³³P]-CTP; 10 mM MgCl; 60 mM NaCl; 100 μg/ml BSA; 0.021 DCoH heteropolymer RNA template; and 5 nM NS5B (1b-BKΔ55) enzyme are incubated at room temperature for 1 hour. The assay is then terminated by the addition of 500 mM EDTA (50 μl). The reaction mixture is transferred to a Millipore DE81 filter plate and the incorporation of labeled CTP is determined using Packard TopCount. Compound IC₅₀ values can then be calculated from experiments with 10 serial 3-fold dilutions of the inhibitor in duplicate. The intrinsic potency (Ki) of an NTP inhibitor is derived from its NS5B IC₅₀ using the Cheng-Prusoff equation for a competitive inhibitor, as described in Cheng et al., Biochem Pharmacol 22:3099-3108 (1973): Ki=IC₅₀/(1+[S]/Kₘ), where [S]=1 μM, and Kₘ is the concentration of cognate NTP yielding half-maximal enzyme activity in the assay absent exogenous inhibitors.

Data was obtained using this method for the NTP analogs of selected compounds below of the present invention, and is set forth below. This data indicates that the nucleoside triphosphate (NTP) of the compounds are potent and effective inhibitors of HCV NS5B polymerase. Data is also presented for comparative compounds, labeled as Compound B and Compound C.

| Compound | NTP Ki |
|---|---|
| No. | (μM) |
| 34 | 0.05 |
| 35 | 4.7 |
| 36 | 11 |

### Example 21

### Replicon Activity and Cytotoxicity Assays

To measure cell-based anti-HCV activity of the compounds of the present invention, replicon cells (1b-Con1) are seeded at 5000 cells/well in 96-well plates one day prior to treatment with a compound of the invention. Various concentrations of a test compound of the invention in DMSO are then added to the replicon cells, with the final concentration of DMSO at 0.5% and fetal bovine serum at 10% in the assay media. Cells are harvested three days post-dosing and the replicon RNA level is determined using real-time RT-PCR (Taqman assay) with GAPDH RNA as endogenous control. EC₅₀ values are calculated from experiments with 10 serial twofold dilutions of the inhibitor in triplicate. To measure cytotoxicity in replicon cells of an inhibitor, an MTS assay is performed according to the manufacturer's protocol for CellTiter 96 Aqueous One Solution Cell Proliferation Assay (Promega, Cat #G3580) three days post dosing on cells treated identically as in replicon activity assays. CC₅₀ is the concentration of inhibitor that yields 50% inhibition compared to vehicle-treated cells. Cytotoxicity in other types of cells can be measured using the same MTS protocol.

Data was obtained using this method for selected compounds of the present invention, and is set forth below. This data indicates that the compound possesses significant cytotoxicity windows over replicon activity.

| | Replicon (1b) | Cytotoxicity |
|---|---|---|
| Compound | EC₅₀ (μM) | (μM) |
| 6 | 0.125 | 50 |
| 7 | 0.425 | >100 |
| 8 | 5.09 | 6.54 |
| 10 | 9.5 | 10 |
| 11 | 1.8 | 25 |
| 12 | 10 | 76 |
| 13 | >100 | >100 |
| 15 | 0.6 | >100 |
| 16 | 0.1 | 13 |
| 17 | 8.2 | 9.9 |
| 18 | 15 | >100 |
| 19 | 8.5 | >100 |
| 20 | 3.6 | >100 |
| 23 | 0.7 | >100 |
| 24 | 6.3 | >100 |
| 25 | 0.7 | >100 |
| 26 | 1.0 | >100 |
| 27 | >100 | >100 |
| 28 | 0.7 | 76 |
| 29 | 0.19 | 20 |
| 32 | 10 | 59 |

### Example 22

### Mitochondrial Toxicity Assay

Mitochondrial toxicity in replicon cells of an inhibitor can be evaluated by its effect on the mitochondrial genome copy number relative to a nuclear gene control. Replicon cells are seeded at 60,000 cells/well in 6-well plates one day prior to inhibitor treatment. Various concentrations of an inhibitor in culture medium are added on the first day of treatment and dosing media are refreshed every three days thereafter. Cells are harvested at the indicated days post dosing; the total DNA is isolated using DNeasy Blood & Tissue Kit (Qiagen, Cat #69504) and quantitated by standard spectrophotometric methods. Two alternative sets of mitochondrial-specific DNA primer can be used: 1) 5′-CACCCAAGAACAGGGTTTGT-3′ (SEQ. ID. NO. 1) (F3212, forward), 5′-TGGCCATGGGTATGTTGTTAA-3′ (SEQ. ID. NO. 2) (R3319, reverse), 6-FAM-5′-TTACCGGGCTCTGCCATCT-3′-TAMRA (SEQ. ID. NO. 3) (probe) (see Bai et al., Ann NY Acad Sci 1011:304-309 (2004)); or 2) 5′-TGCCCGCCATCATCCTA-3′ (SEQ. ID. NO. 4) (COX II, forward), 5′-CGTCTGTTATGTAAAGGATGCGT-3′ (SEQ. ID. NO. 5) (COX II, reverse), 6-FAM-5′-TCCTCATCGCCCTCCCATCCC-3′-TAMRA (SEQ. ID. NO. 6) (probe) (see Stuyver et al., Antimicrob Agents Chemother 46:3854-3860 (2002)). Primers are used at 500 nM and probes at 200 nM in the Taqman quantitative PCR assay. The nuclear gene control quantitation is run in parallel for 18S DNA using ABI PDAR part #4310875 (20×). The ΔCT value (CT difference between mt DNA and 18S DNA) from inhibitor-treated cells is compared to that of vehicle-treated cells. Mitochondrial toxicity in other types of cells can be measured using the same protocol.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

What is claimed is:

1. A compound having the formula (I): 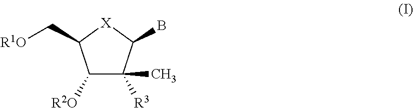
or a pharmaceutically acceptable salt thereof,
wherein:
B is: 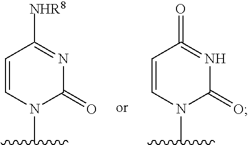
X is O, N, S or CH₂;
R¹ is H, 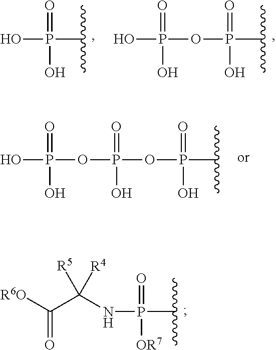
R² is H, —C(O)—(C₁-C₂₀ alkyl), —C(O)—(C₃-C₆ cycloalkyl), —C(O)O—(C₁-C₂₀ alkyl), —C(O)O—(C₃-C₆ cycloalkyl), —C(O)NH—(C₁-C₂₀ alkyl), —C(O)NH—(C₃-C₆ cycloalkyl) or 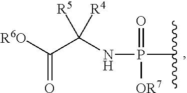
or R¹ and R² join to form a group having the formula: 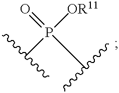
R³ is —CN, —N₃ or —C≡CH;
R⁴ is H, C₁-C₆ alkyl, C₃-C₇ cycloalkyl, phenyl or benzyl, wherein said C₁-C₆ alkyl can be optionally substituted with a group selected from halo, —OR¹², —SR¹², guanidino, —N(R¹²)₂, —C(O)OR¹², —C(O)N(R¹²)₂, —NHC(O)R¹², 5- or 6-membered monocyclic heteroaryl and 9- or 10-membered bicyclic heteroaryl, and wherein said phenyl group and said benzyl group can be optionally substituted with up to 2 groups, each independently selected from C₁-C₆ alkyl, halo and —OR¹²;
R⁵ is H, C₁-C₆ alkyl, C₃-C₇ cycloalkyl, phenyl or benzyl, wherein said C₁-C₆ alkyl can be optionally substituted with a group selected from halo, —OR¹², —SR¹², guanidino, —N(R¹²)₂, —C(O)OR¹², —C(O)N(R¹²)₂, —NHC(O)R¹², 5- or 6-membered monocyclic heteroaryl and 9- or 10-membered bicyclic heteroaryl, and wherein said phenyl group and said benzyl group can be optionally substituted with up to 2 groups, each independently selected from C₁-C₆ alkyl, halo and —OR¹²;
R⁶ is H, C₁-C₂₀ alkyl, C₂-C₂₀ alkenyl, —(C₁-C₃ alkylene)ₘ-(C₃-C₇ cycloalkyl), —(C₁-C₃ alkylene)ₘ-(C₆-C₁₀ aryl) or —(C₁-C₃ alkylene)ₘ-adamantyl, wherein said C₁-C₂₀ alkyl group, said C₂-C₂₀ alkenyl group, said C₆-C₁₀ aryl group and said adamantyl group can be optionally substituted with up to three groups, each independently selected from halo, —OR¹², —C(O)OR¹², CN, NO₂, C₁-C₆ haloalkyl, C₁-C₆ hydroxyalkyl, C₃-C₇ cycloalkyl, C₆-C₁₀ aryl, 5- or 6-membered monocyclic heteroaryl, 9- or 10-membered bicyclic heteroaryl, —N(R¹²)₂, —C(O)N(R¹²)₂—SR¹², —S(O)R¹², —S(O)₂R¹², —S(O)₂N(R¹²)₂, —NHC(O)R¹², —NHC(O)OR¹² and —NHC(O)N(R¹²)₂;
R⁷ is H, C₆-C₁₀ aryl, 5- or 6-membered monocyclic heteroaryl, 9- or 10-membered bicyclic heteroaryl, or —(C₁-C₃ alkylene)-C(O)O—(C₁-C₆ alkyl), wherein said C₆-C₁₀ aryl group, said 5- or 6-membered monocyclic heteroaryl group and said 9- or 10-membered bicyclic heteroaryl group can be optionally substituted with R¹³;
R⁸ is H or —C(O)—(C₁-C₂₀ alkyl);
R¹¹ is H, C₁-C₂₀ alkyl, C₂-C₂₀ alkenyl, —(C₁-C₃ alkylene)ₘ-(C₃-C₇ cycloalkyl), C₃-C₇ cycloalkyl, —(C₁-C₃ alkylene)ₘ-C₆-C₁₀ aryl or —(C₁-C₃ alkylene)ₘ-adamantyl, 5- or 6-membered monocyclic heteroaryl, 9- or 10-membered bicyclic heteroaryl, wherein said C₁-C₂₀ alkyl group, said C₂-C₂₀ alkenyl group, said C₆-C₁₀ aryl group, said adamantyl group, said 5- or 6-membered monocyclic heteroaryl group and said 9- or 10-membered bicyclic heteroaryl group can be optionally substituted with up to five groups, each independently selected from C₁-C₆ alkyl, C₂-C₆ alkenyl, C₂-C₆ alkynyl, C₃-C₇ cycloalkyl, C₆-C₁₀ aryl, 5- or 6-membered monocyclic heteroaryl, 9- or 10-membered bicyclic heteroaryl, halo, —OR¹², —SR¹², —S(O)R¹², —S(O)₂R¹², —S(O)₂N(R¹²)₂, C₁-C₆ haloalkyl, C₁-C₆ hydroxyalkyl, —O—(C₁-C₆ haloalkyl), —CN, —NO₂, —N(R¹²)₂, —C(O)OR¹², —C(O)N(R¹²)₂ and —NHC(O)R¹², —NHC(O)OR¹² and —NHC(O)N(R¹²)₂;
each occurrence of R¹² is independently H, C₁-C₁₀ alkyl, C₁-C₆ haloalkyl, C₁-C₆ hydroxyalkyl, —(C₁-C₃ alkylene)ₘ-(C₃-C₇ cycloalkyl), —(C₁-C₃ alkylene)ₘ-(C₆-C₁₀ aryl), —(C₁-C₃ alkylene)ₘ-(4 to 7-membered heterocycloalkyl), —(C₁-C₃ alkylene)ₘ-(5- or 6-membered monocyclic heteroaryl) or —(C₁-C₃ alkylene)ₘ-(9- or 10-membered bicyclic heteroaryl), wherein said C₃-C₇ cycloalkyl group, said C₆-C₁₀ aryl group, said 4 to 7-membered heterocycloalkyl group, said-5- or 6-membered monocyclic heteroaryl group or said 9- or 10-membered bicyclic heteroaryl group can be optionally substituted with R¹⁶;
R¹³ represents from one to five substituent groups, each independently selected from C₁-C₆ alkyl, halo, —OR¹², —SR¹², C₁-C₆ haloalkyl, C₁-C₆ hydroxyalkyl, —O—(C₁-C₆ haloalkyl), —CN, —NO₂, —N(R¹²)₂, —C(O)OR¹², —C(O)N(R¹²)₂ and —NHC(O)R¹², or any two R¹³ groups on adjacent ring carbon atoms can combine to form —O—R¹⁴—O—;
R¹⁴ is —[C(R¹⁵)₂]ₙ—;
each occurrence of R¹⁵ is independently H or C₁-C₆ alkyl;
R¹⁶ represents from one to five substituent groups, each independently selected from C₁-C₆ alkyl, C₂-C₆ alkenyl, C₂-C₆ alkynyl, halo, —OR¹⁷, —SR¹⁷, C₁-C₆ haloalkyl, C₁-C₆ hydroxyalkyl, —O—(C₁-C₆ haloalkyl), —CN, —NO₂, —N(R¹⁷)₂, —C(O)OR¹⁷, —C(O)N(R¹⁷)₂ and —NHC(O)R¹⁷;
each occurrence of R¹⁷ is independently H, C₁-C₆ alkyl, C₁-C₆ haloalkyl, C₁-C₆ hydroxyalkyl, —(C₁-C₃ alkylene)ₘ-(C₃-C₇ cycloalkyl), —(C₁-C₃ alkylene)ₘ-(C₆-C₁₀ aryl), —(C₁-C₃ alkylene)ₘ-(4 to 7-membered heterocycloalkyl), —(C₁-C₃ alkylene)ₘ-(5- or 6-membered monocyclic heteroaryl) or —(C₁-C₃ alkylene)ₘ-(9- or 10-membered bicyclic heteroaryl);
each occurrence of m is independently 0 or 1, and
each occurrence of n is independently 1, 2, or 3.

2. The compound of claim 1, wherein X is O.

3. The compound of claim 2, wherein B is:

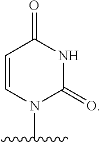 [SMILES: *n1ccc(=O)[nH]c1=O]

4. The compound of claim 2, wherein B is:

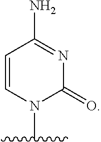 [SMILES: *n1ccc(N)nc1=O]

5. The compound of claim 1, wherein R¹ is:

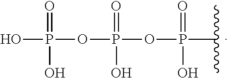 [SMILES: *P(=O)(O)OP(=O)(O)OP(=O)(O)O]

6. The compound of claim 1, wherein R¹ and R² join to form a group having the formula:

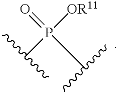

7. The compound of claim 1 having the formula: 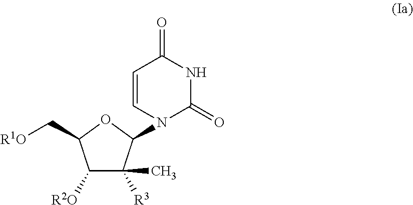
or a pharmaceutically acceptable salt thereof,
wherein:
R¹ is: 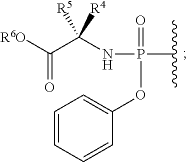
R² is H or —C(O)(C₁-C₆ alkyl);
R³ is —CN, —N₃ or —C≡CH;
R⁴ is H;
R⁵ is C₁-C₆ alkyl; and
R⁶ is C₁-C₆ alkyl.

8. The compound of claim 7, wherein R³ is —CN.

9. The compound of claim 7, wherein R³ is —N₃.

10. The compound of claim 7, wherein R³ is —C≡CH.

11. The compound of claim 7, wherein R¹ is: 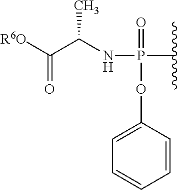
and R⁶ is C₁-C₆ alkyl.

12. The compound of claim 11, wherein R¹ is:

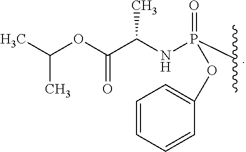 [SMILES: *P(=O)(N[C@@H](C)C(=O)OC(C)C)Oc1ccccc1]

13. The compound of claim 7, wherein R² is H.

14. The compound claim 7, wherein R² is —C(O)(C₁-C₆ alkyl).

15. The compound of claim 14, wherein R² is —C(O)CH(CH₃)₂.

16. The compound of claim 1 having the structure: 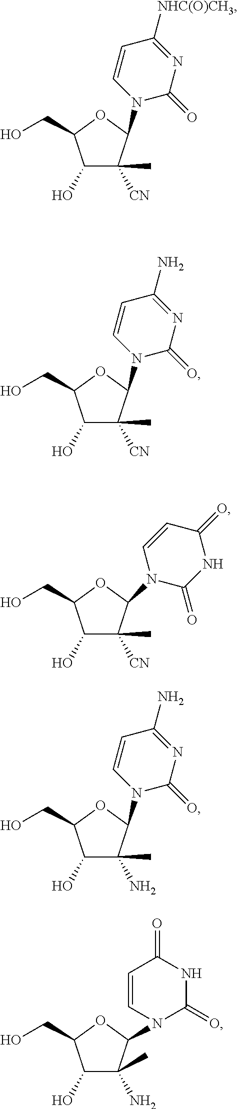 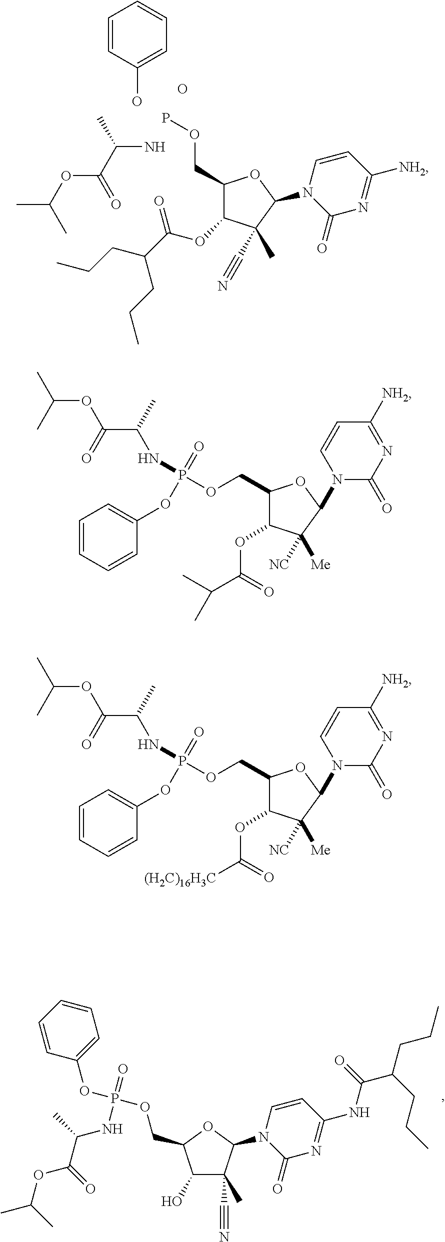 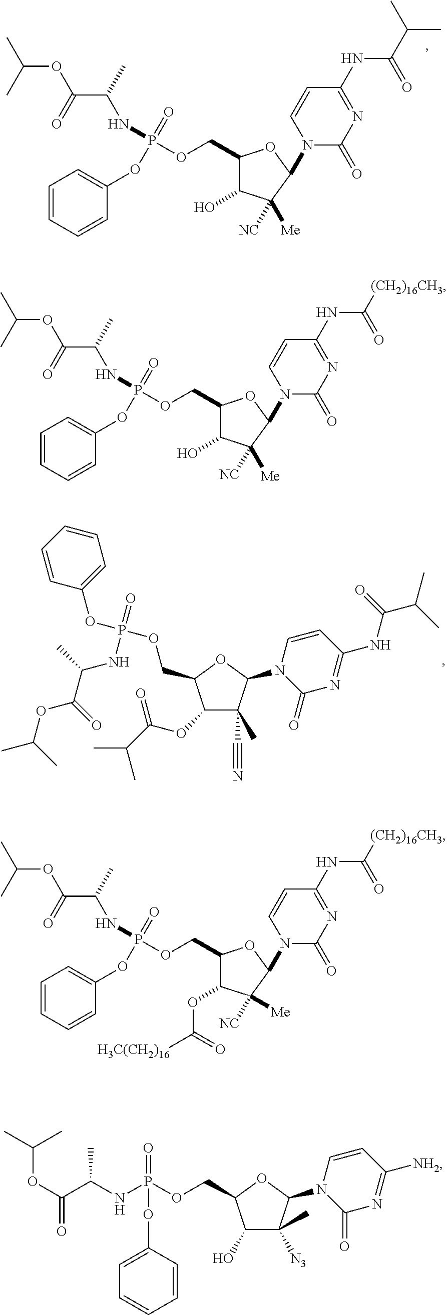 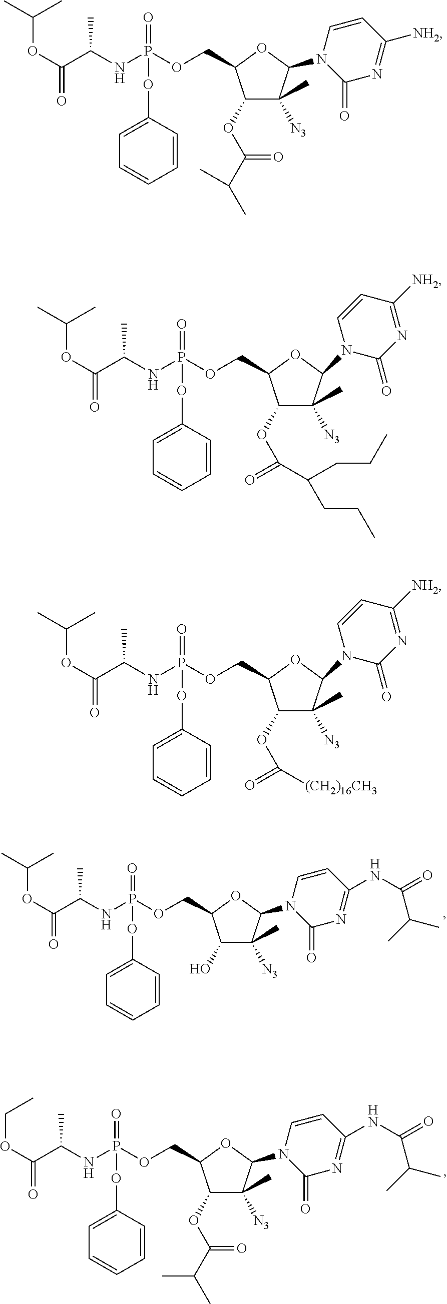 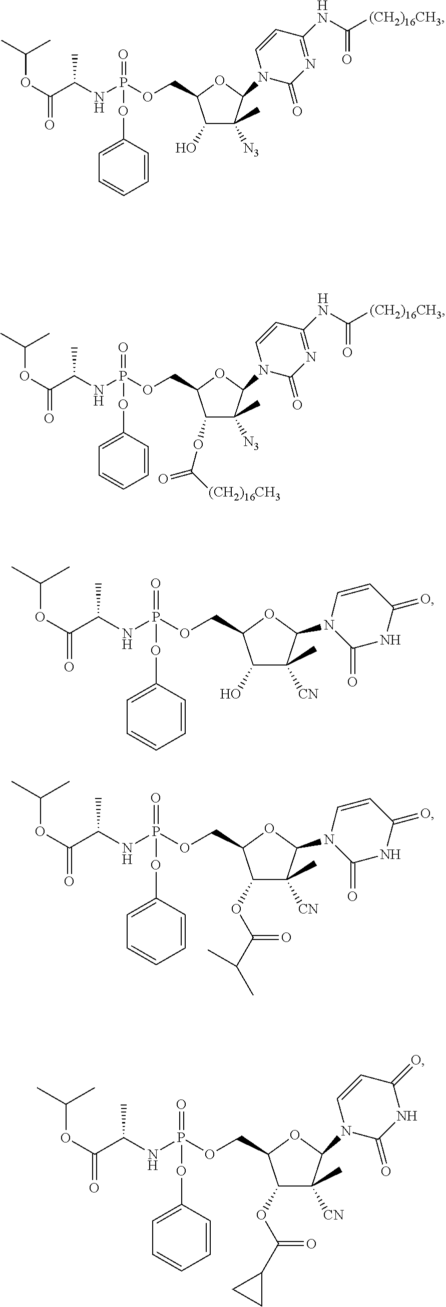 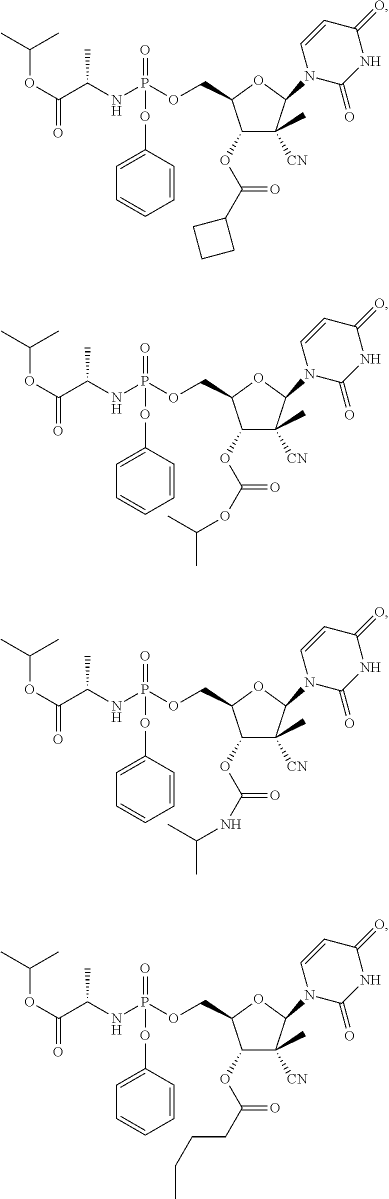 [SMILES: CC(C)OC(=O)[C@H](C)NP(=O)(OC[C@H]1O[C@@H](n2ccc(=O)[nH]c2=O)[C@](C)(C#N)[C@@H]1OC(=O)C1CCC1)Oc1ccccc1 ; CC(C)OC(=O)O[C@@H]1[C@@H](COP(=O)(N[C@@H](C)C(=O)OC(C)C)Oc2ccccc2)O[C@@H](n2ccc(=O)[nH]c2=O)[C@]1(C)C#N ; CC(C)NC(=O)O[C@@H]1[C@@H](COP(=O)(N[C@@H](C)C(=O)OC(C)C)Oc2ccccc2)O[C@@H](n2ccc(=O)[nH]c2=O)[C@]1(C)C#N ; CCCCC(=O)O[C@@H]1[C@@H](COP(=O)(N[C@@H](C)C(=O)OC(C)C)Oc2ccccc2)O[C@@H](n2ccc(=O)[nH]c2=O)[C@]1(C)C#N] 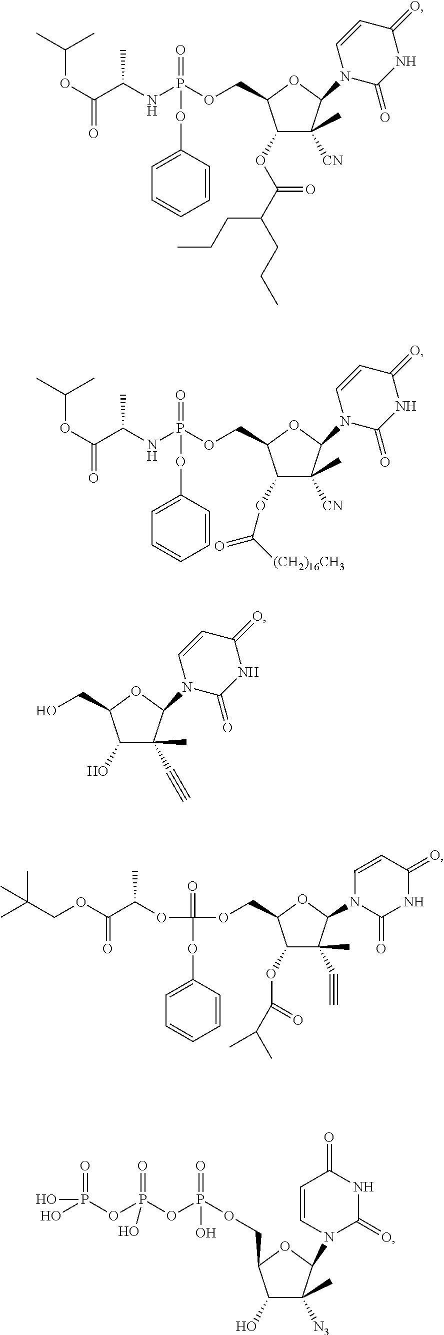 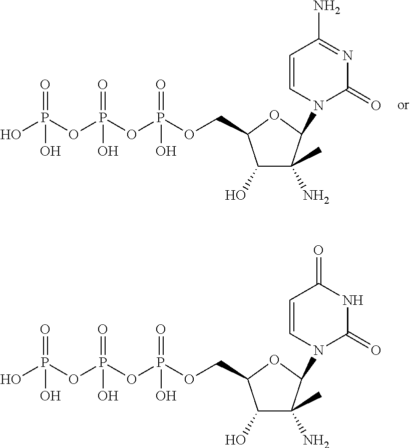 [SMILES: C[C@@]1(N)[C@H](O)[C@@H](COP(=O)(O)OP(=O)(O)OP(=O)(O)O)O[C@H]1n1ccc(N)nc1=O ; C[C@@]1(N)[C@H](O)[C@@H](COP(=O)(O)OP(=O)(O)OP(=O)(O)O)O[C@H]1n1ccc(=O)[nH]c1=O]
or a pharmaceutically acceptable salt thereof.

17. A pharmaceutical composition comprising an effective amount of the compound of claim 1 or a pharmaceutically acceptable salt thereof, and a pharmaceutically acceptable carrier.

18. The pharmaceutical composition according to claim 17, further comprising a second therapeutic agent selected from the group consisting of HCV antiviral agents, immunomodulators, and anti-infective agents.

19. The pharmaceutical composition according to claim 18, further comprising a third therapeutic agent selected from the group consisting of HCV protease inhibitors, HCV NS5A inhibitors and HCV NS5B polymerase inhibitors.

20. The use of the compound of claim 1, or a pharmaceutically acceptable salt thereof, in the preparation of a medicament for inhibiting HCV NS5B activity or for preventing and/or treating infection by HCV in a patient in need thereof.

21. A method of treating a patient infected with HCV comprising administering to said patient an amount of the compound of claim 1, or a pharmaceutically acceptable salt thereof, effective to prevent and/or treat infection by HCV in said patient.

22. The method according to claim 21, further comprising the step of administering to said patient a second therapeutic agent selected from the group consisting of HCV antiviral agents, immunomodulators, and anti-infective agents.

23. The method according to claim 22, further comprising the step of administering to said patient a third therapeutic agent selected from the group consisting of HCV protease inhibitors, HCV NS5A inhibitors and HCV NS5B polymerase inhibitors.
